(19) United States
(12) Patent Application Publication
(10) Pub. No.: US 2025/0243492 A1
(43) Pub. Date: Jul. 31, 2025

(54) COMPOSITIONS AND METHODS FOR TREATMENT OF PRION DISEASES

(71) Applicant: Atalanta Therapeutics, Inc., Boston, MA (US)

(72) Inventors: Matthew HASSLER, Boston, MA (US); Daniel CURTIS, Belmont, MA (US)

(21) Appl. No.: 18/698,544

(22) PCT Filed: Oct. 4, 2022
(86) PCT No.: PCT/US2022/077552

§ 371 Date: Apr. 4, 2024

Related U.S. Application Data
(60) Provisional application No. 63/252,035, filed on Oct. 4, 2021.

(51) Int. Cl. C12N 15/113

(52) U.S. Cl. CPC C12N 15/1138; C12N 2310/14; C12N 2310/31; C12N 2310/322

(57) ABSTRACT

The present disclosure provides single- or double-stranded interfering RNA molecules (e.g., siRNA) that target a PRNP gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a prion disease or a high-penetrance PRNP mutation.

### SEQUENCE LISTING

The instant application contains a Sequence Listing which has been submitted electronically in XML format and is hereby incorporated by reference in its entirety. Said XML copy, created on Sep. 1, 2022, is named 51436-032WO2_Sequence_Listing_9_1_22_ST26.xml and is 647,440 bytes in size.

### BACKGROUND

Prion diseases are a group of fatal, neurodegenerative disease affecting humans and other mammals. These diseases often present as rapidly progressive dementia, with some instances presenting as ataxia, insomnia, and slowly progressive dementia. The diseases are traceable to the cellular prion protein (PrP^{C}), which is predominantly expressed in the brain and lymphatic tissues and is encoded by the PRNP gene. Misfolding of PrP^{C} can occur due to environmental or genetic triggers, and the misfolded isoform (PrP^{SC}) induces the further misfolding of PrP^{C}. Thus, the production of the disease-causing PrP^{SC} is self-propagating.

PrP^{SC} is implicated in the disease regardless of the clinical subtype (Creutzfeldt-Jakob disease, fatal familial insomnia, or Gerstmann-Straussler-Scheinker Syndrome) or etiology (sporadic, genetic, or acquired). Thus, there is a need for methods of reducing prion protein expression as a treatment and preventative measure for prion diseases.

### SUMMARY OF THE INVENTION

The misfolding of prion proteins, and by extension, the triggering of a prion disease, can be initiated by environmental or genetic triggers. The gene that encodes prion protein, PRNP, is associated with various high-penetrance mutations that lead to prion disease. High-penetrance mutations of PRNP include, for example, E200K, D178N, P102L, 6-OPRI, 5-OPRI, A117V, and P105L with respect to the amino acid sequence of human prion protein (PrP; UNIPROT™ Accession No. P04156-1).

The present disclosure provides compositions and methods for reduction of prion protein (PrP) expression in the brain by way of small interfering RNA (siRNA)-mediated silencing of PRNP transcripts.

The compositions and methods provide the benefit of exhibiting high selectivity toward PRNP over other central nervous system (CNS) genes.

The siRNA molecules of the disclosure can be used to silence the PRNP gene, thereby preventing the translation of the gene and reducing prion protein expression in the brain. This reduction of prion protein levels thus prevents disease onset or progression in the brain. The siRNA molecules of the disclosure can be administered to pre-symptomatic individuals identified as carrying a high-penetrance PRNP mutation. Reduction of prion protein expression is expected to be well-tolerated, as there are known to be healthy adults with heterozygous loss of function prion mutations. The siRNA molecules of the disclosure can be delivered directly to the CNS of a subject in need of PRNP silencing by way of, for example, injection intrathecally, intracerebroventricularly, intrastriatally, or by intra-cisterna magna injection by catheterization.

In an aspect, the disclosure provides a small interfering RNA (siRNA) molecule containing an antisense strand and sense strand having complementarity to the antisense strand. The antisense strand has complementarity sufficient to hybridize to a region within a prion protein (PRNP) mRNA transcript having the nucleic acid sequence of any one of SEQ ID NOs: 493-738. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 15 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 16 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 17 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 18 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 19 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 20 contiguous nucleobases within the PRNP mRNA transcript having the nucleic acid sequence of any one of SEQ ID NOs: 493-738. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 21 contiguous nucleobases within the PRNP mRNA transcript having the nucleic acid sequence of any one of SEQ ID NOs: 493-738.

In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the PRNP mRNA transcript having the nucleic acid sequence of any one of SEQ ID NOs: 493-738.

In some embodiments, the antisense strand has at least 75% complementarity to the region within the PRNP mRNA transcript having the nucleic acid sequence of any one of SEQ ID NOs: 493-738.

For example, the antisense strand may have 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the PRNP mRNA transcript having the nucleic acid sequence of any one of SEQ ID Nos: 493-738 In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the PRNP RNA transcript having the nucleic acid sequence of any one of SEQ ID NOs: 493-738.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID NOs: 493-738.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID NOs: 493-738.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID NOs: 493-738.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID NOs: 493-738.

In some embodiments, the antisense strand has from 21 to 30 contiguous nucleotides (e.g., 21, 22, 23, 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID Nos: 493-738.

In some embodiments, the antisense strand has from 24 to 30 contiguous nucleotides (e.g., 24, 25, 26, 27, 28, 29, or 30 contiguous nucleotides) that are fully complementary to a contiguous polynucleotide segment of equal length within the region of the PRNP RNA transcript having the nucleic acid sequence of any one of SEQ ID NOs: 493-738.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the PRNP mRNA transcript having the nucleic acid sequence of any one of SEQ ID Nos: 493-738.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the PRNP mRNA transcript having the nucleic acid sequence of any one of SEQ ID NOs: 493-738.

In some embodiments, the antisense strand has 21 contiguous nucleotides that are fully complementary to a contiguous polynucleotide segment of equal length within the region of the PRNP mRNA transcript having the nucleic acid sequence of any one of SEQ ID NOs: 493-738.

In some embodiments, the antisense strand has 30 contiguous nucleotides that are fully complementary to a contiguous polynucleotide segment of equal length within the region of the PRNP RNA transcript having the nucleic acid sequence of any one of SEQ ID NOs: 493-738.

In some embodiments, the antisense strand has 9 or fewer nucleotide mismatches relative to the region of the PRNP RNA transcript having the nucleic acid sequence of any one of SEQ ID NOs: 493-738, optionally wherein the antisense strand comprises 8 or fewer, 7 or fewer, 6 or fewer, 5 or fewer, 4 or fewer, 3 or fewer, 2 or fewer, or only 1 mismatch relative to the region of the PRNP RNA transcript having the nucleic acid sequence of any one of SEQ ID NOs: 493-738.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-246.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 1-246.

In some embodiments, the antisense strand has a nucleic acid sequence that is at least 95% identical (e.g., 95%, 96%, 97%, 98%, 99%, or 100% identical) to the nucleic acid sequence of SEQ ID NOs: 1-246, optionally wherein the antisense strand has a nucleic acid sequence that is at least 96%, 97%, 98%, or 99% identical to the nucleic acid sequence of any one of SEQ ID NOs: 1-246.

In some embodiments, the antisense strand has the nucleic acid sequence of any one of SEQ ID NOs: 1-246.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 247-492.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 247-492.

In some embodiments, the sense strand has a nucleic acid sequence that is at least 95% identical (e.g., 95%, 96%, 97%, 98%, 99%, or 100% identical) to the nucleic acid sequence of SEQ ID NOs: 247-492, optionally wherein the sense strand has a nucleic acid sequence that is at least 96%, 97%, 98%, or 99% identical to the nucleic acid sequence of any one of SEQ ID NOs: 247-492.

In some embodiments, the siRNA molecule has a sense strand having the nucleic acid sequence of any one of SEQ ID NOs: 247-492.

In some embodiments, the antisense strand has a structure represented by Formula I, wherein Formula I is, in the 5′-to-3′ direction:

A-B-(A′)ⱼ-C-P²-D-P¹-(C′-P¹)ₖ-C′ Formula I;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

A-B-(A′)ⱼ-C-P²-D-P¹-(C-P¹)ₖ-C′ Formula II;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

A-(A′)ⱼ-C-P²-B-(C-P¹)ₖ-C′ Formula IV;
wherein A is represented by the formula C-P¹-D-P¹;
each A′, independently, is represented by the formula C-P²-D-P²;
B is represented by the formula D-P¹-C-P¹-D-P¹;
each C, independently, is a 2′-O-Me ribonucleoside;
each C′, independently, is a 2′-O-Me ribonucleoside or a 2′-F ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

E-(A′)ₘ-C-P²-F Formula V;

wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

A-Bⱼ-E-Bₖ-E-F-Gi-D-P¹-C′ Formula VI;
wherein A is represented by the formula C-P¹-D-P¹;
each B, independently, is represented by the formula C-P²;
each C, independently, is a 2′-O-Me ribonucleoside;
each C′, independently, is a 2′-O-Me ribonucleoside or a 2′-F ribonucleoside;
each D, independently, is a 2′-F ribonucleoside;
each E, independently, is represented by the formula D-P²-C-P²;
F is represented by the formula D-P¹-C-P¹;
each G, independently, is represented by the formula C-P¹;
each P¹ is, independently, a phosphorothioate internucleoside linkage;
each P² is, independently, a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

H-Bₘ-Iₙ-A′-Bₒ-H-C Formula VII;
wherein A′ is represented by the formula C-P²-D-P²;
each H, independently, is represented by the formula (C-P¹)₂;
each I, independently, is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

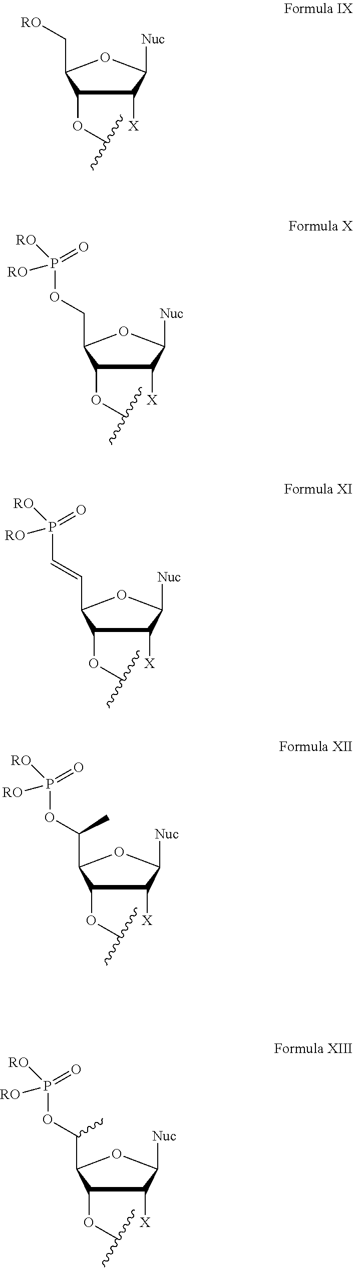

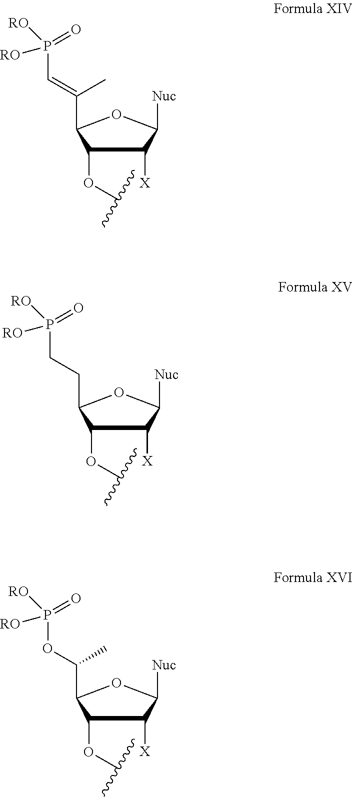

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

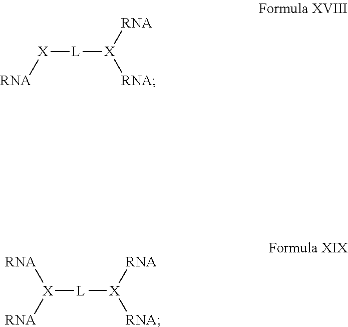

RNA-L-RNA Formula XVII;

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

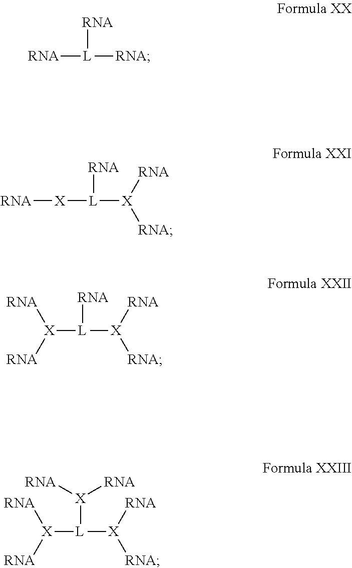

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

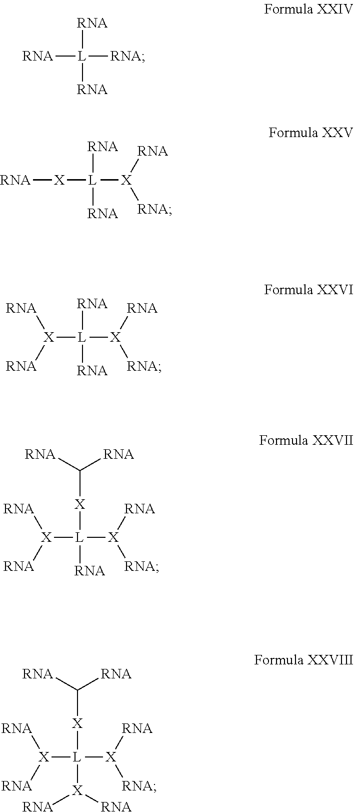

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

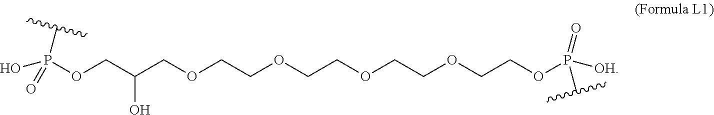 [SMILES: *P(=O)(O)OCCOCCOCCOCCOCC(O)COP(*)(=O)O]

In some embodiments, the linker includes a structure of Formula L2:

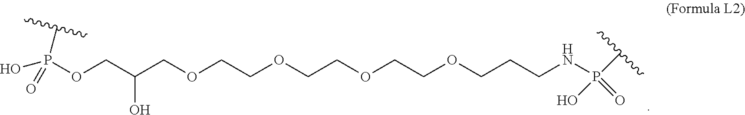 [SMILES: *P(=O)(O)NCCCOCCOCCOCCOCC(O)COP(*)(=O)O]

In some embodiments, the linker includes a structure of Formula L3:

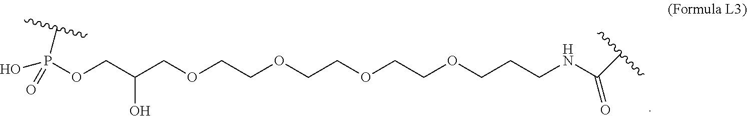 [SMILES: *C(=O)NCCCOCCOCCOCCOCC(O)COP(*)(=O)O]

In some embodiments, the linker includes a structure of Formula L4:

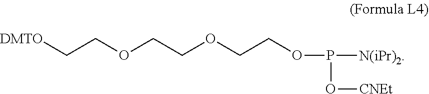

In some embodiments, the linker includes a structure of Formula L5:

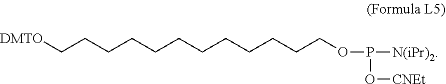

In some embodiments, the linker includes a structure of Formula L6:

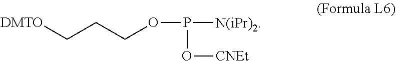

In some embodiments, the linker includes a structure of Formula L7:

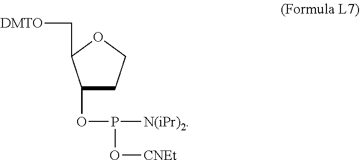

In some embodiments, the linker includes a structure of Formula L8:

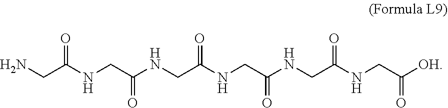 [SMILES: NCC(=O)NCC(=O)NCC(=O)NCC(=O)NCC(=O)NCC(=O)O]

In some embodiments, the linker includes a structure of Formula L9:

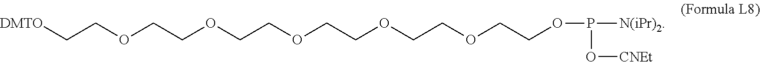

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages.

In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides.

In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides.

In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to the central nervous system (CNS) of a subject diagnosed as having a prion disease, or a subject identified as having a high-penetrance PRNP mutation, by administering an siRNA molecule or a pharmaceutical composition of any of the preceding aspects or embodiments of the disclosure to the subject.

In a further aspect, the disclosure provides a method of treating a prion disease in a subject in need thereof by administering a therapeutically effective amount of an siRNA molecule or a pharmaceutical composition of any of the preceding aspects or embodiments of the disclosure to the CNS of the subject.

In some embodiments, the prion disease is Creutzfeldt-Jakob Disease. In some embodiments, the prion disease is Gerstmann-Straussler-Scheinker Syndrome. In some embodiments, the prion disease is Fatal Familial Insomnia. In some embodiments, the prion disease is kuru. In some embodiments, the prion disease is bovine spongiform encephalopathy. In some embodiments, the prion disease is scrapie. In some embodiments, the prion disease is chronic wasting disease.

In another aspect, the disclosure provides a method of reducing prion protein expression in a subject in need thereof by administering a therapeutically effective amount of an siRNA molecule or pharmaceutical composition of any of the preceding aspects or embodiments of the disclosure to the CNS of the subject.

In some embodiments, the subject has been identified as having a high-penetrance PRNP mutation.

In some embodiments, the high-penetrance mutation is E200K with respect to the amino acid sequence of human PrP (UNIPROT™ Accession No. P04156-1). In some embodiments, the high-penetrance mutation is D178N with respect to the amino acid sequence of human PrP (UNIPROT™ Accession No. P04156-1). In some embodiments, the high-penetrance mutation is P102L with respect to the amino acid sequence of human PrP (UNIPROT™ Accession No. P04156-1). In some embodiments, the high-penetrance mutation is 6-OPRI with respect to the amino acid sequence of human PrP (UNIPROT™ Accession No. P04156-1). In some embodiments, the high-penetrance mutation is 5-OPRI with respect to the amino acid sequence of human PrP (UNIPROT™ Accession No. P04156-1). In some embodiments, the high-penetrance mutation is A117V with respect to the amino acid sequence of human PrP (UNIPROT™ Accession No. P04156-1). In some embodiments, the high-penetrance mutation is P105L with respect to the amino acid sequence of human PrP (UNIPROT™ Accession No. P04156-1). In some embodiments, the high-penetrance mutation is one of those described in Minikel et al., Neurology 93(2):e125-e134 (2019).

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “nucleoside” refers to a molecule made up of a heterocyclic base and its sugar.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like.

Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that are comprised of a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems.

Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula -O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows: 100 multiplied by (the fraction X/Y) where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software.

Those skilled in the art can determine appropriate parameters for aligning sequences, including any algorithms needed to achieve maximal alignment over the full length of the sequences being compared.

For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of PRNP, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “high penetrance,” as used herein in reference to a genetic mutation, refers to a mutation in which there is a high likelihood that a subject having the mutation will exhibit a phenotype associated with that mutation. Specifically, when used in reference to a PRNP mutation, the term may refer to E200K, D178N, P102L, 6—OPRI, 5—OPRI, Al17V, or P105L with respect to the amino acid sequence of human PrP (UNIPROT™ Accession No. P04156-1). Additional examples of high-penetrance mutations are those described in Minikel et al., Neurology 93(2):e125-e134 (2019), the disclosure of which is incorporated herein by reference.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted.

Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C═C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C═CH and —C═C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂-.

A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H3N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human) that receives treatment for a prion disease. Examples of subjects and patients may also include those diagnosed with a specific prion disease including, but not limited to, Creutzfeldt-Jakob disease, fatal familial insomnia, Gerstmann-Straussler-Scheinker Syndrome, kuru, scrapie, bovine spongiform encephalopathy, and chronic wasting disease. In some embodiments, the terms “subject” and patients may refer to an organism that has a high-penetrance PRNP mutation.

As used herein, the term “prion” or “PrP” refers to any protein encoded by the PRNP gene including, but not limited to, the normal cellular isoform (PrP^{C}), or the disease-causing isoform (PrP^{SC}) As used herein, the term “prion disease” refers to any disease or condition in an organism, the pathogenesis of which involves a prion protein of the organism. Prion diseases include, but are not limited to, Creutzfeldt-Jakob disease, fatal familial insomnia, Gerstmann-Straussler-Scheinker Syndrome, kuru, scrapie, bovine spongiform encephalopathy, and chronic wasting disease. In general, prion diseases are caused by misfolding of the cellular isoform (PrP^{C}) of the prion protein encoded by PRNP.

The misfolded protein (PrP^{SC}) triggers the disease. The disease can propagate by PrP^{SC} inducing the misfolding of PrP^{C}.

As used herein, the term “PRNP” refers to the gene encoding the prion protein, including any native PRNP gene from any source. The term encompasses “full-length,” unprocessed PRNP as well as any form of PRNP that results from processing in the cell. The term also encompasses naturally occurring variants of PRNP, e.g., splice variants or allelic variants. The nucleic acid sequence of an exemplary PRNP gene is shown in European Nucleotide Archive (ENA) Accession No. M13899.1. The amino acid sequence of an exemplary protein encoded by a PRNP gene is shown in UNIPROT™ Accession No. P04156-1.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on analgesics; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, prion disease, e.g., Creutzfeldt-Jakob disease, fatal familial insomnia, or Gerstmann-Straussler-Scheinker Syndrome. The terms may also be used in the context of a subject having a high-penetrance mutation of PRNP. For example, clinical benefits in the context of a subject administered an siRNA molecule or siRNA composition of the disclosure include, without limitation, a reduction of: symptoms of a prion disease, wild type PRNP transcripts, mutant PRNP transcripts, variant PRNP transcripts, splice isoforms of PRNP transcripts, and/or overexpressed PRNP transcripts thereof (relative to a healthy subject).

### DETAILED DESCRIPTION

The present disclosure provides compositions of small interfering RNA (siRNA) molecules with sequence homology to a prion protein (PRNP) gene and methods for administering said siRNA molecules to the central nervous system of a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference by degrading mRNA with a complementary nucleotide sequence, thus preventing the translation of the target gene.

The siRNA molecules of the disclosure may exhibit, for example, robust gene-specific suppression of PRNP, relative to other human genes.

For example, the siRNA molecules of the disclosure may feature an antisense strand having a nucleic acid sequence that is complementary to a region of a PRNP mRNA transcript having the nucleic acid sequence of any one of SEQ ID NOs: 493-738. The degree of complementarity of the antisense strand to the region of the PRNP mRNA transcript may be sufficient for the antisense strand to anneal over the full length of the region of the PRNP mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%,74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the PRNP mRNA transcript.

In some embodiments, the siRNA molecules of the disclosure feature an antisense strand having the nucleic acid sequence of any one of SEQ ID NOs: 1-246, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%,74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-246.

In some embodiments, the siRNA molecules of the disclosure feature a sense strand having the nucleic acid sequence of any one of SEQ ID NOs: 247-492, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%,74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 247-492.

Exemplary siRNA molecules of the disclosure are those shown in Table 1, below. Table 1 summarizes the antisense strands, sense strands, and corresponding regions of a PRNP mRNA transcript that are targeted by each antisense strand.

TABLE 1
Nucleotide sequences for gene-specific PRNP-targeting siRNA

| | | | | Targeting | mRNA |
|---|---|---|---|---|---|
| Antisense | Antisense | Sense | Sense | Region | Targeting |
| SEQ ID NO. | Sequence | SEQ ID NO. | Sequence | SEQ ID NO. | Region |
| 1 | UGACGUGGC | 247 | UCAAUGAGC | 493 | UACAGUCAA |
| | UCAUUGACU | | CACGUCA | | UGAGCCACG |
| | GUA | | | | UCA |
| 2 | UGAACACUU | 248 | CUGAUGCAA | 494 | AUCAACUGA |
| | GCAUCAGUU | | GUGUUCA | | UGCAAGUGU |
| | GAU | | | | UCA |
| 3 | UAGUUAAAG | 249 | CUGCACCCU | 495 | CAGCGCUGC |
| | GGUGCAGC | | UUAACUA | | ACCCUUUAA |
| | GCUG | | | | CUU |
| 4 | UGCGACUGG | 250 | CUCCGAGCC | 496 | CUGUCCUCC |
| | CUCGGAGGA | | AGUCGCA | | GAGCCAGUC |
| | CAG | | | | GCU |
| 5 | UAGGAGAGG | 251 | AGCUUCUCC | 497 | CCGCGAGCU |
| | AGAAGCUCG | | UCUCCUA | | UCUCCUCUC |
| | CGG | | | | CUC |
| 6 | UUGAGGAGA | 252 | CUUCUCCUC | 498 | GCGAGCUUC |
| | GGAGAAGCU | | UCCUCAA | | UCCUCUCCU |
| | CGC | | | | CAC |
| 7 | UGUCGUGAG | 253 | UCCUCUCCU | 499 | GCUUCUCCU |
| | GAGAGGAGA | | CACGACA | | CUCCUCACG |
| | AGC | | | | ACC |
| 8 | UACUGUGGG | 254 | GGUGGCACC | 500 | AAGGAGGUG |
| | UGCCACCUC | | CACAGUA | | GCACCCACA |
| | CUU | | | | GUC |
| 9 | UUGGCUUAC | 255 | AAGCCGAGU | 501 | GGAACAAGC |
| | UCGGCUUGU | | AAGCCAA | | CGAGUAAGC |
| | UCC | | | | CAA |
| 10 | UUCCCAGCA | 256 | GGCUACAUG | 502 | UUGGCGGC |
| | UGUAGCCGC | | CUGGGAA | | UACAUGCUG |
| | CAA | | | | GGAA |
| 11 | UCGAAAUGU | 257 | CCAUCAUAC | 503 | CAGGCCCAU |
| | AUGAUGGGC | | AUUUCGA | | CAUACAUUU |
| | CUG | | | | CGG |
| 12 | UAUAGUCAC | 258 | UUCGGCAGU | 504 | UACAUUUCG |
| | UGCCGAAAU | | GACUAUA | | GCAGUGACU |
| | GUA | | | | AUG |
| 13 | UCCUCAUAG | 259 | GCAGUGACU | 505 | UUUCGGCAG |
| | UCACUGCCG | | AUGAGGA | | UGACUAUGA |
| | AAA | | | | GGA |
| 14 | UGUCCUCAU | 260 | AGUGACUAU | 506 | UCGGCAGUG |
| | AGUCACUGC | | GAGGACA | | ACUAUGAGG |
| | CGA | | | | ACC |
| 15 | UAACGGUCC | 261 | ACUAUGAGG | 507 | CAGUGACUA |
| | UCAUAGUCA | | ACCGUUA | | UGAGGACCG |
| | CUG | | | | UUA |
| 16 | UACGAUAGU | 262 | GACCGUUAC | 508 | AUGAGGACC |
| | AACGGUCCU | | UAUCGUA | | GUUACUAUC |
| | CAU | | | | GUG |
| 17 | UGUGCAUGU | 263 | CGUGAAAAC | 509 | ACUAUCGUG |
| | UUUCACGAU | | AUGCACA | | AAAACAUGC |
| | AGU | | | | ACC |
| 18 | UGGGUAACG | 264 | CAUGCACCG | 510 | GAAAACAUG |
| | GUGCAUGUU | | UUACCCA | | CACCGUUAC |
| | UUC | | | | CCC |
| 19 | UCCUGUAGU | 265 | CAAGUGUAC | 511 | CCAACCAAG |
| | ACACUUGGU | | UACAGGA | | UGUACUACA |
| | UGG | | | | GGC |
| 20 | UGCCUGUAG | 266 | AAGUGUACU | 512 | CAACCAAGU |
| | UACACUUGG | | ACAGGCA | | GUACUACAG |
| | UUG | | | | GCC |
| 21 | UAUCCAUGG | 267 | UACAGGCCC | 513 | UGUACUACA |
| | GCCUGUAGU | | AUGGAUA | | GGCCCAUGG |
| | ACA | | | | AUG |
| 22 | UCUCAUCCA | 268 | AGGCCCAUG | 514 | ACUACAGGC |
| | UGGGCCUG | | GAUGAGA | | CCAUGGAUG |
| | UAGU | | | | AGU |
| 23 | UACUCAUCC | 269 | GGCCCAUGG | 515 | CUACAGGCC |
| | AUGGGCCUG | | AUGAGUA | | CAUGGAUGA |
| | UAG | | | | GUA |
| 24 | UGCUGUACU | 270 | AUGGAUGAG | 516 | GGCCCAUGG |
| | CAUCCAUGG | | UACAGCA | | AUGAGUACA |
| | GCC | | | | GCA |
| 25 | UCUGGUUGC | 271 | GAGUACAGC | 517 | UGGAUGAGU |
| | UGUACUCAU | | AACCAGA | | ACAGCAACC |
| | CCA | | | | AGA |
| 26 | UGUUCUGGU | 272 | UACAGCAAC | 518 | AUGAGUACA |
| | UGCUGUACU | | CAGAACA | | GCAACCAGA |
| | CAU | | | | ACA |
| 27 | UGUUGUUCU | 273 | AGCAACCAG | 519 | AGUACAGCA |
| | GGUUGCUG | | AACAACA | | ACCAGAACA |
| | UACU | | | | ACU |
| 28 | UGCAGUCGU | 274 | UUUGUGCAC | 520 | ACAACUUUG |
| | GCACAAAGU | | GACUGCA | | UGCACGACU |
| | UGU | | | | GCG |
| 29 | UUUGACGCA | 275 | GCACGACUG | 521 | UUUGUGCAC |
| | GUCGUGCAC | | CGUCAAA | | GACUGCGUC |
| | AAA | | | | AAU |
| 30 | UAUUGACGC | 276 | CACGACUGC | 522 | UUGUGCACG |
| | AGUCGUGCA | | GUCAAUA | | ACUGCGUCA |
| | CAA | | | | AUA |
| 31 | UUAUUGACG | 277 | ACGACUGCG | 523 | UGUGCACGA |
| | CAGUCGUGC | | UCAAUAA | | CUGCGUCAA |
| | ACA | | | | UAU |
| 32 | UAUAUUGAC | 278 | CGACUGCGU | 524 | GUGCACGAC |
| | GCAGUCGUG | | CAAUAUA | | UGCGUCAAU |
| | CAC | | | | AUC |
| 33 | UGAUAUUGA | 279 | GACUGCGUC | 525 | UGCACGACU |
| | CGCAGUCGU | | AAUAUCA | | GCGUCAAUA |
| | GCA | | | | UCA |
| 34 | UUGAUAUUG | 280 | ACUGCGUCA | 526 | GCACGACUG |
| | ACGCAGUCG | | AUAUCAA | | CGUCAAUAU |
| | UGC | | | | CAC |
| 35 | UGCUUGAUU | 281 | AUAUCACAA | 527 | CGUCAAUAU |
| | GUGAUAUUG | | UCAAGCA | | CACAAUCAA |
| | ACG | | | | GCA |
| 36 | UCUGCUUGA | 282 | AUCACAAUC | 528 | UCAAUAUCA |
| | UUGUGAUAU | | AAGCAGA | | CAAUCAAGC |
| | UGA | | | | AGC |
| 37 | UGCUGCUUG | 283 | UCACAAUCA | 529 | CAAUAUCAC |
| | AUUGUGAUA | | AGCAGCA | | AAUCAAGCA |
| | UUG | | | | GCA |
| 38 | UGUGCUGCU | 284 | ACAAUCAAG | 530 | AUAUCACAA |
| | UGAUUGUGA | | CAGCACA | | UCAAGCAGC |
| | UAU | | | | ACA |
| 39 | UGUGACCGU | 285 | GCAGCACAC | 531 | AUCAAGCAG |
| | GUGCUGCUU | | GGUCACA | | CACACGGUC |
| | GAU | | | | ACC |
| 40 | UGGUGACCG | 286 | CAGCACACG | 532 | UCAAGCAGC |
| | UGUGCUGCU | | GUCACCA | | ACACGGUCA |
| | UGA | | | | CCA |
| 41 | UGUGGUGAC | 287 | GCACACGGU | 533 | AAGCAGCAC |
| | CGUGUGCU | | CACCACA | | ACGGUCACC |
| | GCUU | | | | ACA |
| 42 | UGUUGUGG | 288 | CACGGUCAC | 534 | CAGCACACG |
| | UGACCGUGU | | CACAACA | | GUCACCACA |
| | GCUG | | | | ACC |
| 43 | UCCUUGGUG | 289 | CCACAACCA | 535 | GGUCACCAC |
| | GUUGUGGU | | CCAAGGA | | AACCACCAA |
| | GACC | | | | GGG |
| 44 | UGGUCUCG | 290 | AACUUCACC | 536 | GGGAGAACU |
| | GUGAAGUUC | | GAGACCA | | UCACCGAGA |
| | UCCC | | | | CCG |
| 45 | UCGCGCUCC | 291 | AGAUGAUGG | 537 | CGUUAAGAU |
| | AUCAUCUUA | | AGCGCGA | | GAUGGAGCG |
| | ACG | | | | CGU |
| 46 | UCCACGCGC | 292 | UGAUGGAGC | 538 | UAAGAUGAU |
| | UCCAUCAUC | | GCGUGGA | | GGAGCGCG |
| | UUA | | | | UGGU |
| 47 | UCUGCUCAA | 293 | CGCGUGGU | 539 | UGGAGCGC |
| | CCACGCGCU | | UGAGCAGA | | GUGGUUGA |
| | CCA | | | | GCAGA |
| 48 | UAUGCUCGA | 294 | GAGAGGAUC | 540 | UACCAGAGA |
| | UCCUCUCUG | | GAGCAUA | | GGAUCGAGC |
| | GUA | | | | AUG |
| 49 | UAGAAGAGG | 295 | GCAUGGUCC | 541 | AUCGAGCAU |
| | ACCAUGCUC | | UCUUCUA | | GGUCCUCUU |
| | GAU | | | | CUC |
| 50 | UGGAGAAGA | 296 | AUGGUCCUC | 542 | CGAGCAUGG |
| | GGACCAUGC | | UUCUCCA | | UCCUCUUCU |
| | UCG | | | | CCU |
| 51 | UGGAGGAUC | 297 | CACCUGUGA | 543 | CUCUCCACC |
| | ACAGGUGGA | | UCCUCCA | | UGUGAUCCU |
| | GAG | | | | CCU |
| 52 | UAGAGAUCA | 298 | AUCCUCCUG | 544 | CUGUGAUCC |
| | GGAGGAUCA | | AUCUCUA | | UCCUGAUCU |
| | CAG | | | | CUU |
| 53 | UGGAAAGAG | 299 | UCCUGAUCU | 545 | GAUCCUCCU |
| | AUCAGGAGG | | CUUUCCA | | GAUCUCUUU |
| | AUC | | | | CCU |
| 54 | UUCAGGAAG | 300 | UCCUCAUCU | 546 | CUCUUUCCU |
| | AUGAGGAAA | | UCCUGAA | | CAUCUUCCU |
| | GAG | | | | GAU |
| 55 | UGAAGACCU | 301 | AUGAGGAAG | 547 | GUGGGAUGA |
| | UCCUCAUCC | | GUCUUCA | | GGAAGGUCU |
| | CAC | | | | UCC |
| 56 | UAAAGAUGG | 302 | GUUUUCACC | 548 | UUCCUGUUU |
| | UGAAAACAG | | AUCUUUA | | UCACCAUCU |
| | GAA | | | | UUC |
| 57 | UGAAAGAUG | 303 | UUUUCACCA | 549 | UCCUGUUUU |
| | GUGAAAACA | | UCUUUCA | | CACCAUCUU |
| | GGA | | | | UCU |
| 58 | UUAGAAAGA | 304 | UUCACCAUC | 550 | CUGUUUUCA |
| | UGGUGAAAA | | UUUCUAA | | CCAUCUUUC |
| | CAG | | | | UAA |
| 59 | UAGAUUAGA | 305 | CCAUCUUUC | 551 | UUUCACCAU |
| | AAGAUGGUG | | UAAUCUA | | CUUUCUAAU |
| | AAA | | | | CUU |
| 60 | UCCUAUCCG | 306 | UUUGUCCCG | 552 | CUCUCUUUG |
| | GGACAAAGA | | GAUAGGA | | UCCCGGAUA |
| | GAG | | | | GGC |
| 61 | UAUUAGCCU | 307 | CCCGGAUAG | 553 | UUUGUCCCG |
| | AUCCGGGAC | | GCUAAUA | | GAUAGGCUA |
| | AAA | | | | AUC |
| 62 | UUGAUUAGC | 308 | CGGAUAGGC | 554 | UGUCCCGGA |
| | CUAUCCGGG | | UAAUCAA | | UAGGCUAAU |
| | ACA | | | | CAA |
| 63 | UUUGAUUAG | 309 | GGAUAGGCU | 555 | GUCCCGGAU |
| | CCUAUCCGG | | AAUCAAA | | AGGCUAAUC |
| | GAC | | | | AAU |
| 64 | UUAUGUUUU | 310 | GCACUGGAA | 556 | GAUGGGCAC |
| | CCAGUGCCC | | AACAUAA | | UGGAAAACA |
| | AUC | | | | UAG |
| 65 | UUUACUGGC | 311 | AUGCCAGGC | 557 | UGCUAAUGC |
| | CUGGCAUUA | | CAGUAAA | | CAGGCCAGU |
| | GCA | | | | AAA |
| 66 | UCUUUUACU | 312 | CCAGGCCAG | 558 | UAAUGCCAG |
| | GGCCUGGCA | | UAAAAGA | | GCCAGUAAA |
| | UUA | | | | AGU |
| 67 | UACCAAUGG | 313 | CAAAUAACC | 559 | AACAGCAAA |
| | UUAUUUGCU | | AUUGGUA | | UAACCAUUG |
| | GUU | | | | GUU |
| 68 | UAUAAGUCC | 314 | UUAAUCUGG | 560 | AUUGGUUAA |
| | AGAUUAACC | | ACUUAUA | | UCUGGACUU |
| | AAU | | | | AUU |
| 69 | UUGUUUUAG | 315 | GUUGAGGCU | 561 | AACAGGUUG |
| | CCUCAACCU | | AAAACAA | | AGGCUAAAA |
| | GUU | | | | CAA |
| 70 | UAGAUUUGU | 316 | GGCUAAAAC | 562 | GUUGAGGCU |
| | UUUAGCCUC | | AAAUCUA | | AAAACAAAU |
| | AAC | | | | CUC |
| 71 | UGAGAUUUG | 317 | GCUAAAACA | 563 | UUGAGGCUA |
| | UUUUAGCCU | | AAUCUCA | | AAACAAAUC |
| | CAA | | | | UCA |
| 72 | UUGAGAUUU | 318 | CUAAAACAA | 564 | UGAGGCUAA |
| | GUUUUAGCC | | AUCUCAA | | AACAAAUCU |
| | UCA | | | | CAG |
| 73 | UCUGAGAUU | 319 | UAAAACAAA | 565 | GAGGCUAAA |
| | UGUUUUAGC | | UCUCAGA | | ACAAAUCUC |
| | CUC | | | | AGA |
| 74 | UUCUGAGAU | 320 | AAAACAAAU | 566 | AGGCUAAAA |
| | UUGUUUUAG | | CUCAGAA | | CAAAUCUCA |
| | CCU | | | | GAA |
| 75 | UUCAGACUG | 321 | CUCAGAACA | 567 | CAAAUCUCA |
| | UUCUGAGAU | | GUCUGAA | | GAACAGUCU |
| | UUG | | | | GAA |
| 76 | UAAGGUAUU | 322 | AGUCUGAAA | 568 | AGAACAGUC |
| | UCAGACUGU | | UACCUUA | | UGAAAUACC |
| | UCU | | | | UUU |
| 77 | UUGAAGGAG | 323 | CCUCUGGCU | 569 | GGAUACCUC |
| | CCAGAGGUA | | CCUUCAA | | UGGCUCCUU |
| | UCC | | | | CAG |
| 78 | UCUGAAGGA | 324 | CUCUGGCUC | 570 | GAUACCUCU |
| | GCCAGAGGU | | CUUCAGA | | GGCUCCUUC |
| | AUC | | | | AGC |
| 79 | UGCUGAAGG | 325 | UCUGGCUCC | 571 | AUACCUCUG |
| | AGCCAGAGG | | UUCAGCA | | GCUCCUUCA |
| | UAU | | | | GCA |
| 80 | UAGAUAGGG | 326 | ACUAAUGCC | 572 | AGUAUACUA |
| | CAUUAGUAU | | CUAUCUA | | AUGCCCUAU |
| | ACU | | | | CUU |
| 81 | UAAGAUAGG | 327 | CUAAUGCCC | 573 | GUAUACUAA |
| | GCAUUAGUA | | UAUCUUA | | UGCCCUAUC |
| | UAC | | | | UUA |
| 82 | UAAAUCUCU | 328 | UCUUAGUAG | 574 | CCCUAUCUU |
| | ACUAAGAUA | | AGAUUUA | | AGUAGAGAU |
| | GGG | | | | UUC |
| 83 | UGAAAUCUC | 329 | CUUAGUAGA | 575 | CCUAUCUUA |
| | UACUAAGAU | | GAUUUCA | | GUAGAGAUU |
| | AGG | | | | UCA |
| 84 | UAUAGCUAU | 330 | AGAUUUCAU | 576 | AGUAGAGAU |
| | GAAAUCUCU | | AGCUAUA | | UUCAUAGCU |
| | ACU | | | | AUU |
| 85 | UUAAAUAGC | 33 | UUUCAUAGC | 577 | AGAGAUUUC |
| | UAUGAAAUC | | UAUUUAA | | AUAGCUAUU |
| | UCU | | | | UAG |
| 86 | UUCGGGUUU | 332 | UUUAAGAAA | 578 | UCCAUUUUA |
| | UCUUAAAAU | | ACCCGAA | | AGAAAACCC |
| | GGA | | | | GAC |
| 87 | UGUCGGGU | 333 | UUAAGAAAA | 579 | CCAUUUUAA |
| | UUUCUUAAA | | CCCGACA | | GAAAACCCG |
| | AUGG | | | | ACA |
| 88 | UUGGCCUCC | 334 | UUUGUUAGG | 580 | CCAGGUUUG |
| | UAACAAACC | | AGGCCAA | | UUAGGAGGC |
| | UGG | | | | CAC |
| 89 | UUAUCAUGU | 335 | GGAGGCCAC | 581 | UGUUAGGAG |
| | GGCCUCCUA | | AUGAUAA | | GCCACAUGA |
| | ACA | | | | UAC |
| 90 | UGUAUCAUG | 336 | GAGGCCACA | 582 | GUUAGGAGG |
| | UGGCCUCCU | | UGAUACA | | CCACAUGAU |
| | AAC | | | | ACU |
| 91 | UUAAGUAUC | 337 | GCCACAUGA | 583 | AGGAGGCCA |
| | AUGUGGCCU | | UACUUAA | | CAUGAUACU |
| | CCU | | | | UAU |
| 92 | UCCAAGAGC | 338 | AUUCUUAGC | 584 | UAGAGAUUC |
| | UAAGAAUCU | | UCUUGGA | | UUAGCUCUU |
| | CUA | | | | GGG |
| 93 | UGUUCUCGG | 339 | GUGUGUACC | 585 | GCUCUGUGU |
| | UACACACAG | | GAGAACA | | GUACCGAGA |
| | AGC | | | | ACU |
| 94 | UAUACUGUG | 340 | UACUUUUCA | 586 | UGUUUUACU |
| | AAAAGUAAA | | CAGUAUA | | UUUCACAGU |
| | ACA | | | | AUG |
| 95 | UACAAUAUU | 341 | AAGAGUAAA | 587 | UCAACAAGA |
| | UACUCUUGU | | UAUUGUA | | GUAAAUAUU |
| | UGA | | | | GUC |
| 96 | UAAUAUGUC | 342 | GCUAGAGGA | 588 | CUCUGGCUA |
| | CUCUAGCCA | | CAUAUUA | | GAGGACAUA |
| | GAG | | | | UUC |
| 97 | UGAAUAUGU | 343 | CUAGAGGAC | 589 | UCUGGCUAG |
| | CCUCUAGCC | | AUAUUCA | | AGGACAUAU |
| | AGA | | | | UCA |
| 98 | UACAGUUAU | 344 | AGUGAACAU | 590 | UUCACAGUG |
| | GUUCACUGU | | AACUGUA | | AACAUAACU |
| | GAA | | | | GUA |
| 99 | UUACAGUUA | 345 | GUGAACAUA | 591 | UCACAGUGA |
| | UGUUCACUG | | ACUGUAA | | ACAUAACUG |
| | UGA | | | | UAA |
| 100 | UAGAAGCCU | 346 | AUAUGAAAG | 592 | AACAUAUAU |
| | UUCAUAUAU | | GCUUCUA | | GAAAGGCUU |
| | GUU | | | | CUG |
| 101 | UUCAAGUCC | 347 | GCUUCUGG | 593 | GAAAGGCUU |
| | CAGAAGCCU | | GACUUGAA | | CUGGGACUU |
| | UUC | | | | GAA |
| 102 | UAUUUGAUU | 348 | GACUUGAAA | 594 | UCUGGGACU |
| | UCAAGUCCC | | UCAAAUA | | UGAAAUCAA |
| | AGA | | | | AUG |
| 103 | UAAACAUCU | 349 | CCAUUUUAG | 595 | ACCUCCCAU |
| | AAAAUGGGA | | AUGUUUA | | UUUAGAUGU |
| | GGU | | | | UUA |
| 104 | UAUAUAGGG | 350 | UAAAGGACC | 596 | AUGUUUAAA |
| | UCCUUUAAA | | CUAUAUA | | GGACCCUAU |
| | CAU | | | | AUG |
| 105 | UAAGAAAGG | 351 | UGGCAUUCC | 597 | AUAUGUGGC |
| | AAUGCCACA | | UUUCUUA | | AUUCCUUUC |
| | UAU | | | | UUU |
| 106 | UCCUAUAGU | 352 | UCUUUAAAC | 598 | UCCUUUCUU |
| | UUAAAGAAA | | UAUAGGA | | UAAACUAUA |
| | GGA | | | | GGU |
| 107 | UAGCUGCCU | 353 | GUAAUUAAG | 599 | UAUAGGUAA |
| | UAAUUACCU | | GCAGCUA | | UUAAGGCAG |
| | AUA | | | | CUG |
| 108 | UCAGCUGCC | 354 | UAAUUAAGG | 600 | AUAGGUAAU |
| | UUAAUUACC | | CAGCUGA | | UAAGGCAGC |
| | UAU | | | | UGA |
| 109 | UUUCAGCUG | 355 | AUUAAGGCA | 601 | AGGUAAUUA |
| | CCUUAAUUA | | GCUGAAA | | AGGCAGCUG |
| | CCU | | | | AAA |
| 110 | UACUUUUCA | 356 | AGGCAGCUG | 602 | AAUUAAGGC |
| | GCUGCCUUA | | AAAAGUA | | AGCUGAAAA |
| | AUU | | | | GUA |
| 111 | UUACUUUUC | 357 | GGCAGCUGA | 603 | AUUAAGGCA |
| | AGCUGCCUU | | AAAGUAA | | GCUGAAAAG |
| | AAU | | | | UAA |
| 112 | UUUACUUUU | 358 | GCAGCUGAA | 604 | UUAAGGCAG |
| | CAGCUGCCU | | AAGUAAA | | CUGAAAAGU |
| | UAA | | | | AAA |
| 113 | UUUUACUUU | 359 | CAGCUGAAA | 605 | UAAGGCAGC |
| | UCAGCUGCC | | AGUAAAA | | UGAAAAGUA |
| | UUA | | | | AAU |
| 114 | UAUUUACUU | 360 | AGCUGAAAA | 606 | AAGGCAGCU |
| | UUCAGCUGC | | GUAAAUA | | GAAAAGUAA |
| | CUU | | | | AUU |
| 115 | UGGCAAUUU | 361 | GAAAAGUAA | 607 | CAGCUGAAA |
| | ACUUUUCAG | | AUUGCCA | | AGUAAAUUG |
| | CUG | | | | CCU |
| 116 | UCUAGAAGG | 362 | UAAAUUGCC | 608 | AAAAGUAAA |
| | CAAUUUACU | | UUCUAGA | | UUGCCUUCU |
| | UUU | | | | AGA |
| 117 | UUCAGUGUC | 363 | CCUUCUAGA | 609 | AAUUGCCUU |
| | UAGAAGGCA | | CACUGAA | | CUAGACACU |
| | AUU | | | | GAA |
| 118 | UUUGCCUUC | 364 | AGACACUGA | 610 | CUUCUAGAC |
| | AGUGUCUAG | | AGGCAAA | | ACUGAAGGC |
| | AAG | | | | AAA |
| 119 | UACAAAGGA | 365 | GCAAAUCUC | 611 | UGAAGGCAA |
| | GAUUUGCCU | | CUUUGUA | | AUCUCCUUU |
| | UCA | | | | GUC |
| 120 | UAAUGGACA | 366 | UCUCCUUUG | 612 | GCAAAUCUC |
| | AAGGAGAUU | | UCCAUUA | | CUUUGUCCA |
| | UGC | | | | UUU |
| 121 | UUUUCCAGG | 367 | CCAUUUACC | 613 | UUUGUCCAU |
| | UAAAUGGAC | | UGGAAAA | | UUACCUGGA |
| | AAA | | | | AAC |
| 122 | UGGUUUCCA | 368 | AUUUACCUG | 614 | UGUCCAUUU |
| | GGUAAAUGG | | GAAACCA | | ACCUGGAAA |
| | ACA | | | | CCA |
| 123 | UUCUGGUUU | 369 | UACCUGGAA | 615 | CCAUUUACC |
| | CCAGGUAAA | | ACCAGAA | | UGGAAACCA |
| | UGG | | | | GAA |
| 124 | UCAAAAUCA | 370 | ACCAGAAUG | 616 | UGGAAACCA |
| | UUCUGGUUU | | AUUUUGA | | GAAUGAUUU |
| | CCA | | | | UGA |
| 125 | UAGCUCUCC | 371 | ACAUACAGG | 617 | UUUUGACAU |
| | UGUAUGUCA | | AGAGCUA | | ACAGGAGAG |
| | AAA | | | | CUG |
| 126 | UGCUUUCAC | 372 | UGCAGUUGU | 618 | AGAGCUGCA |
| | AACUGCAGC | | GAAAGCA | | GUUGUGAAA |
| | UCU | | | | GCA |
| 127 | UGAUGGUGC | 373 | UGUGAAAGC | 619 | GCAGUUGUG |
| | UUUCACAAC | | ACCAUCA | | AAAGCACCA |
| | UGC | | | | UCA |
| 128 | UCCUCUAUG | 374 | CCAUCAUCA | 620 | AAGCACCAU |
| | AUGAUGGUG | | UAGAGGA | | CAUCAUAGA |
| | CUU | | | | GGA |
| 129 | UCAUCCUCU | 375 | UCAUCAUAG | 621 | CACCAUCAU |
| | AUGAUGAUG | | AGGAUGA | | CAUAGAGGA |
| | GUG | | | | UGA |
| 130 | UUACAUCAU | 376 | AUAGAGGAU | 622 | UCAUCAUAG |
| | CCUCUAUGA | | GAUGUAA | | AGGAUGAUG |
| | UGA | | | | UAA |
| 131 | UUUCUUUGC | 377 | UCAGUGUGC | 623 | AAUGGUCAG |
| | ACACUGACC | | AAAGAAA | | UGUGCAAAG |
| | AUU | | | | AAA |
| 132 | UCUUUUCUU | 378 | GUGUGCAAA | 624 | GGUCAGUGU |
| | UGCACACUG | | GAAAAGA | | GCAAAGAAA |
| | ACC | | | | AGA |
| 133 | UUUCUUUUC | 379 | GUGCAAAGA | 625 | UCAGUGUGC |
| | UUUGCACAC | | AAAGAAA | | AAAGAAAAG |
| | UGA | | | | AAC |
| 134 | UCAGUUCUU | 380 | CAAAGAAAA | 626 | GUGUGCAAA |
| | UUCUUUGCA | | GAACUGA | | GAAAAGAAC |
| | CAC | | | | UGC |
| 135 | UGCAGUUCU | 381 | AAAGAAAAG | 627 | UGUGCAAAG |
| | UUUCUUUGC | | AACUGCA | | AAAAGAACU |
| | ACA | | | | GCU |
| 136 | UAGCAGUUC | 382 | AAGAAAAGA | 628 | GUGCAAAGA |
| | UUUUCUUUG | | ACUGCUA | | AAAGAACUG |
| | CAC | | | | CUU |
| 137 | UAAAUAAAG | 383 | UGCAUUUCU | 629 | CUGCUUGCA |
| | AAAUGCAAG | | UUAUUUA | | UUUCUUUAU |
| | CAG | | | | UUC |
| 138 | UGAAAUAAA | 384 | GCAUUUCUU | 630 | UGCUUGCAU |
| | GAAAUGCAA | | UAUUUCA | | UUCUUUAUU |
| | GCA | | | | UCU |
| 139 | UAGAAAUAA | 385 | CAUUUCUUU | 631 | GCUUGCAUU |
| | AGAAAUGCA | | AUUUCUA | | UCUUUAUUU |
| | AGC | | | | CUG |
| 140 | UAGAAAUAA | 386 | CAUUUCUUU | 632 | GCUUGCAUU |
| | AGAAAUGCA | | AUUUCUA | | UCUUUAUUU |
| | AGC | | | | CUG |
| 141 | UACAAUUAU | 387 | CUGUCUCAU | 633 | UAUUUCUGU |
| | GAGACAGAA | | AAUUGUA | | CUCAUAAUU |
| | AUA | | | | GUC |
| 142 | UUUGACAAU | 388 | UCUCAUAAU | 634 | UUCUGUCUC |
| | UAUGAGACA | | UGUCAAA | | AUAAUUGUC |
| | GAA | | | | AAA |
| 143 | UACUAUGAA | 389 | GGUCAAGUU | 635 | AAUUAGGUC |
| | CUUGACCUA | | CAUAGUA | | AAGUUCAUA |
| | AUU | | | | GUU |
| 144 | UAACUAUGA | 390 | GUCAAGUUC | 636 | AUUAGGUCA |
| | ACUUGACCU | | AUAGUUA | | AGUUCAUAG |
| | AAU | | | | UUU |
| 145 | UAAACUAUG | 391 | UCAAGUUCA | 637 | UUAGGUCAA |
| | AACUUGACC | | UAGUUUA | | GUUCAUAGU |
| | UAA | | | | UUC |
| 146 | UGAAACUAU | 392 | CAAGUUCAU | 638 | UAGGUCAAG |
| | GAACUUGAC | | AGUUUCA | | UUCAUAGUU |
| | CUA | | | | UCU |
| 147 | UAGAAACUA | 393 | AAGUUCAUA | 639 | AGGUCAAGU |
| | UGAACUUGA | | GUUUCUA | | UCAUAGUUU |
| | CCU | | | | CUG |
| 148 | UCAGAAACU | 394 | AGUUCAUAG | 640 | GGUCAAGUU |
| | AUGAACUUG | | UUUCUGA | | CAUAGUUUC |
| | ACC | | | | UGU |
| 149 | UCAAUUACA | 395 | UAGUUUCUG | 641 | GUUCAUAGU |
| | GAAACUAUG | | UAAUUGA | | UUCUGUAAU |
| | AAC | | | | UGG |
| 150 | UAAAGCCAA | 396 | UCUGUAAUU | 642 | UAGUUUCUG |
| | UUACAGAAA | | GGCUUUA | | UAAUUGGCU |
| | CUA | | | | UUU |
| 151 | UUCAAAAGC | 397 | GUAAUUGGC | 643 | UUUCUGUAA |
| | CAAUUACAG | | UUUUGAA | | UUGGCUUUU |
| | AAA | | | | GAA |
| 152 | UGAUUCAAA | 398 | AUUGGCUUU | 644 | CUGUAAUUG |
| | AGCCAAUUA | | UGAAUCA | | GCUUUUGAA |
| | CAG | | | | UCA |
| 153 | UUGAUUCAA | 399 | UUGGCUUUU | 645 | UGUAAUUGG |
| | AAGCCAAUU | | GAAUCAA | | CUUUUGAAU |
| | ACA | | | | CAA |
| 154 | UUUCUUUGA | 400 | UUUUGAAUC | 646 | UUGGCUUUU |
| | UUCAAAAGC | | AAAGAAA | | GAAUCAAAG |
| | CAA | | | | AAU |
| 155 | UCUAUUCUU | 401 | UGAAUCAAA | 647 | GCUUUUGAA |
| | UGAUUCAAA | | GAAUAGA | | UCAAAGAAU |
| | AGC | | | | AGG |
| 156 | UCCUAUUCU | 402 | GAAUCAAAG | 648 | CUUUUGAAU |
| | UUGAUUCAA | | AAUAGGA | | CAAAGAAUA |
| | AAG | | | | GGG |
| 157 | UCUCCCUAU | 403 | UCAAAGAAU | 649 | UUGAAUCAA |
| | UCUUUGAUU | | AGGGAGA | | AGAAUAGGG |
| | CAA | | | | AGA |
| 158 | UUAGAUUGU | 404 | UAGGGAGAC | 650 | AAGAAUAGG |
| | CUCCCUAUU | | AAUCUAA | | GAGACAAUC |
| | CUU | | | | UAA |
| 159 | UUCUGUCAU | 405 | GUUGGAGAU | 651 | CUUAGGUUG |
| | CUCCAACCU | | GACAGAA | | GAGAUGACA |
| | AAG | | | | GAA |
| 160 | UUUCUGUCA | 406 | UUGGAGAUG | 652 | UUAGGUUGG |
| | UCUCCAACC | | ACAGAAA | | AGAUGACAG |
| | UAA | | | | AAA |
| 161 | UUUUCUGUC | 407 | UGGAGAUGA | 653 | UAGGUUGGA |
| | AUCUCCAAC | | CAGAAAA | | GAUGACAGA |
| | CUA | | | | AAU |
| 162 | UAUAUUUCU | 408 | AGAUGACAG | 654 | GUUGGAGAU |
| | GUCAUCUCC | | AAAUAUA | | GACAGAAAU |
| | AAC | | | | AUG |
| 163 | UUUCCACUU | 409 | UGAUUUGAA | 655 | AUGAUUGAU |
| | CAAAUCAAU | | GUGGAAA | | UUGAAGUGG |
| | CAU | | | | AAA |
| 164 | UUUUCCACU | 410 | GAUUUGAAG | 656 | UGAUUGAUU |
| | UCAAAUCAA | | UGGAAAA | | UGAAGUGGA |
| | UCA | | | | AAA |
| 165 | UAUCAAACA | 411 | CCUGAAUUG | 657 | UAUUCCCUG |
| | AUUCAGGGA | | UUUGAUA | | AAUUGUUUG |
| | AUA | | | | AUA |
| 166 | UGGUGACAA | 412 | UUUGAUAUU | 658 | AAUUGUUUG |
| | UAUCAAACA | | GUCACCA | | AUAUUGUCA |
| | AUU | | | | CCU |
| 167 | UGCUAGGUG | 413 | AUAUUGUCA | 659 | GUUUGAUAU |
| | ACAAUAUCA | | CCUAGCA | | UGUCACCUA |
| | AAC | | | | GCA |
| 168 | UCUGCUAGG | 414 | AUUGUCACC | 660 | UUGAUAUUG |
| | UGACAAUAU | | UAGCAGA | | UCACCUAGC |
| | CAA | | | | AGA |
| 169 | UAUAUCUGC | 415 | UCACCUAGC | 661 | UAUUGUCAC |
| | UAGGUGACA | | AGAUAUA | | CUAGCAGAU |
| | AUA | | | | AUG |
| 170 | UUACAUAUC | 416 | CCUAGCAGA | 662 | UGUCACCUA |
| | UGCUAGGUG | | UAUGUAA | | GCAGAUAUG |
| | ACA | | | | UAU |
| 171 | UAAAAGUAA | 417 | AUAUGUAUU | 663 | AGCAGAUAU |
| | UACAUAUCU | | ACUUUUA | | GUAUUACUU |
| | GCU | | | | UUC |
| 172 | UCAAUAAUA | 418 | GCAAUGUUA | 664 | UUUCUGCAA |
| | ACAUUGCAG | | UUAUUGA | | UGUUAUUAU |
| | AAA | | | | UGG |
| 173 | UCCAAUAAU | 419 | CAAUGUUAU | 665 | UUCUGCAAU |
| | AACAUUGCA | | UAUUGGA | | GUUAUUAUU |
| | GAA | | | | GGC |
| 174 | UGCAAGCCA | 420 | UUAUUAUUG | 666 | CAAUGUUAU |
| | AUAAUAACA | | GCUUGCA | | UAUUGGCUU |
| | UUG | | | | GCA |
| 175 | UCAAAGUGC | 421 | UUGGCUUGC | 667 | UAUUAUUGG |
| | AAGCCAAUA | | ACUUUGA | | CUUGCACUU |
| | AUA | | | | UGU |
| 176 | UCACAAAGU | 422 | GGCUUGCAC | 668 | UUAUUGGCU |
| | GCAAGCCAA | | UUUGUGA | | UGCACUUUG |
| | UAA | | | | UGA |
| 177 | UAAUACUCA | 423 | CACUUUGUG | 669 | GCUUGCACU |
| | CAAAGUGCA | | AGUAUUA | | UUGUGAGUA |
| | AGC | | | | UUC |
| 178 | UGAAUACUC | 424 | ACUUUGUGA | 670 | CUUGCACUU |
| | ACAAAGUGC | | GUAUUCA | | UGUGAGUAU |
| | AAG | | | | UCU |
| 179 | UAGAAUACU | 425 | CUUUGUGAG | 671 | UUGCACUUU |
| | CACAAAGUG | | UAUUCUA | | GUGAGUAUU |
| | CAA | | | | CUA |
| 180 | UUAGAAUAC | 426 | UUUGUGAGU | 672 | UGCACUUUG |
| | UCACAAAGU | | AUUCUAA | | UGAGUAUUC |
| | GCA | | | | UAU |
| 181 | UUAGAAUAC | 427 | UUUGUGAGU | 673 | UGCACUUUG |
| | UCACAAAGU | | AUUCUAA | | UGAGUAUUC |
| | GCA | | | | UAU |
| 182 | UAUAGAAUA | 428 | UUGUGAGUA | 674 | GCACUUUGU |
| | CUCACAAAG | | UUCUAUA | | GAGUAUUCU |
| | UGC | | | | AUG |
| 183 | UACAUAGAA | 429 | GUGAGUAUU | 675 | ACUUUGUGA |
| | UACUCACAA | | CUAUGUA | | GUAUUCUAU |
| | AGU | | | | GUA |
| 184 | UGUCUGUCC | 430 | UUGCAUAGG | 676 | AUAUAUUGC |
| | UAUGCAAUA | | ACAGACA | | AUAGGACAG |
| | UAU | | | | ACU |
| 185 | UAAGUCUGU | 431 | GCAUAGGAC | 677 | AUAUUGCAU |
| | CCUAUGCAA | | AGACUUA | | AGGACAGAC |
| | UAU | | | | UUA |
| 186 | UUAAGUCUG | 432 | CAUAGGACA | 678 | UAUUGCAUA |
| | UCCUAUGCA | | GACUUAA | | GGACAGACU |
| | AUA | | | | UAG |
| 187 | UCUAAGUCU | 433 | AUAGGACAG | 679 | AUUGCAUAG |
| | GUCCUAUGC | | ACUUAGA | | GACAGACUU |
| | AAU | | | | AGG |
| 188 | UUCCUAAGU | 434 | AGGACAGAC | 680 | UGCAUAGGA |
| | CUGUCCUAU | | UUAGGAA | | CAGACUUAG |
| | GCA | | | | GAG |
| 189 | UAAACUCCU | 435 | CAGACUUAG | 681 | UAGGACAGA |
| | AAGUCUGUC | | GAGUUUA | | CUUAGGAGU |
| | CUA | | | | UUU |
| 190 | UACUGCUCU | 436 | UUUGUUUAG | 682 | GGAGUUUUG |
| | AAACAAAAC | | AGCAGUA | | UUUAGAGCA |
| | UCC | | | | GUU |
| 191 | UAGAUGUUA | 437 | GAGCAGUUA | 683 | GUUUAGAGC |
| | ACUGCUCUA | | ACAUCUA | | AGUUAACAU |
| | AAC | | | | CUG |
| 192 | UCAGAUGUU | 438 | AGCAGUUAA | 684 | UUUAGAGCA |
| | AACUGCUCU | | CAUCUGA | | GUUAACAUC |
| | AAA | | | | UGA |
| 193 | UUCAGAUGU | 439 | GCAGUUAAC | 685 | UUAGAGCAG |
| | UAACUGCUC | | AUCUGAA | | UUAACAUCU |
| | UAA | | | | GAA |
| 194 | UUUCAGAUG | 440 | CAGUUAACA | 686 | UAGAGCAGU |
| | UUAACUGCU | | UCUGAAA | | UAACAUCUG |
| | CUA | | | | AAG |
| 195 | UCUUCAGAU | 441 | AGUUAACAU | 687 | AGAGCAGUU |
| | GUUAACUGC | | CUGAAGA | | AACAUCUGA |
| | UCU | | | | AGU |
| 196 | UUUAGACAC | 442 | AUCUGAAGU | 688 | UUAACAUCU |
| | UUCAGAUGU | | GUCUAAA | | GAAGUGUCU |
| | UAA | | | | AAU |
| 197 | UGCAUUAGA | 443 | UGAAGUGUC | 689 | ACAUCUGAA |
| | CACUUCAGA | | UAAUGCA | | GUGUCUAAU |
| | UGU | | | | GCA |
| 198 | UAAAAGUUA | 444 | AAUGCAUUA | 690 | UGUCUAAUG |
| | AUGCAUUAG | | ACUUUUA | | CAUUAACUU |
| | ACA | | | | UUG |
| 199 | UAGUACCUU | 445 | CUUUUGUAA | 691 | AUUAACUUU |
| | ACAAAAGUU | | GGUACUA | | UGUAAGGUA |
| | AAU | | | | CUG |
| 200 | UCAGUACCU | 446 | UUUUGUAAG | 692 | UUAACUUUU |
| | UACAAAAGU | | GUACUGA | | GUAAGGUAC |
| | UAA | | | | UGA |
| 201 | UUCAGUACC | 447 | UUUGUAAGG | 693 | UAACUUUUG |
| | UUACAAAAG | | UACUGAA | | UAAGGUACU |
| | UUA | | | | GAA |
| 202 | UUUCAGUAC | 448 | UUGUAAGGU | 694 | AACUUUUGU |
| | CUUACAAAA | | ACUGAAA | | AAGGUACUG |
| | GUU | | | | AAU |
| 203 | UAUUCAGUA | 449 | UGUAAGGUA | 695 | ACUUUUGUA |
| | CCUUACAAA | | CUGAAUA | | AGGUACUGA |
| | AGU | | | | AUA |
| 204 | UUAAGUAUU | 450 | GGUACUGAA | 696 | UGUAAGGUA |
| | CAGUACCUU | | UACUUAA | | CUGAAUACU |
| | ACA | | | | UAA |
| 205 | UUCCCACAU | 451 | ACUUAAUAU | 697 | UGAAUACUU |
| | AUUAAGUAU | | GUGGGAA | | AAUAUGUGG |
| | UCA | | | | GAA |
| 206 | UUUGUAAGC | 452 | UCCUUAGGC | 698 | CGUGGUCCU |
| | CUAAGGACC | | UUACAAA | | UAGGCUUAC |
| | ACG | | | | AAU |
| 207 | UCAUGAAAC | 453 | CUGAAUCGU | 699 | GUGCACUGA |
| | GAUUCAGUG | | UUCAUGA | | AUCGUUUCA |
| | CAC | | | | UGU |
| 208 | UAUUCUUAC | 454 | GUUUCAUGU | 700 | GAAUCGUUU |
| | AUGAAACGA | | AAGAAUA | | CAUGUAAGA |
| | UUC | | | | AUC |
| 209 | UUUGGAUUC | 455 | CAUGUAAGA | 701 | CGUUUCAUG |
| | UUACAUGAA | | AUCCAAA | | UAAGAAUCC |
| | ACG | | | | AAA |
| 210 | UCACUUUGG | 456 | UAAGAAUCC | 702 | UCAUGUAAG |
| | AUUCUUACA | | AAAGUGA | | AAUCCAAAG |
| | UGA | | | | UGG |
| 211 | UAAUGGUGU | 457 | AAAGUGGAC | 703 | AAUCCAAAG |
| | CCACUUUGG | | ACCAUUA | | UGGACACCA |
| | AUU | | | | UUA |
| 212 | UUUAAUGGU | 458 | AGUGGACAC | 704 | UCCAAAGUG |
| | GUCCACUUU | | CAUUAAA | | GACACCAUU |
| | GGA | | | | AAC |
| 213 | UAAAGACCU | 459 | CAUUAACAG | 705 | GACACCAUU |
| | GUUAAUGGU | | GUCUUUA | | AACAGGUCU |
| | GUC | | | | UUG |
| 214 | UUGCAUAUU | 460 | UCUUUGAAA | 706 | ACAGGUCUU |
| | UCAAAGACC | | UAUGCAA | | UGAAAUAUG |
| | UGU | | | | CAU |
| 215 | UAACAUGCA | 461 | GUAACUUUG | 707 | UAUUUGUAA |
| | AAGUUACAA | | CAUGUUA | | CUUUGCAUG |
| | AUA | | | | UUC |
| 216 | UGAACAUGC | 462 | UAACUUUGC | 708 | AUUUGUAAC |
| | AAAGUUACA | | AUGUUCA | | UUUGCAUGU |
| | AAU | | | | UCU |
| 217 | UAGAACAUG | 463 | AACUUUGCA | 709 | UUUGUAACU |
| | CAAAGUUAC | | UGUUCUA | | UUGCAUGUU |
| | AAA | | | | CUU |
| 218 | UAAGAACAU | 464 | ACUUUGCAU | 710 | UUGUAACUU |
| | GCAAAGUUA | | GUUCUUA | | UGCAUGUUC |
| | CAA | | | | UUG |
| 219 | UCAAGAACA | 465 | CUUUGCAUG | 711 | UGUAACUUU |
| | UGCAAAGUU | | UUCUUGA | | GCAUGUUCU |
| | ACA | | | | UGU |
| 220 | UCAAGAACA | 466 | CUUUGCAUG | 712 | UGUAACUUU |
| | UGCAAAGUU | | UUCUUGA | | GCAUGUUCU |
| | ACA | | | | UGU |
| 221 | UACAAGAAC | 467 | UUUGCAUGU | 713 | GUAACUUUG |
| | AUGCAAAGU | | UCUUGUA | | CAUGUUCUU |
| | UAC | | | | GUU |
| 222 | UAACAAGAA | 468 | UUGCAUGUU | 714 | UAACUUUGC |
| | CAUGCAAAG | | CUUGUUA | | AUGUUCUUG |
| | UUA | | | | UUU |
| 223 | UAAACAAGA | 469 | UGCAUGUUC | 715 | AACUUUGCA |
| | ACAUGCAAA | | UUGUUUA | | UGUUCUUGU |
| | GUU | | | | UUU |
| 224 | UAAAACAAG | 470 | GCAUGUUCU | 716 | ACUUUGCAU |
| | AACAUGCAA | | UGUUUUA | | GUUCUUGUU |
| | AGU | | | | UUG |
| 225 | UCAAAACAA | 471 | CAUGUUCUU | 717 | CUUUGCAUG |
| | GAACAUGCA | | GUUUUGA | | UUCUUGUUU |
| | AAG | | | | UGU |
| 226 | UCAAAACAA | 472 | CAUGUUCUU | 718 | CUUUGCAUG |
| | GAACAUGCA | | GUUUUGA | | UUCUUGUUU |
| | AAG | | | | UGU |
| 227 | UACAAAACA | 473 | AUGUUCUUG | 719 | UUUGCAUGU |
| | AGAACAUGC | | UUUUGUA | | UCUUGUUUU |
| | AAA | | | | GUU |
| 228 | UGUUUAAUU | 474 | UGACUGAAA | 720 | AUAUCUGAC |
| | UCAGUCAGA | | UUAAACA | | UGAAAUUAA |
| | UAU | | | | ACG |
| 229 | UGCUCGUUU | 475 | UGAAAUUAA | 721 | CUGACUGAA |
| | AAUUUCAGU | | ACGAGCA | | AUUAAACGA |
| | CAG | | | | GCG |
| 230 | UCGCUCGUU | 476 | GAAAUUAAA | 722 | UGACUGAAA |
| | UAAUUUCAG | | CGAGCGA | | UUAAACGAG |
| | UCA | | | | CGA |
| 231 | UUCGCUCGU | 477 | AAAUUAAAC | 723 | GACUGAAAU |
| | UUAAUUUCA | | GAGCGAA | | UAAACGAGC |
| | GUC | | | | GAA |
| 232 | UUUCGCUCG | 478 | AAUUAAACG | 724 | ACUGAAAUU |
| | UUUAAUUUC | | AGCGAAA | | AAACGAGCG |
| | AGU | | | | AAG |
| 233 | UCUUCGCUC | 479 | AUUAAACGA | 725 | CUGAAAUUA |
| | GUUUAAUUU | | GCGAAGA | | AACGAGCGA |
| | CAG | | | | AGA |
| 234 | UACGGUAGU | 480 | GACCGCUAC | 726 | GGGAGGACC |
| | AGCGGUCCU | | UACCGUA | | GCUACUACC |
| | CCC | | | | GUG |
| 235 | UGUGGUGAC | 481 | GCACACGGU | 727 | AAGCAGCAC |
| | CGUGUGCU | | CACCACA | | ACGGUCACC |
| | GCUU | | | | ACC |
| 236 | UGGAGAUGA | 482 | AUCCUCCUC | 728 | CUGUCAUCC |
| | GGAGGAUGA | | AUCUCCA | | UCCUCAUCU |
| | CAG | | | | CCU |
| 237 | UUUAUUCAC | 483 | UGGUCUAGU | 729 | UGUUUUGGU |
| | UAGACCAAA | | GAAUAAA | | CUAGUGAAU |
| | ACA | | | | AAA |
| 238 | UGUAUUUAU | 484 | CUAGUGAAU | 730 | UUGGUCUAG |
| | UCACUAGAC | | AAAUACA | | UGAAUAAAU |
| | CAA | | | | ACU |
| 239 | UGCUCUAGC | 485 | UGGGAUAGC | 731 | CCACCUGGG |
| | UAUCCCAGG | | UAGAGCA | | AUAGCUAGA |
| | UGG | | | | GCA |
| 240 | UUUGCUUUC | 486 | UGACGUUGA | 732 | CCUACUGAC |
| | AACGUCAGU | | AAGCAAA | | GUUGAAAGC |
| | AGG | | | | AAA |
| 241 | UUCUAGUGC | 487 | UCCCAGGGC | 733 | UUCAUUCCC |
| | CCUGGGAAU | | ACUAGAA | | AGGGCACUA |
| | GAA | | | | GAA |
| 242 | UAGCUUUUG | 488 | CAAUUAUCA | 734 | UCUCACAAU |
| | AUAAUUGUG | | AAAGCUA | | UAUCAAAAG |
| | AGA | | | | CUA |
| 243 | UAGUACAGA | 489 | CUAUGUUUC | 735 | CUGCCCUAU |
| | AACAUAGGG | | UGUACUA | | GUUUCUGUA |
| | CAG | | | | CUU |
| 244 | UAGCAGAAU | 490 | GAAAGAAAU | 736 | AGUGGGAAA |
| | UUCUUUCCC | | UCUGCUA | | GAAAUUCUG |
| | ACU | | | | CUA |
| 245 | UAAAGAAGA | 49 | GCUUAUGUC | 737 | UUCAAGCUU |
| | CAUAAGCUU | | UUCUUUA | | AUGUCUUCU |
| | GAA | | | | UUU |
| 246 | UAAAAUACA | 492 | CUUUGCAUG | 738 | UGUAACUUU |
| | UGCAAAGUU | | UAUUUUA | | GCAUGUAUU |
| | ACA | | | | UUG |

Iₙ Table 1, SEQ ID NOs 493-725 are human RNA sequences. In Table 1, SEQ ID Nos 726-738 are mouse RNA sequences. In Table 1, the human sequences have an mRNA accession number of NM_000311. In Table 1, the mouse sequences have an mRNA accession number of NM_011170.

siRNA Structure

The small interfering RNA (siRNA) molecules of the disclosure may be in the form of a single-stranded (ss) or double-stranded (ds) RNA structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest small interfering RNAs (siRNAs) consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNA interference (RNAi) activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides. 2′ Sugar Modifications The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C₂ to C10 alkenyl and alkynyl. In some embodiments, the modification includes a 2′—O-methyl (2′—O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′—O—CH₂CH₂OCH₃, also known as 2′—O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′—O-dimethylamino-ethoxy-ethyl or 2′-DMAEOE), i.e., 2′—O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure.

As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. Several tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

Internucleoside Linkage Modifications Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Pattern Modifications of siRNA Molecules The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

A-B-(A′)ⱼ-C-P²-D-P¹-(C′-P¹)ₖ-C′ Formula I;

wherein A is represented by the formula C-P¹-D-P¹; each A′, independently, is represented by the formula C-P²-D-P²; B is represented by the formula C-P²-D-P²-D-P²-D-P²; each C, independently, is a 2′—O-methyl (2′—O-Me) ribonucleoside; each C′, independently, is a 2′—O-Me ribonucleoside or a 2′-fluoro (2′-F) ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction

A-B-(A′)ⱼ-C-P²-D-P¹-(C-P¹)ₖ-C′ Formula II;

wherein A is represented by the formula C-P¹-D-P¹; each A′, independently, is represented by the formula C-P²-D-P²; B is represented by the formula C-P²-D-P²-D-P²-D-P²; each C, independently, is a 2′—O-methyl (2′—O-Me) ribonucleoside; each C′, independently, is a 2′—O-Me ribonucleoside or a 2′-fluoro (2′-F) ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

E-(A′)ₘ-F Formula III;

wherein E is represented by the formula (C-P¹)₂; F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction

A-(A′)ⱼ-C-P²-B-(C-P¹)ₖ-C′ Formula IV;

wherein A is represented by the formula C-P¹-D-P¹; each A′, independently, is represented by the formula C-P²-D-P²; B is represented by the formula D-P¹-C-P¹-D-P¹; each C, independently, is a 2′—O-Me ribonucleoside; each C′, independently, is a 2′—O-Me ribonucleoside or a 2′-F ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

E-(A′)ₘ-C-P²-F Formula V;

wherein E is represented by the formula (C-P¹)₂; F is represented by the formula D-P¹-C-P¹-C, D-P²-C-P²-C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

A-Bⱼ-E-Bₖ-E-F-Gi-D-P¹-C′ Formula VI;

wherein A is represented by the formula C-P¹-D-P¹; each B, independently, is represented by the formula C-P²; each C, independently, is a 2′—O-Me ribonucleoside; each C′, independently, is a 2′—O-Me ribonucleoside or a 2′-F ribonucleoside; each D, independently, is a 2′-F ribonucleoside; each E, independently, is represented by the formula D-P²-C-P²; F is represented by the formula D-P¹-C-P¹; each G, independently, is represented by the formula C-P¹; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and I is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and I is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction

H-Bₘ-Iₙ-A′-Bₒ-H-C Formula VII;

wherein A′ is represented by the formula C-P²-D-P²; each H, independently, is represented by the formula (C-P¹)₂; each I, independently, is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′—O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

Z-((A-P-)n(B-P-)ₘ)_{q};Formula VIII

wherein Z is a 5′ phosphorus stabilizing moiety; each A is, independently, a 2′—O-methyl (2′—O-Me) ribonucleoside; each B is, independently, a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

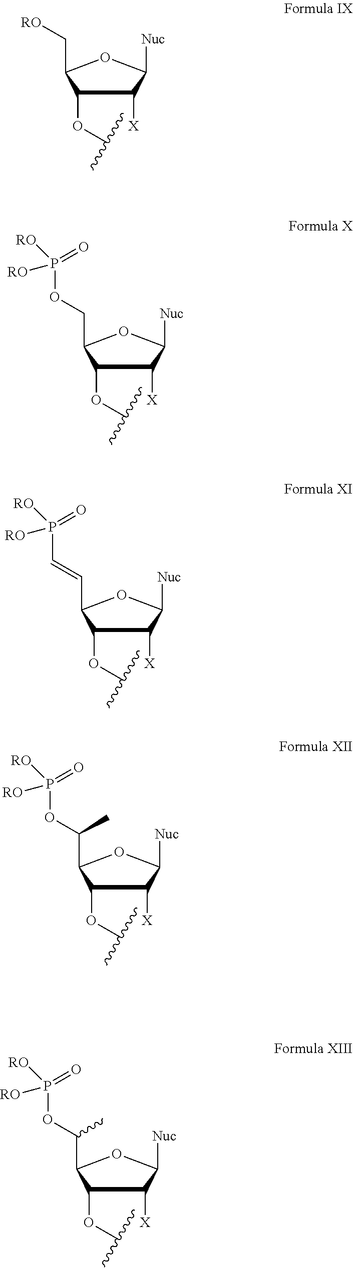

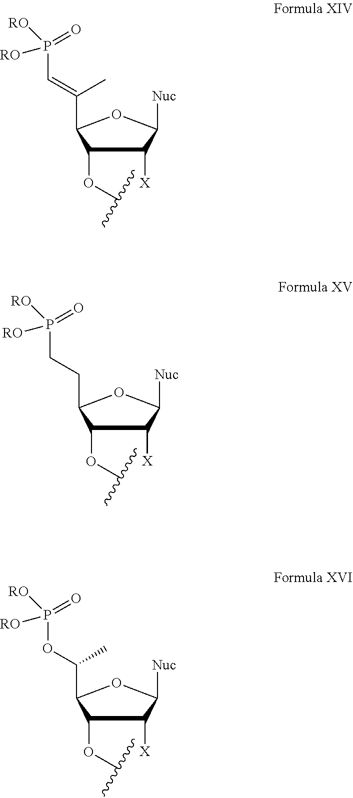

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substitued phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA-L-RNA Formula XVII | 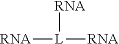 Formula XX | 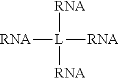 Formula XXIV |
| 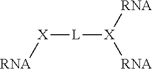 Formula XVIII | 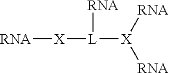 Formula XXI | 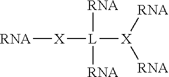 Formula XXV |
| 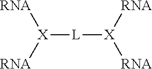 Formula XIX | 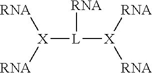 Formula XXII | 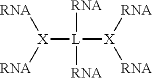 Formula XXVI |
| | 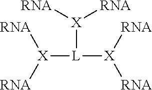 Formula XXIII | 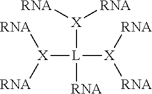 Formula XXVII |
| | | 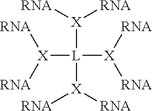 Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene linker (TEG).

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

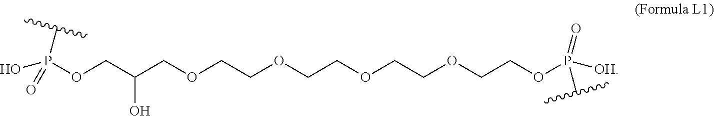 [SMILES: *P(=O)(O)OCCOCCOCCOCCOCC(O)COP(*)(=O)O]

In some embodiments, the linker has a structure of Formula L2:

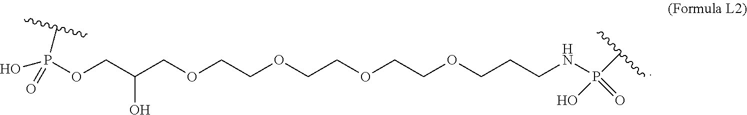 [SMILES: *P(=O)(O)NCCCOCCOCCOCCOCC(O)COP(*)(=O)O]

In some embodiments, the linker has a structure of Formula L3:

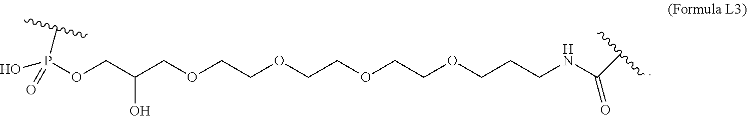 [SMILES: *C(=O)NCCCOCCOCCOCCOCC(O)COP(*)(=O)O]

In some embodiments, the linker has a structure of Formula L4:

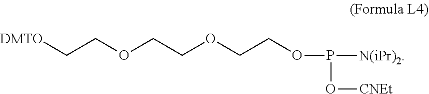

In some embodiments, the linker has a structure of Formula L5:

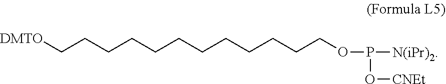

In some embodiments, the linker has a structure of Formula L6:

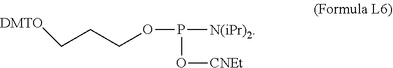

In some embodiments, the linker has a structure of Formula L7, as is shown below:

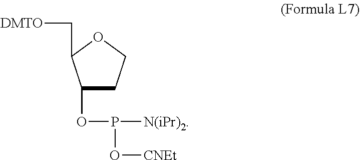

In some embodiments, the linker has a structure of Formula L8:

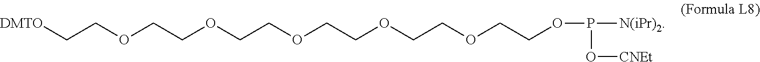

In some embodiments, the linker has a structure of Formula L9:

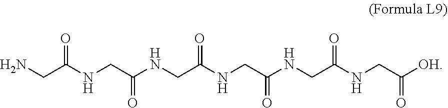 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The infectious agent of prion diseases appears to be composed exclusively of a protein. The infectious protein is an isoform of the cellular, PrP^{C}, PrP^{C} is expressed in healthy subjects, primarily in the nervous system. Misfolding of the prion protein into the PrP^{SC} isoform is a cause of degenerative brain disease, with symptoms that include ataxia, insomnia, and dementia. The misfolded isoform also induces the further misfolding of PrP^{C} into PrP^{SC}, thus propagating the disease. Prion diseases are fatal. The mechanism of the disease is largely the same regardless of the clinical subtype. Clinical subtypes of the disease in humans include, but are not limited to, Creutzfeldt-Jakob disease, fatal familial insomnia, Gerstmann-Straussler-Scheinker Syndrome. Clinical subtypes of prion diseases in non-human mammals include, but are not limited to, scrapie, bovine spongiform encephalopathy, and chronic wasting disease

The PRNP-targeting siRNA molecules of the disclosure may be delivered to a subject, for example, as a treatment for a prion disease. Furthermore, the siRNA molecules of the disclosure may also be delivered to a subject having a variant of the PRNP gene for which siRNA-mediated gene silencing of the PRNP variant gene reduces the expression level of PRNP transcript, thereby reducing the expression of prion protein. The siRNA molecules of the disclosure may also be delivered to a subject known to have a high-penetrance mutation of PRNP, such as E200K, D178N, P102L, 6—OPRI, 5—OPRI, A117V, or P105L with respect to the amino acid sequence of human PrP (UNIPROT™ Accession No. P04156-1). The siRNA molecules of the disclosure may also be delivered to a subject known to have a high-penetrance mutation of PRNP as described in Minikel et al., Neurology 93(2):e125-e134 (2019), the disclosure of which is incorporated herein by reference.

The disclosure provides methods of treating a subject by way of PRNP gene silencing with one or more of the small interfering RNA (siRNA) molecules described herein. The gene silencing may be performed in a subject to silence wild type PRNP transcripts, mutant PRNP transcripts, splice isoforms of PRNP transcripts, and/or overexpressed PRNP transcripts thereof, relative to a healthy subject. The method may include delivering to the CNS of the subject (e.g., a human) the siRNA molecules of the disclosure or a pharmaceutical composition containing the same by any appropriate route of administration (e.g., intrastriatal, intracerebroventricular, intrathecal injection, or by intra-cisterna magna injection by catheterization). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the small interfering RNA (siRNA) molecules disclosed herein are subjects in need of treatment for a prion disease. Subjects may be presymptomatic individuals who have been identified as having a high-penetrance PRNP mutation such as E200K, D178N, P102L, 6—OPRI, 5—OPRI, A117V, or P105L with respect to the amino acid sequence of human PrP (UNIPROT™ Accession No. P04156-1). The high-penetrance mutation of PRNP may also be one described in Minikel et al., Neurology 93(2):e125-e134 (2019). Additionally, subjects in need of treatment may be characterized as having a specific prion disorder. For example, the subject may be diagnosed with Creutzfeldt-Jakob disease, a degenerative brain disorder leading to dementia and death. The subject may be diagnosed with fatal familial insomnia, a degenerative brain disease characterized by inability to sleep, eventually leading to dementia and death. The subject may be diagnosed with Gerstmann-Straussler-Scheinker syndrome, a degenerative brain disorder characterized by progressive physical and mental deterioration leading to coma and death. A subject treated with an siRNA molecule of the disclosure may be a pre-symptomatic patient known to carry a high-penetrance PRNP mutation. Subjects that may be treated with the siRNA molecules disclosed herein may comprise, for example, humans, monkeys, rats, mice, pigs, and other mammals containing at least one orthologous copy of the prion protein (PRNP) gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS of the subject (e.g., by way of intrastriatal, intracerebroventricular, intrathecal injection or by intra-cisterna magna injection by catheterization).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, intrastriatally or by intra-cisterna magna injection by catheterization (e.g., injection into the caudate nucleus or putamen). A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna.

The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generating PRNP-Targeting siRNA Molecules

The small interfering RNA (siRNA) molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the prion protein gene (PRNP) mRNA target sequence, are shown below in Table 1, above. It is appreciated that one of skill in the art could anneal the antisense (AS) strand to the corresponding sense (S) strand to yield a ds-siRNA molecule. Alternatively, one of skill in the art could derive a ss-siRNA molecule using antisense strand only.

### Example 2. Optimizing PRNP-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences.

Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′—O-methyl (2′—O-Me), 2′-methoxyethoxy (2′—O—CH₂CH₂OCH₃, also known as 2′—O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′—O-dimethylamino-ethoxy-ethyl or 2′-DMAEOE), i.e., 2′—O—CH₂OCH₂N(CH₃)₂. Other possible 2′-modifications that can optimize the siRNA molecules of the disclosure include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C₂ to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, Iithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 3. Preparation and Administrating PRNP-Targeting siRNA Molecules

The small interfering RNA (siRNA) molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject's CNS that is tolerated by the siRNA compositions of the disclosure. Non-limiting examples of siRNA injections into the CNS include intrathecally, intracerebroventricularly, intrastriatally or intra-cisterna magna injection by catheterization (e.g., injection into the caudate nucleus or putamen). A physician having ordinary skill in the art can readily determine an effective route of administration.

### Example 4. Methods for the Treatment of Prion Diseases Using PRNP-Targeting siRNA Molecules

A subject in need of treatment for a prion disease, or a pre-symptomatic individual known to carry a high-penetrance PRNP mutation, is treated with a dosage of the small interfering RNA (siRNA) molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a therapeutic effect is achieved (e.g., a reduction in expression of prion protein (PRNP) mRNA). In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, intrastriatally or by intra-cisterna magna injection by way of catheterization (e.g., injection into the caudate nucleus or putamen). A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally or by intra-cisterna magna injection by way of catheterization) and condition at a rate tolerable to the patient until the subject has reached a maximum tolerated dose, or until the symptoms of the prion disease are ameliorated satisfactorily.

### Example 5. Methods for the Treatment of Exemplary Prion Diseases

The small interfering RNA (siRNA) molecules of the disclosure can be used for the treatment of specific prion disorders, such as those induced by gain-of-function PRNP gene variants. Non-limiting examples of clinical diagnoses suitable for treatment with the siRNA molecules of the disclosure include Creutzfeldt-Jakob disease, Gerstmann-Straussler-Scheinker Syndrome, or Fatal Familial Insomnia. Pre-symptomatic subjects known to have a high-penetrance PRNP mutation are also suitable for treatment with the siRNA molecules of the disclosure.

A subject with a condition of Creutzfeldt-Jakob disease, Gerstmann-Straussler-Scheinker Syndrome, or Fatal Familial Insomnia, or a pre-symptomatic individual known to have a high-penetrance PRNP mutation, is treated with a dosage of the siRNA molecule or composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a therapeutic effect is achieved (e.g., a reduction in expression of PRNP mRNA). In general, a suitable daily dose of one of one of the siRNA molecules of the disclosure will be an amount which is the lowest dose effective to produce a therapeutic effect. The ss- or ds-interfering RNA molecules of the disclosure may be administered by injection, e.g., intrathecally, intracerebroventricularly, intrastriatally or by intra-cisterna magna injection by way of catheterization (e.g., injection into the caudate nucleus or putamen). A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally or by intra-cisterna magna injection by way of catheterization) and condition at a rate tolerable to the patient until the subject has reached a maximum tolerated dose, or until the symptoms are ameliorated satisfactorily.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within a prion protein (PRNP) mRNA transcript having the nucleic acid sequence of any one of SEQ ID NOs: 493-738.

2. The siRNA molecule of claim 1, wherein the antisense strand has at least 70% complementarity to a region of 19, 20, 21, or more contiguous nucleobases within the region within the PRNP mRNA transcript having the nucleic acid sequence of any one of SEQ ID NOs: 493-738, optionally wherein the antisense strand has at least 70% complementarity to the PRNP mRNA transcript having the nucleic acid sequence of any one of SEQ ID NOs: 493-738.

3. The siRNA molecule of claim 2, wherein the antisense strand has at least 75% complementarity to a region of 21 contiguous nucleobases within the PRNP mRNA transcript having the nucleic acid sequence of any one of SEQ ID NOs: 493-738, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the PRNP mRNA transcript having the nucleic acid sequence of any one of SEQ ID Nos: 493-738.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the PRNP RNA transcript having the nucleic acid sequence of any one of SEQ ID NOs: 493-738.

5. The siRNA molecule of claim 4, wherein the antisense strand comprises from 10 to 30 contiguous nucleotides that are fully complementary to a contiguous polynucleotide segment of equal length within the region of the PRNP RNA transcript having the nucleic acid sequence of any one of SEQ ID NOs: 493-738.

6. The siRNA molecule of any claim 5, wherein the antisense strand comprises from 12 to 30 contiguous nucleotides that are fully complementary to a contiguous polynucleotide segment of equal length within the region of the PRNP RNA transcript having the nucleic acid sequence of any one of SEQ ID NOs: 493-738.

7. The siRNA molecule of claim 6, wherein the antisense strand comprises from 15 to 30 contiguous nucleotides that are fully complementary to a contiguous polynucleotide segment of equal length within the region of the PRNP RNA transcript having the nucleic acid sequence of any one of SEQ ID NOs: 493-738.

8. The siRNA molecule of claim 7, wherein the antisense strand comprises from 18 to 30 contiguous nucleotides that are fully complementary to a contiguous polynucleotide segment of equal length within the region of the PRNP RNA transcript having the nucleic acid sequence of any one of SEQ ID NOs: 493-738.

9. The siRNA molecule of claim 8, wherein the antisense strand comprises from 18 to 25 contiguous nucleotides that are fully complementary to a contiguous polynucleotide segment of equal length within the region of the PRNP RNA transcript having the nucleic acid sequence of any one of SEQ ID Nos: 493-738.

10. The siRNA molecule of claim 9 wherein the antisense strand comprises from 18 to 21 contiguous nucleotides that are fully complementary to a contiguous polynucleotide segment of equal length within the region of the PRNP RNA transcript having the nucleic acid sequence of any one of SEQ ID NOs: 493-738.

11. The siRNA molecule of claim 10, wherein the antisense strand comprises 21 contiguous nucleotides that are fully complementary to a contiguous polynucleotide segment of equal length within the region of the PRNP RNA transcript having the nucleic acid sequence of any one of SEQ ID NOs: 493-738.

12. The siRNA molecule of any one of claims 1-11, wherein the antisense strand comprises 9 or fewer nucleotide mismatches relative to the region of the PRNP RNA transcript having the nucleic acid sequence of any one of SEQ ID NOs: 493-738, optionally wherein the antisense strand comprises 8 or fewer, 7 or fewer, 6 or fewer, 5 or fewer, 4 or fewer, 3 or fewer, 2 or fewer, or only 1 mismatch relative to the region of the PRNP RNA transcript having the nucleic acid sequence of any one of SEQ ID NOs: 493-738.

13. The siRNA molecule of any one of claims 1-12, wherein the antisense strand has a nucleic acid sequence that is at least 85% identical to the nucleic acid sequence of any one of SEQ ID NOs: 1-246.

14. The siRNA molecule of any one of claims 1-13, wherein the antisense strand has a nucleic acid sequence that is at least 90% identical to the nucleic acid sequence of any one of SEQ ID NOs: 1-246.

15. The siRNA molecule of any one of claims 1-14, wherein the antisense strand has a nucleic acid sequence that is at least 95% identical to the nucleic acid sequence of SEQ ID NOs: 1-246, optionally wherein the antisense strand has a nucleic acid sequence that is at least 96%, 97%, 98%, or 99% identical to the nucleic acid sequence of any one of SEQ ID NOs: 1-246.

16. The siRNA molecule of claim 15, wherein the antisense strand has the nucleic acid sequence of any one of SEQ ID NOs: 1-246.

17. The siRNA molecule of any one of claims 1-16, wherein the sense strand has a nucleic acid sequence that is at least 85% identical to the nucleic acid sequence of any one of SEQ ID NOs: 247-492.

18. The siRNA molecule of any one of claims 1-17, wherein the sense strand has a nucleic acid sequence that is at least 90% identical to the nucleic acid sequence of any one of SEQ ID NOs: 247-492.

19. The siRNA molecule of any one of claims 1-18, wherein the sense strand has a nucleic acid sequence that is at least 95% identical to the nucleic acid sequence of SEQ ID NOs: 247-492, optionally wherein the sense strand has a nucleic acid sequence that is at least 96%, 97%, 98%, or 99% identical to the nucleic acid sequence of any one of SEQ ID NOs: 247-492.

20. The siRNA molecule of claim 19, wherein the sense strand has the nucleic acid sequence of any one of SEQ ID NOs: 247-492.

21. The siRNA molecule of any one of claims 1-20, wherein the antisense strand comprises a structure represented by Formula I, wherein Formula I is, in the 5′-to-3′ direction:
A-B-(A′)ⱼ-C-P²-D-P¹-(C′-P¹)ₖ-C′Formula I;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′—O-methyl (2′—O-Me) ribonucleoside;
each C′, independently, is a 2′—O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

22. The siRNA molecule of claim 21, wherein the antisense strand comprises a structure represented by Formula A1, wherein Formula A1 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′—O-Me ribonucleoside, B represents a 2′-F ribonucleoside, O represents a phosphodiester internucleoside linkage, and S represents a phosphorothioate internucleoside linkage.

23. The siRNA molecule of any one of claims 1-20, wherein the antisense strand comprises a structure represented by Formula II, wherein Formula II is, in the 5′-to-3′ direction:
A-B-(A′)ⱼ-C-P²-D-P¹-(C-P¹)ₖ-C′Formula II;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′—O-methyl (2′—O-Me) ribonucleoside;
each C′, independently, is a 2′—O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

24. The siRNA molecule of claim 23, wherein the antisense strand comprises a structure represented by Formula A2, wherein Formula A2 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′—O-Me ribonucleoside, B represents a 2′-F ribonucleoside, O represents a phosphodiester internucleoside linkage, and S represents a phosphorothioate internucleoside linkage.

25. The siRNA molecule of any one of claims 1-24, wherein the sense strand comprises a structure represented by Formula III, wherein Formula III is, in the 5′-to-3′ direction:
E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.

26. The siRNA molecule of claim 25, wherein the sense strand comprises a structure represented by Formula S1, wherein Formula S1 is, in the 5′-to-3′ direction:
A-S-A-S-A-O-B-O-A-O-B-O-A-O-B-O-A-O-B-O-A-O-A-O-A-O-B-S-A-S-A Formula S1;
wherein A represents a 2′—O-Me ribonucleoside, B represents a 2′-F ribonucleoside, O represents a phosphodiester internucleoside linkage, and S represents a phosphorothioate internucleoside linkage.

27. The siRNA molecule of claim 25, wherein the sense strand comprises a structure represented by Formula S2, wherein Formula S2 is, in the 5′-to-3′ direction:
A-S-A-S-A-O-B-O-A-O-B-O-A-O-B-O-A-O-B-O-A-O-A-O-A-O-B-O-A-O-A Formula S2;
wherein A represents a 2′—O-Me ribonucleoside, B represents a 2′-F ribonucleoside, O represents a phosphodiester internucleoside linkage, and S represents a phosphorothioate internucleoside linkage.

28. The siRNA molecule of claim 25, wherein the sense strand comprises a structure represented by Formula S3, wherein Formula S3 is, in the 5′-to-3′ direction:
A-S-A-S-A-O-B-O-A-O-B-O-A-O-B-O-A-O-B-O-A-O-A-O-A-O-B-S-A-S-B Formula S3;
wherein A represents a 2′—O-Me ribonucleoside, B represents a 2′-F ribonucleoside, O represents a phosphodiester internucleoside linkage, and S represents a phosphorothioate internucleoside linkage.

29. The siRNA molecule of claim 25, wherein the sense strand comprises a structure represented by Formula S4, wherein Formula S4 is, in the 5′-to-3′ direction:
A-S-A-S-A-O-B-O-A-O-B-O-A-O-B-O-A-O-B-O-A-O-A-O-A-O-B-O-A-O-B Formula S4;
wherein A represents a 2′—O-Me ribonucleoside, B represents a 2′-F ribonucleoside, O represents a phosphodiester internucleoside linkage, and S represents a phosphorothioate internucleoside linkage.

30. The siRNA molecule of any one of claims 1-20, wherein the antisense strand comprises a structure represented by Formula IV, wherein Formula IV is, in the 5′-to-3′ direction:
A-(A′)ⱼ-C-P²-B-(C-P¹)ₖ-C′ Formula IV;
wherein A is represented by the formula C-P¹-D-P¹;
each A′, independently, is represented by the formula C-P²-D-P²;
B is represented by the formula D-P¹-C-P¹-D-P¹;
each C, independently, is a 2′—O-Me ribonucleoside;
each C′, independently, is a 2′—O-Me ribonucleoside or a 2′-F ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

31. The siRNA molecule of claim 30, wherein the antisense strand comprises a structure represented by Formula A3, wherein Formula A3 is, in the 5′-to-3′ direction:
A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′—O-Me ribonucleoside, B represents a 2′-F ribonucleoside, O represents a phosphodiester internucleoside linkage, and S represents a phosphorothioate internucleoside linkage.

32. The siRNA molecule of any one of claims 1-24, 30, and 31, wherein the sense strand comprises a structure represented by Formula V, wherein Formula V is, in the 5′-to-3′ direction:
E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

33. The siRNA molecule of claim 32, wherein the sense strand comprises a structure represented by Formula S5, wherein Formula S5 is, in the 5′-to-3′ direction:
A-S-A-S-A-O-B-O-A-O-B-O-A-O-B-O-A-O-B-O-A-O-B-O-A-O-B-S-A-S-A Formula S5;
wherein A represents a 2′—O-Me ribonucleoside, B represents a 2′-F ribonucleoside, O represents a phosphodiester internucleoside linkage, and S represents a phosphorothioate internucleoside linkage.

34. The siRNA molecule of claim 32, wherein the sense strand comprises a structure represented by Formula S6, wherein Formula S6 is, in the 5′-to-3′ direction:
A-S-A-S-A-O-B-O-A-O-B-O-A-O-B-O-A-O-B-O-A-O-B-O-A-O-B-O-A-O-A Formula S6;
wherein A represents a 2′—O-Me ribonucleoside, B represents a 2′-F ribonucleoside, O represents a phosphodiester internucleoside linkage, and S represents a phosphorothioate internucleoside linkage.

35. The siRNA molecule of claim 32, wherein the sense strand comprises a structure represented by Formula S7, wherein Formula S7 is, in the 5′-to-3′ direction:
A-S-A-S-A-O-B-O-A-O-B-O-A-O-B-O-A-O-B-O-A-O-B-O-A-O-B-S-A-S-B Formula S7;
wherein A represents a 2′—O-Me ribonucleoside, B represents a 2′-F ribonucleoside, O represents a phosphodiester internucleoside linkage, and S represents a phosphorothioate internucleoside linkage.

36. The siRNA molecule of claim 32, wherein the sense strand comprises a structure represented by Formula S8, wherein Formula S8 is, in the 5′-to-3′ direction:
A-S-A-S-A-O-B-O-A-O-B-O-A-O-B-O-A-O-B-O-A-O-B-O-A-O-B-O-A-O-B Formula S8;
wherein A represents a 2′—O-Me ribonucleoside, B represents a 2′-F ribonucleoside, O represents a phosphodiester internucleoside linkage, and S represents a phosphorothioate internucleoside linkage.

37. The siRNA molecule of any one of claims 1-20, wherein the antisense strand comprises a structure represented by Formula VI, wherein Formula VI is, in the 5′-to-3′ direction:
A-Bⱼ-E-Bₖ-E-F-Gi-D-P¹-C′ Formula VI;
wherein A is represented by the formula C-P¹-D-P¹;
each B, independently, is represented by the formula C-P²;
each C, independently, is a 2′—O-Me ribonucleoside;
each C′, independently, is a 2′—O-Me ribonucleoside or a 2′-F ribonucleoside;
each D, independently, is a 2′-F ribonucleoside;
each E, independently, is represented by the formula D-P²-C-P²;
F is represented by the formula D-P¹-C-P¹;
each G, independently, is represented by the formula C-P¹;
each P¹ is, independently, a phosphorothioate internucleoside linkage;
each P² is, independently, a phosphodiester internucleoside linkage;
j is an integer from 1 to 7;
k is an integer from 1 to 7; and
l is an integer from 1 to 7.

38. The siRNA molecule of claim 37, wherein the antisense strand comprises a structure represented by Formula A4, wherein Formula A4 is, in the 5′-to-3′ direction:
A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′—O-Me ribonucleoside, B represents a 2′-F ribonucleoside, O represents a phosphodiester internucleoside linkage, and S represents a phosphorothioate internucleoside linkage.

39. The siRNA molecule of any one of claims 1-24, 30, 31, 37, and 38, wherein the sense strand comprises a structure represented by Formula VII, wherein Formula VII is, in the 5′-to-3′ direction:
H-Bₘ-Iₙ-A′-Bₒ-H-C Formula VII;
wherein A′ is represented by the formula C-P²-D-P²;
each H, independently, is represented by the formula (C-P¹)₂;
each I, independently, is represented by the formula (D-P²);
B, C, D, P¹ and P² are as defined in Formula VI;
m is an integer from 1 to 7;
n is an integer from 1 to 7; and
o is an integer from 1 to 7.

40. The siRNA molecule of claim 39, wherein the sense strand comprises a structure represented by Formula S9, wherein Formula S9 is, in the 5′-to-3′ direction:
A-S-A-S-A-O-A-O-A-O-B-O-B-O-B-O-A-O-B-O-A-O-A-O-A-O-A-S-A-S-A Formula S9;
wherein A represents a 2′—O-Me ribonucleoside, B represents a 2′-F ribonucleoside, O represents a phosphodiester internucleoside linkage, and S represents a phosphorothioate internucleoside linkage.

41. The siRNA molecule of any one of claims 1-40, wherein the antisense strand further comprises a 5′ phosphorus stabilizing moiety at the 5′ end of the antisense strand.

42. The siRNA molecule of any one of claims 1-41, wherein the sense strand further comprises a 5′ phosphorus stabilizing moiety at the 5′ end of the sense strand.

43. The siRNA molecule of claim 41 or 42, wherein each 5′ phosphorus stabilizing moiety is, independently, represented by any one of Formulas IX-XVI: 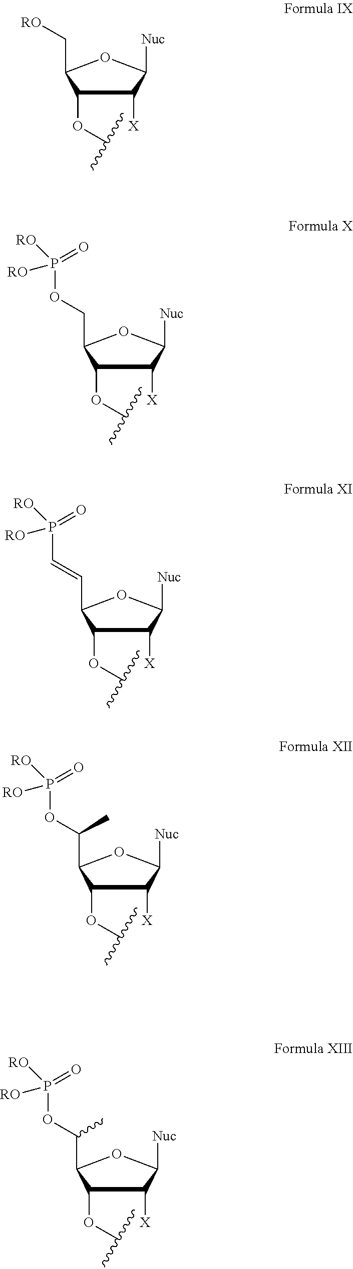 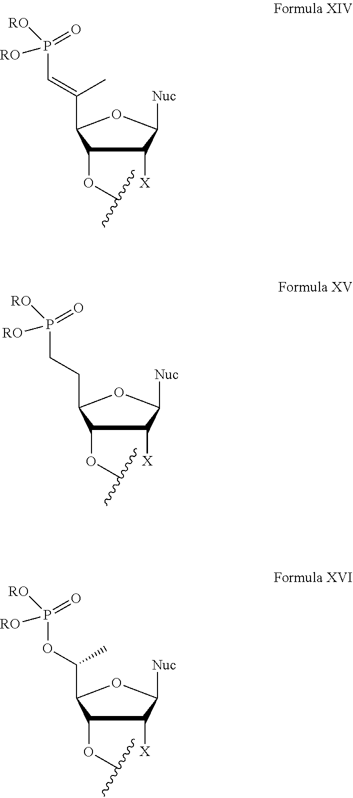
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, a cation, or hydrogen.

44. The siRNA molecule of claim 43, wherein the nucleobase is an adenine, uracil, guanine, thymine, or cytosine.

45. The siRNA molecule of any one of claims 41-44, wherein the 5′ phosphorus stabilizing moiety is (E)-vinylphosphonate represented by Formula XI.

46. The siRNA molecule of any one of claims 1-45, wherein the siRNA molecule further comprises a hydrophobic moiety at the 5′ or the 3′ end of the siRNA molecule.

47. The siRNA molecule of claim 46, wherein the hydrophobic moiety is selected from a group consisting of cholesterol, vitamin D, or tocopherol.

48. The siRNA molecule of any one of claims 1-47, wherein the length of the sense strand is between 10 and 30 nucleotides.

49. The siRNA molecule of claim 48, wherein the length of the sense strand is between 10 and 25 nucleotides.

50. The siRNA molecule of claim 49, wherein the length of the sense strand is between 12 and 25 nucleotides.

51. The siRNA molecule of claim 50, wherein the length of the sense strand is between 12 and 20 nucleotides.

52. The siRNA molecule of claim 51, wherein the length of the sense strand is between 12 and 19 nucleotides.

53. The siRNA molecule of claim 52, wherein the length of the sense strand is 15 nucleotides.

54. The siRNA molecule of claim 52, wherein the length of the sense strand is 16 nucleotides.

55. The siRNA molecule of claim 52, wherein the length of the sense strand is 18 nucleotides.

56. The siRNA molecule of any one of claims 1-55, wherein the length of the antisense strand is between 10 and 30 nucleotides.

57. The siRNA molecule of claim 56, wherein the length of the antisense strand is between 12 and 30 nucleotides.

58. The siRNA molecule of claim 57, wherein the length of the antisense strand is between 15 and 30 nucleotides.

59. The siRNA molecule of claim 58, wherein the length of the antisense strand is between 18 and 30 nucleotides.

60. The siRNA molecule of claim 59, wherein the length of the antisense strand is between 18 and 25 nucleotides.

61. The siRNA molecule of claim 60, wherein the length of the antisense strand is between 18 and 21 nucleotides.

62. The siRNA molecule of claim 61, wherein the length of the antisense strand is 18 nucleotides.

63. The siRNA molecule of claim 61, wherein the length of the antisense strand is 20 nucleotides.

64. The siRNA molecule of claim 61, wherein the length of the antisense strand is 21 nucleotides.

65. The siRNA molecule of any one of claims 1-64, wherein the siRNA molecule is a branched siRNA molecule.

66. The siRNA molecule of claim 65, wherein the branched siRNA molecule is di-branched, tri-branched, or tetra-branched.

67. The siRNA molecule of claim 66, wherein the siRNA molecule is a di-branched siRNA molecule, optionally wherein the di-branched siRNA molecule is represented by any one of Formulas XVII-XIX:
RNA-L-RNA Formula XVII; 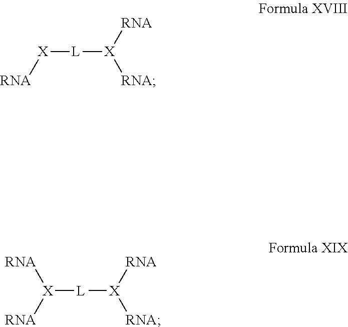
wherein each RNA is, independently, an siRNA molecule, L is a linker, and each X, independently, represents a branch point moiety.

68. The siRNA molecule of claim 66, wherein the siRNA molecule is a tri-branched siRNA molecule, optionally wherein the tri-branched siRNA molecule is represented by any one of Formulas XX-XXIII: 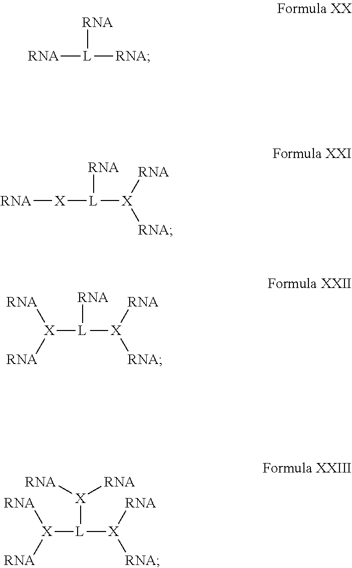
wherein each RNA is, independently, an siRNA molecule, L is a linker, and each X, independently, represents a branch point moiety.

69. The siRNA molecule of claim 66, wherein the siRNA molecule is a tetra-branched siRNA molecule, optionally wherein the tetra-branched siRNA molecule is represented by any one of Formulas XXIV-XXVIII: 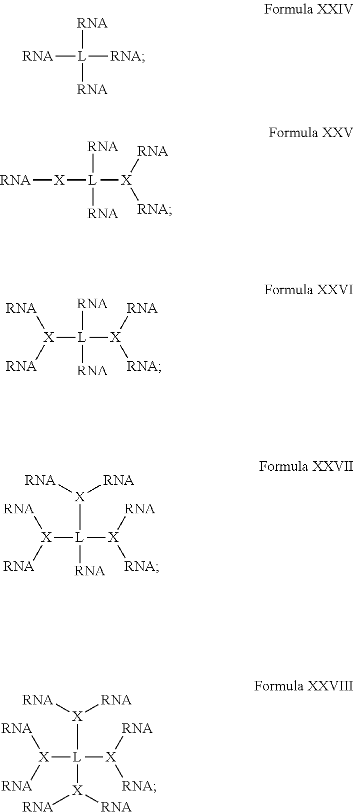
wherein each RNA is, independently, an siRNA molecule, L is a linker, and each X, independently, represents a branch point moiety.

70. The siRNA molecule of any one of claims 67-69, wherein the linker is selected from a group consisting of one or more contiguous subunits of an ethylene glycol, alkyl, carbohydrate, block copolymer, peptide, RNA, and DNA.

71. The siRNA molecule of claim 70, wherein the one or more contiguous subunits is 2 to 20 contiguous subunits.

72. A pharmaceutical composition comprising the siRNA molecule of any one of claims 1-71 and a pharmaceutically acceptable excipient, carrier, or diluent.

73. A method of delivering an siRNA molecule to the central nervous system (CNS) of a subject diagnosed as having a prion disease, or a pre-symptomatic individual identified as having a high-penetrance PRNP mutation associated with onset of a prion disease, the method comprising administering a therapeutically effective amount of the siRNA molecule of any one of claims 1-71 or the pharmaceutical composition of claim 72 to the subject.

74. A method of treating a prion disease in a subject in need thereof, the method comprising administering a therapeutically effective amount of the siRNA molecule of any one of claims 1-71 or the pharmaceutical composition of claim 72 to the subject.

75. The method of claim 73 or 74, wherein the prion disease is Creutzfeldt-Jakob Disease, Gerstmann-Straussler-Scheinker Syndrome, Fatal Familial Insomnia, kuru, scrapie, bovine spongiform encephalopathy, or chronic wasting disease.

76. A method of reducing prion protein expression in a subject in need thereof, the method comprising administering a therapeutically effective amount of the siRNA molecule of any one of claims 1-71 or the pharmaceutical composition of claim 72 to the CNS of the subject.

77. The method of any one of claims 73-76, wherein the subject has been identified as having a high-penetrance PRNP mutation

78. The method of claim 77, wherein the high-penetrance PRNP mutation is E200K, D178N, P102L, 6—OPRI, 5—OPRI, A117V, or P105L.

79. The method of any one of claims 73-78, wherein the siRNA molecule or the pharmaceutical composition is administered to the subject by way of intrastriatal, intracerebroventricular, or intrathecal injection.

80. The method of any one of claims 73-79, wherein the subject is a human.

81. A kit comprising the siRNA molecule of any one of claims 1-71, or the pharmaceutical composition of claim 72, and a package insert, wherein the package insert instructs a user of the kit to perform the method of any one of claims 73-80.


SEQUENCE LISTING (738 sequences)

SEQ ID NO: 1 - RNA, 21 nt, synthetic construct
  source 1..21: mol_type = other RNA
tgacgtggctcattgactgta

SEQ ID NO: 2 - RNA, 21 nt, synthetic construct
  source 1..21: mol_type = other RNA
tgaacacttgcatcagttgat

SEQ ID NO: 3 - RNA, 21 nt, synthetic construct
  source 1..21: mol_type = other RNA
tagttaaagggtgcagcgctg

SEQ ID NO: 4 - RNA, 21 nt, synthetic construct
  source 1..21: mol_type = other RNA
tgcgactggctcggaggacag

SEQ ID NO: 5 - RNA, 21 nt, synthetic construct
  source 1..21: mol_type = other RNA
taggagaggagaagctcgcgg

SEQ ID NO: 6 - RNA, 21 nt, synthetic construct
  source 1..21: mol_type = other RNA
ttgaggagaggagaagctcgc

SEQ ID NO: 7 - RNA, 21 nt, synthetic construct
  source 1..21: mol_type = other RNA
tgtcgtgaggagaggagaagc

SEQ ID NO: 8 - RNA, 21 nt, synthetic construct
  source 1..21: mol_type = other RNA
tactgtgggtgccacctcctt

SEQ ID NO: 9 - RNA, 21 nt, synthetic construct
  source 1..21: mol_type = other RNA
ttggcttactcggcttgttcc

SEQ ID NO: 10 - RNA, 21 nt, synthetic construct
  source 1..21: mol_type = other RNA
ttcccagcatgtagccgccaa

SEQ ID NO: 11 - RNA, 21 nt, synthetic construct
  source 1..21: mol_type = other RNA
tcgaaatgtatgatgggcctg

SEQ ID NO: 12 - RNA, 21 nt, synthetic construct
  source 1..21: mol_type = other RNA
tatagtcactgccgaaatgta

SEQ ID NO: 13 - RNA, 21 nt, synthetic construct
  source 1..21: mol_type = other RNA
tcctcatagtcactgccgaaa

SEQ ID NO: 14 - RNA, 21 nt, synthetic construct
  source 1..21: mol_type = other RNA
tgtcctcatagtcactgccga

SEQ ID NO: 15 - RNA, 21 nt, synthetic construct
  source 1..21: mol_type = other RNA
taacggtcctcatagtcactg

SEQ ID NO: 16 - RNA, 21 nt, synthetic construct
  source 1..21: mol_type = other RNA
tacgatagtaacggtcctcat

SEQ ID NO: 17 - RNA, 21 nt, synthetic construct
  source 1..21: mol_type = other RNA
tgtgcatgttttcacgatagt

SEQ ID NO: 18 - RNA, 21 nt, synthetic construct
  source 1..21: mol_type = other RNA
tgggtaacggtgcatgttttc

SEQ ID NO: 19 - RNA, 21 nt, synthetic construct
  source 1..21: mol_type = other RNA
tcctgtagtacacttggttgg

SEQ ID NO: 20 - RNA, 21 nt, synthetic construct
  source 1..21: mol_type = other RNA
tgcctgtagtacacttggttg

SEQ ID NO: 21 - RNA, 21 nt, synthetic construct
  source 1..21: mol_type = other RNA
tatccatgggcctgtagtaca

SEQ ID NO: 22 - RNA, 21 nt, synthetic construct
  source 1..21: mol_type = other RNA
tctcatccatgggcctgtagt

SEQ ID NO: 23 - RNA, 21 nt, synthetic construct
  source 1..21: mol_type = other RNA
tactcatccatgggcctgtag

SEQ ID NO: 24 - RNA, 21 nt, synthetic construct
  source 1..21: mol_type = other RNA
tgctgtactcatccatgggcc

SEQ ID NO: 25 - RNA, 21 nt, synthetic construct
  source 1..21: mol_type = other RNA
tctggttgctgtactcatcca

SEQ ID NO: 26 - RNA, 21 nt, synthetic construct
  source 1..21: mol_type = other RNA
tgttctggttgctgtactcat

SEQ ID NO: 27 - RNA, 21 nt, synthetic construct
  source 1..21: mol_type = other RNA
tgttgttctggttgctgtact

SEQ ID NO: 28 - RNA, 21 nt, synthetic construct
  source 1..21: mol_type = other RNA
tgcagtcgtgcacaaagttgt

SEQ ID NO: 29 - RNA, 21 nt, synthetic construct
  source 1..21: mol_type = other RNA
tttgacgcagtcgtgcacaaa

SEQ ID NO: 30 - RNA, 21 nt, synthetic construct
  source 1..21: mol_type = other RNA
tattgacgcagtcgtgcacaa

SEQ ID NO: 31 - RNA, 21 nt, synthetic construct
  source 1..21: mol_type = other RNA
ttattgacgcagtcgtgcaca

SEQ ID NO: 32 - RNA, 21 nt, synthetic construct
  source 1..21: mol_type = other RNA
tatattgacgcagtcgtgcac

SEQ ID NO: 33 - RNA, 21 nt, synthetic construct
  source 1..21: mol_type = other RNA
tgatattgacgcagtcgtgca

SEQ ID NO: 34 - RNA, 21 nt, synthetic construct
  source 1..21: mol_type = other RNA
ttgatattgacgcagtcgtgc

SEQ ID NO: 35 - RNA, 21 nt, synthetic construct
  source 1..21: mol_type = other RNA
tgcttgattgtgatattgacg

SEQ ID NO: 36 - RNA, 21 nt, synthetic construct
  source 1..21: mol_type = other RNA
tctgcttgattgtgatattga

SEQ ID NO: 37 - RNA, 21 nt, synthetic construct
  source 1..21: mol_type = other RNA
tgctgcttgattgtgatattg

SEQ ID NO: 38 - RNA, 21 nt, synthetic construct
  source 1..21: mol_type = other RNA
tgtgctgcttgattgtgatat

SEQ ID NO: 39 - RNA, 21 nt, synthetic construct
  source 1..21: mol_type = other RNA
tgtgaccgtgtgctgcttgat

SEQ ID NO: 40 - RNA, 21 nt, synthetic construct
  source 1..21: mol_type = other RNA
tggtgaccgtgtgctgcttga

SEQ ID NO: 41 - RNA, 21 nt, synthetic construct
  source 1..21: mol_type = other RNA
tgtggtgaccgtgtgctgctt

SEQ ID NO: 42 - RNA, 21 nt, synthetic construct
  source 1..21: mol_type = other RNA
tgttgtggtgaccgtgtgctg

SEQ ID NO: 43 - RNA, 21 nt, synthetic construct
  source 1..21: mol_type = other RNA
tccttggtggttgtggtgacc

SEQ ID NO: 44 - RNA, 21 nt, synthetic construct
  source 1..21: mol_type = other RNA
tggtctcggtgaagttctccc

SEQ ID NO: 45 - RNA, 21 nt, synthetic construct
  source 1..21: mol_type = other RNA
tcgcgctccatcatcttaacg

SEQ ID NO: 46 - RNA, 21 nt, synthetic construct
  source 1..21: mol_type = other RNA
tccacgcgctccatcatctta

SEQ ID NO: 47 - RNA, 21 nt, synthetic construct
  source 1..21: mol_type = other RNA
tctgctcaaccacgcgctcca

SEQ ID NO: 48 - RNA, 21 nt, synthetic construct
  source 1..21: mol_type = other RNA
tatgctcgatcctctctggta

SEQ ID NO: 49 - RNA, 21 nt, synthetic construct
  source 1..21: mol_type = other RNA
tagaagaggaccatgctcgat

SEQ ID NO: 50 - RNA, 21 nt, synthetic construct
  source 1..21: mol_type = other RNA
tggagaagaggaccatgctcg

SEQ ID NO: 51 - RNA, 21 nt, synthetic construct
  source 1..21: mol_type = other RNA
tggaggatcacaggtggagag

SEQ ID NO: 52 - RNA, 21 nt, synthetic construct
  source 1..21: mol_type = other RNA
tagagatcaggaggatcacag

SEQ ID NO: 53 - RNA, 21 nt, synthetic construct
  source 1..21: mol_type = other RNA
tggaaagagatcaggaggatc

SEQ ID NO: 54 - RNA, 21 nt, synthetic construct
  source 1..21: mol_type = other RNA
ttcaggaagatgaggaaagag

SEQ ID NO: 55 - RNA, 21 nt, synthetic construct
  source 1..21: mol_type = other RNA
tgaagaccttcctcatcccac

SEQ ID NO: 56 - RNA, 21 nt, synthetic construct
  source 1..21: mol_type = other RNA
taaagatggtgaaaacaggaa

SEQ ID NO: 57 - RNA, 21 nt, synthetic construct
  source 1..21: mol_type = other RNA
tgaaagatggtgaaaacagga

SEQ ID NO: 58 - RNA, 21 nt, synthetic construct
  source 1..21: mol_type = other RNA
ttagaaagatggtgaaaacag

SEQ ID NO: 59 - RNA, 21 nt, synthetic construct
  source 1..21: mol_type = other RNA
tagattagaaagatggtgaaa

SEQ ID NO: 60 - RNA, 21 nt, synthetic construct
  source 1..21: mol_type = other RNA
tcctatccgggacaaagagag

SEQ ID NO: 61 - RNA, 21 nt, synthetic construct
  source 1..21: mol_type = other RNA
tattagcctatccgggacaaa

SEQ ID NO: 62 - RNA, 21 nt, synthetic construct
  source 1..21: mol_type = other RNA
ttgattagcctatccgggaca

SEQ ID NO: 63 - RNA, 21 nt, synthetic construct
  source 1..21: mol_type = other RNA
tttgattagcctatccgggac

SEQ ID NO: 64 - RNA, 21 nt, synthetic construct
  source 1..21: mol_type = other RNA
ttatgttttccagtgcccatc

SEQ ID NO: 65 - RNA, 21 nt, synthetic construct
  source 1..21: mol_type = other RNA
tttactggcctggcattagca

SEQ ID NO: 66 - RNA, 21 nt, synthetic construct
  source 1..21: mol_type = other RNA
tcttttactggcctggcatta

SEQ ID NO: 67 - RNA, 21 nt, synthetic construct
  source 1..21: mol_type = other RNA
taccaatggttatttgctgtt

SEQ ID NO: 68 - RNA, 21 nt, synthetic construct
  source 1..21: mol_type = other RNA
tataagtccagattaaccaat

SEQ ID NO: 69 - RNA, 21 nt, synthetic construct
  source 1..21: mol_type = other RNA
ttgttttagcctcaacctgtt

SEQ ID NO: 70 - RNA, 21 nt, synthetic construct
  source 1..21: mol_type = other RNA
tagatttgttttagcctcaac

SEQ ID NO: 71 - RNA, 21 nt, synthetic construct
  source 1..21: mol_type = other RNA
tgagatttgttttagcctcaa

SEQ ID NO: 72 - RNA, 21 nt, synthetic construct
  source 1..21: mol_type = other RNA
ttgagatttgttttagcctca

SEQ ID NO: 73 - RNA, 21 nt, synthetic construct
  source 1..21: mol_type = other RNA
tctgagatttgttttagcctc

SEQ ID NO: 74 - RNA, 21 nt, synthetic construct
  source 1..21: mol_type = other RNA
ttctgagatttgttttagcct

SEQ ID NO: 75 - RNA, 21 nt, synthetic construct
  source 1..21: mol_type = other RNA
ttcagactgttctgagatttg

SEQ ID NO: 76 - RNA, 21 nt, synthetic construct
  source 1..21: mol_type = other RNA
taaggtatttcagactgttct

SEQ ID NO: 77 - RNA, 21 nt, synthetic construct
  source 1..21: mol_type = other RNA
ttgaaggagccagaggtatcc

SEQ ID NO: 78 - RNA, 21 nt, synthetic construct
  source 1..21: mol_type = other RNA
tctgaaggagccagaggtatc

SEQ ID NO: 79 - RNA, 21 nt, synthetic construct
  source 1..21: mol_type = other RNA
tgctgaaggagccagaggtat

SEQ ID NO: 80 - RNA, 21 nt, synthetic construct
  source 1..21: mol_type = other RNA
tagatagggcattagtatact

SEQ ID NO: 81 - RNA, 21 nt, synthetic construct
  source 1..21: mol_type = other RNA
taagatagggcattagtatac

SEQ ID NO: 82 - RNA, 21 nt, synthetic construct
  source 1..21: mol_type = other RNA
taaatctctactaagataggg

SEQ ID NO: 83 - RNA, 21 nt, synthetic construct
  source 1..21: mol_type = other RNA
tgaaatctctactaagatagg

SEQ ID NO: 84 - RNA, 21 nt, synthetic construct
  source 1..21: mol_type = other RNA
tatagctatgaaatctctact

SEQ ID NO: 85 - RNA, 21 nt, synthetic construct
  source 1..21: mol_type = other RNA
ttaaatagctatgaaatctct

SEQ ID NO: 86 - RNA, 21 nt, synthetic construct
  source 1..21: mol_type = other RNA
ttcgggttttcttaaaatgga

SEQ ID NO: 87 - RNA, 21 nt, synthetic construct
  source 1..21: mol_type = other RNA
tgtcgggttttcttaaaatgg

SEQ ID NO: 88 - RNA, 21 nt, synthetic construct
  source 1..21: mol_type = other RNA
ttggcctcctaacaaacctgg

SEQ ID NO: 89 - RNA, 21 nt, synthetic construct
  source 1..21: mol_type = other RNA
ttatcatgtggcctcctaaca

SEQ ID NO: 90 - RNA, 21 nt, synthetic construct
  source 1..21: mol_type = other RNA
tgtatcatgtggcctcctaac

SEQ ID NO: 91 - RNA, 21 nt, synthetic construct
  source 1..21: mol_type = other RNA
ttaagtatcatgtggcctcct

SEQ ID NO: 92 - RNA, 21 nt, synthetic construct
  source 1..21: mol_type = other RNA
tccaagagctaagaatctcta

SEQ ID NO: 93 - RNA, 21 nt, synthetic construct
  source 1..21: mol_type = other RNA
tgttctcggtacacacagagc

SEQ ID NO: 94 - RNA, 21 nt, synthetic construct
  source 1..21: mol_type = other RNA
tatactgtgaaaagtaaaaca

SEQ ID NO: 95 - RNA, 21 nt, synthetic construct
  source 1..21: mol_type = other RNA
tacaatatttactcttgttga

SEQ ID NO: 96 - RNA, 21 nt, synthetic construct
  source 1..21: mol_type = other RNA
taatatgtcctctagccagag

SEQ ID NO: 97 - RNA, 21 nt, synthetic construct
  source 1..21: mol_type = other RNA
tgaatatgtcctctagccaga

SEQ ID NO: 98 - RNA, 21 nt, synthetic construct
  source 1..21: mol_type = other RNA
tacagttatgttcactgtgaa

SEQ ID NO: 99 - RNA, 21 nt, synthetic construct
  source 1..21: mol_type = other RNA
ttacagttatgttcactgtga

SEQ ID NO: 100 - RNA, 21 nt, synthetic construct
  source 1..21: mol_type = other RNA
tagaagcctttcatatatgtt

SEQ ID NO: 101 - RNA, 21 nt, synthetic construct
  source 1..21: mol_type = other RNA
ttcaagtcccagaagcctttc

SEQ ID NO: 102 - RNA, 21 nt, synthetic construct
  source 1..21: mol_type = other RNA
tatttgatttcaagtcccaga

SEQ ID NO: 103 - RNA, 21 nt, synthetic construct
  source 1..21: mol_type = other RNA
taaacatctaaaatgggaggt

SEQ ID NO: 104 - RNA, 21 nt, synthetic construct
  source 1..21: mol_type = other RNA
tatatagggtcctttaaacat

SEQ ID NO: 105 - RNA, 21 nt, synthetic construct
  source 1..21: mol_type = other RNA
taagaaaggaatgccacatat

SEQ ID NO: 106 - RNA, 21 nt, synthetic construct
  source 1..21: mol_type = other RNA
tcctatagtttaaagaaagga

SEQ ID NO: 107 - RNA, 21 nt, synthetic construct
  source 1..21: mol_type = other RNA
tagctgccttaattacctata

SEQ ID NO: 108 - RNA, 21 nt, synthetic construct
  source 1..21: mol_type = other RNA
tcagctgccttaattacctat

SEQ ID NO: 109 - RNA, 21 nt, synthetic construct
  source 1..21: mol_type = other RNA
tttcagctgccttaattacct

SEQ ID NO: 110 - RNA, 21 nt, synthetic construct
  source 1..21: mol_type = other RNA
tacttttcagctgccttaatt

SEQ ID NO: 111 - RNA, 21 nt, synthetic construct
  source 1..21: mol_type = other RNA
ttacttttcagctgccttaat

SEQ ID NO: 112 - RNA, 21 nt, synthetic construct
  source 1..21: mol_type = other RNA
tttacttttcagctgccttaa

SEQ ID NO: 113 - RNA, 21 nt, synthetic construct
  source 1..21: mol_type = other RNA
ttttacttttcagctgcctta

SEQ ID NO: 114 - RNA, 21 nt, synthetic construct
  source 1..21: mol_type = other RNA
tatttacttttcagctgcctt

SEQ ID NO: 115 - RNA, 21 nt, synthetic construct
  source 1..21: mol_type = other RNA
tggcaatttacttttcagctg

SEQ ID NO: 116 - RNA, 21 nt, synthetic construct
  source 1..21: mol_type = other RNA
tctagaaggcaatttactttt

SEQ ID NO: 117 - RNA, 21 nt, synthetic construct
  source 1..21: mol_type = other RNA
ttcagtgtctagaaggcaatt

SEQ ID NO: 118 - RNA, 21 nt, synthetic construct
  source 1..21: mol_type = other RNA
tttgccttcagtgtctagaag

SEQ ID NO: 119 - RNA, 21 nt, synthetic construct
  source 1..21: mol_type = other RNA
tacaaaggagatttgccttca

SEQ ID NO: 120 - RNA, 21 nt, synthetic construct
  source 1..21: mol_type = other RNA
taatggacaaaggagatttgc

SEQ ID NO: 121 - RNA, 21 nt, synthetic construct
  source 1..21: mol_type = other RNA
ttttccaggtaaatggacaaa

SEQ ID NO: 122 - RNA, 21 nt, synthetic construct
  source 1..21: mol_type = other RNA
tggtttccaggtaaatggaca

SEQ ID NO: 123 - RNA, 21 nt, synthetic construct
  source 1..21: mol_type = other RNA
ttctggtttccaggtaaatgg

SEQ ID NO: 124 - RNA, 21 nt, synthetic construct
  source 1..21: mol_type = other RNA
tcaaaatcattctggtttcca

SEQ ID NO: 125 - RNA, 21 nt, synthetic construct
  source 1..21: mol_type = other RNA
tagctctcctgtatgtcaaaa

SEQ ID NO: 126 - RNA, 21 nt, synthetic construct
  source 1..21: mol_type = other RNA
tgctttcacaactgcagctct

SEQ ID NO: 127 - RNA, 21 nt, synthetic construct
  source 1..21: mol_type = other RNA
tgatggtgctttcacaactgc

SEQ ID NO: 128 - RNA, 21 nt, synthetic construct
  source 1..21: mol_type = other RNA
tcctctatgatgatggtgctt

SEQ ID NO: 129 - RNA, 21 nt, synthetic construct
  source 1..21: mol_type = other RNA
tcatcctctatgatgatggtg

SEQ ID NO: 130 - RNA, 21 nt, synthetic construct
  source 1..21: mol_type = other RNA
ttacatcatcctctatgatga

SEQ ID NO: 131 - RNA, 21 nt, synthetic construct
  source 1..21: mol_type = other RNA
tttctttgcacactgaccatt

SEQ ID NO: 132 - RNA, 21 nt, synthetic construct
  source 1..21: mol_type = other RNA
tcttttctttgcacactgacc

SEQ ID NO: 133 - RNA, 21 nt, synthetic construct
  source 1..21: mol_type = other RNA
tttcttttctttgcacactga

SEQ ID NO: 134 - RNA, 21 nt, synthetic construct
  source 1..21: mol_type = other RNA
tcagttcttttctttgcacac

SEQ ID NO: 135 - RNA, 21 nt, synthetic construct
  source 1..21: mol_type = other RNA
tgcagttcttttctttgcaca

SEQ ID NO: 136 - RNA, 21 nt, synthetic construct
  source 1..21: mol_type = other RNA
tagcagttcttttctttgcac

SEQ ID NO: 137 - RNA, 21 nt, synthetic construct
  source 1..21: mol_type = other RNA
taaataaagaaatgcaagcag

SEQ ID NO: 138 - RNA, 21 nt, synthetic construct
  source 1..21: mol_type = other RNA
tgaaataaagaaatgcaagca

SEQ ID NO: 139 - RNA, 21 nt, synthetic construct
  source 1..21: mol_type = other RNA
tagaaataaagaaatgcaagc

SEQ ID NO: 140 - RNA, 21 nt, synthetic construct
  source 1..21: mol_type = other RNA
tagaaataaagaaatgcaagc

SEQ ID NO: 141 - RNA, 21 nt, synthetic construct
  source 1..21: mol_type = other RNA
tacaattatgagacagaaata

SEQ ID NO: 142 - RNA, 21 nt, synthetic construct
  source 1..21: mol_type = other RNA
tttgacaattatgagacagaa

SEQ ID NO: 143 - RNA, 21 nt, synthetic construct
  source 1..21: mol_type = other RNA
tactatgaacttgacctaatt

SEQ ID NO: 144 - RNA, 21 nt, synthetic construct
  source 1..21: mol_type = other RNA
taactatgaacttgacctaat

SEQ ID NO: 145 - RNA, 21 nt, synthetic construct
  source 1..21: mol_type = other RNA
taaactatgaacttgacctaa

SEQ ID NO: 146 - RNA, 21 nt, synthetic construct
  source 1..21: mol_type = other RNA
tgaaactatgaacttgaccta

SEQ ID NO: 147 - RNA, 21 nt, synthetic construct
  source 1..21: mol_type = other RNA
tagaaactatgaacttgacct

SEQ ID NO: 148 - RNA, 21 nt, synthetic construct
  source 1..21: mol_type = other RNA
tcagaaactatgaacttgacc

SEQ ID NO: 149 - RNA, 21 nt, synthetic construct
  source 1..21: mol_type = other RNA
tcaattacagaaactatgaac

SEQ ID NO: 150 - RNA, 21 nt, synthetic construct
  source 1..21: mol_type = other RNA
taaagccaattacagaaacta

SEQ ID NO: 151 - RNA, 21 nt, synthetic construct
  source 1..21: mol_type = other RNA
ttcaaaagccaattacagaaa

SEQ ID NO: 152 - RNA, 21 nt, synthetic construct
  source 1..21: mol_type = other RNA
tgattcaaaagccaattacag

SEQ ID NO: 153 - RNA, 21 nt, synthetic construct
  source 1..21: mol_type = other RNA
ttgattcaaaagccaattaca

SEQ ID NO: 154 - RNA, 21 nt, synthetic construct
  source 1..21: mol_type = other RNA
tttctttgattcaaaagccaa

SEQ ID NO: 155 - RNA, 21 nt, synthetic construct
  source 1..21: mol_type = other RNA
tctattctttgattcaaaagc

SEQ ID NO: 156 - RNA, 21 nt, synthetic construct
  source 1..21: mol_type = other RNA
tcctattctttgattcaaaag

SEQ ID NO: 157 - RNA, 21 nt, synthetic construct
  source 1..21: mol_type = other RNA
tctccctattctttgattcaa

SEQ ID NO: 158 - RNA, 21 nt, synthetic construct
  source 1..21: mol_type = other RNA
ttagattgtctccctattctt

SEQ ID NO: 159 - RNA, 21 nt, synthetic construct
  source 1..21: mol_type = other RNA
ttctgtcatctccaacctaag

SEQ ID NO: 160 - RNA, 21 nt, synthetic construct
  source 1..21: mol_type = other RNA
tttctgtcatctccaacctaa

SEQ ID NO: 161 - RNA, 21 nt, synthetic construct
  source 1..21: mol_type = other RNA
ttttctgtcatctccaaccta

SEQ ID NO: 162 - RNA, 21 nt, synthetic construct
  source 1..21: mol_type = other RNA
tatatttctgtcatctccaac

SEQ ID NO: 163 - RNA, 21 nt, synthetic construct
  source 1..21: mol_type = other RNA
tttccacttcaaatcaatcat

SEQ ID NO: 164 - RNA, 21 nt, synthetic construct
  source 1..21: mol_type = other RNA
ttttccacttcaaatcaatca

SEQ ID NO: 165 - RNA, 21 nt, synthetic construct
  source 1..21: mol_type = other RNA
tatcaaacaattcagggaata

SEQ ID NO: 166 - RNA, 21 nt, synthetic construct
  source 1..21: mol_type = other RNA
tggtgacaatatcaaacaatt

SEQ ID NO: 167 - RNA, 21 nt, synthetic construct
  source 1..21: mol_type = other RNA
tgctaggtgacaatatcaaac

SEQ ID NO: 168 - RNA, 21 nt, synthetic construct
  source 1..21: mol_type = other RNA
tctgctaggtgacaatatcaa

SEQ ID NO: 169 - RNA, 21 nt, synthetic construct
  source 1..21: mol_type = other RNA
tatatctgctaggtgacaata

SEQ ID NO: 170 - RNA, 21 nt, synthetic construct
  source 1..21: mol_type = other RNA
ttacatatctgctaggtgaca

SEQ ID NO: 171 - RNA, 21 nt, synthetic construct
  source 1..21: mol_type = other RNA
taaaagtaatacatatctgct

SEQ ID NO: 172 - RNA, 21 nt, synthetic construct
  source 1..21: mol_type = other RNA
tcaataataacattgcagaaa

SEQ ID NO: 173 - RNA, 21 nt, synthetic construct
  source 1..21: mol_type = other RNA
tccaataataacattgcagaa

SEQ ID NO: 174 - RNA, 21 nt, synthetic construct
  source 1..21: mol_type = other RNA
tgcaagccaataataacattg

SEQ ID NO: 175 - RNA, 21 nt, synthetic construct
  source 1..21: mol_type = other RNA
tcaaagtgcaagccaataata

SEQ ID NO: 176 - RNA, 21 nt, synthetic construct
  source 1..21: mol_type = other RNA
tcacaaagtgcaagccaataa

SEQ ID NO: 177 - RNA, 21 nt, synthetic construct
  source 1..21: mol_type = other RNA
taatactcacaaagtgcaagc

SEQ ID NO: 178 - RNA, 21 nt, synthetic construct
  source 1..21: mol_type = other RNA
tgaatactcacaaagtgcaag

SEQ ID NO: 179 - RNA, 21 nt, synthetic construct
  source 1..21: mol_type = other RNA
tagaatactcacaaagtgcaa

SEQ ID NO: 180 - RNA, 21 nt, synthetic construct
  source 1..21: mol_type = other RNA
ttagaatactcacaaagtgca

SEQ ID NO: 181 - RNA, 21 nt, synthetic construct
  source 1..21: mol_type = other RNA
ttagaatactcacaaagtgca

SEQ ID NO: 182 - RNA, 21 nt, synthetic construct
  source 1..21: mol_type = other RNA
tatagaatactcacaaagtgc

SEQ ID NO: 183 - RNA, 21 nt, synthetic construct
  source 1..21: mol_type = other RNA
tacatagaatactcacaaagt

SEQ ID NO: 184 - RNA, 21 nt, synthetic construct
  source 1..21: mol_type = other RNA
tgtctgtcctatgcaatatat

SEQ ID NO: 185 - RNA, 21 nt, synthetic construct
  source 1..21: mol_type = other RNA
taagtctgtcctatgcaatat

SEQ ID NO: 186 - RNA, 21 nt, synthetic construct
  source 1..21: mol_type = other RNA
ttaagtctgtcctatgcaata

SEQ ID NO: 187 - RNA, 21 nt, synthetic construct
  source 1..21: mol_type = other RNA
tctaagtctgtcctatgcaat

SEQ ID NO: 188 - RNA, 21 nt, synthetic construct
  source 1..21: mol_type = other RNA
ttcctaagtctgtcctatgca

SEQ ID NO: 189 - RNA, 21 nt, synthetic construct
  source 1..21: mol_type = other RNA
taaactcctaagtctgtccta

SEQ ID NO: 190 - RNA, 21 nt, synthetic construct
  source 1..21: mol_type = other RNA
tactgctctaaacaaaactcc

SEQ ID NO: 191 - RNA, 21 nt, synthetic construct
  source 1..21: mol_type = other RNA
tagatgttaactgctctaaac

SEQ ID NO: 192 - RNA, 21 nt, synthetic construct
  source 1..21: mol_type = other RNA
tcagatgttaactgctctaaa

SEQ ID NO: 193 - RNA, 21 nt, synthetic construct
  source 1..21: mol_type = other RNA
ttcagatgttaactgctctaa

SEQ ID NO: 194 - RNA, 21 nt, synthetic construct
  source 1..21: mol_type = other RNA
tttcagatgttaactgctcta

SEQ ID NO: 195 - RNA, 21 nt, synthetic construct
  source 1..21: mol_type = other RNA
tcttcagatgttaactgctct

SEQ ID NO: 196 - RNA, 21 nt, synthetic construct
  source 1..21: mol_type = other RNA
tttagacacttcagatgttaa

SEQ ID NO: 197 - RNA, 21 nt, synthetic construct
  source 1..21: mol_type = other RNA
tgcattagacacttcagatgt

SEQ ID NO: 198 - RNA, 21 nt, synthetic construct
  source 1..21: mol_type = other RNA
taaaagttaatgcattagaca

SEQ ID NO: 199 - RNA, 21 nt, synthetic construct
  source 1..21: mol_type = other RNA
tagtaccttacaaaagttaat

SEQ ID NO: 200 - RNA, 21 nt, synthetic construct
  source 1..21: mol_type = other RNA
tcagtaccttacaaaagttaa

SEQ ID NO: 201 - RNA, 21 nt, synthetic construct
  source 1..21: mol_type = other RNA
ttcagtaccttacaaaagtta

SEQ ID NO: 202 - RNA, 21 nt, synthetic construct
  source 1..21: mol_type = other RNA
tttcagtaccttacaaaagtt

SEQ ID NO: 203 - RNA, 21 nt, synthetic construct
  source 1..21: mol_type = other RNA
tattcagtaccttacaaaagt

SEQ ID NO: 204 - RNA, 21 nt, synthetic construct
  source 1..21: mol_type = other RNA
ttaagtattcagtaccttaca

SEQ ID NO: 205 - RNA, 21 nt, synthetic construct
  source 1..21: mol_type = other RNA
ttcccacatattaagtattca

SEQ ID NO: 206 - RNA, 21 nt, synthetic construct
  source 1..21: mol_type = other RNA
tttgtaagcctaaggaccacg

SEQ ID NO: 207 - RNA, 21 nt, synthetic construct
  source 1..21: mol_type = other RNA
tcatgaaacgattcagtgcac

SEQ ID NO: 208 - RNA, 21 nt, synthetic construct
  source 1..21: mol_type = other RNA
tattcttacatgaaacgattc

SEQ ID NO: 209 - RNA, 21 nt, synthetic construct
  source 1..21: mol_type = other RNA
tttggattcttacatgaaacg

SEQ ID NO: 210 - RNA, 21 nt, synthetic construct
  source 1..21: mol_type = other RNA
tcactttggattcttacatga

SEQ ID NO: 211 - RNA, 21 nt, synthetic construct
  source 1..21: mol_type = other RNA
taatggtgtccactttggatt

SEQ ID NO: 212 - RNA, 21 nt, synthetic construct
  source 1..21: mol_type = other RNA
tttaatggtgtccactttgga

SEQ ID NO: 213 - RNA, 21 nt, synthetic construct
  source 1..21: mol_type = other RNA
taaagacctgttaatggtgtc

SEQ ID NO: 214 - RNA, 21 nt, synthetic construct
  source 1..21: mol_type = other RNA
ttgcatatttcaaagacctgt

SEQ ID NO: 215 - RNA, 21 nt, synthetic construct
  source 1..21: mol_type = other RNA
taacatgcaaagttacaaata

SEQ ID NO: 216 - RNA, 21 nt, synthetic construct
  source 1..21: mol_type = other RNA
tgaacatgcaaagttacaaat

SEQ ID NO: 217 - RNA, 21 nt, synthetic construct
  source 1..21: mol_type = other RNA
tagaacatgcaaagttacaaa

SEQ ID NO: 218 - RNA, 21 nt, synthetic construct
  source 1..21: mol_type = other RNA
taagaacatgcaaagttacaa

SEQ ID NO: 219 - RNA, 21 nt, synthetic construct
  source 1..21: mol_type = other RNA
tcaagaacatgcaaagttaca

SEQ ID NO: 220 - RNA, 21 nt, synthetic construct
  source 1..21: mol_type = other RNA
tcaagaacatgcaaagttaca

SEQ ID NO: 221 - RNA, 21 nt, synthetic construct
  source 1..21: mol_type = other RNA
tacaagaacatgcaaagttac

SEQ ID NO: 222 - RNA, 21 nt, synthetic construct
  source 1..21: mol_type = other RNA
taacaagaacatgcaaagtta

SEQ ID NO: 223 - RNA, 21 nt, synthetic construct
  source 1..21: mol_type = other RNA
taaacaagaacatgcaaagtt

SEQ ID NO: 224 - RNA, 21 nt, synthetic construct
  source 1..21: mol_type = other RNA
taaaacaagaacatgcaaagt

SEQ ID NO: 225 - RNA, 21 nt, synthetic construct
  source 1..21: mol_type = other RNA
tcaaaacaagaacatgcaaag

SEQ ID NO: 226 - RNA, 21 nt, synthetic construct
  source 1..21: mol_type = other RNA
tcaaaacaagaacatgcaaag

SEQ ID NO: 227 - RNA, 21 nt, synthetic construct
  source 1..21: mol_type = other RNA
tacaaaacaagaacatgcaaa

SEQ ID NO: 228 - RNA, 21 nt, synthetic construct
  source 1..21: mol_type = other RNA
tgtttaatttcagtcagatat

SEQ ID NO: 229 - RNA, 21 nt, synthetic construct
  source 1..21: mol_type = other RNA
tgctcgtttaatttcagtcag

SEQ ID NO: 230 - RNA, 21 nt, synthetic construct
  source 1..21: mol_type = other RNA
tcgctcgtttaatttcagtca

SEQ ID NO: 231 - RNA, 21 nt, synthetic construct
  source 1..21: mol_type = other RNA
ttcgctcgtttaatttcagtc

SEQ ID NO: 232 - RNA, 21 nt, synthetic construct
  source 1..21: mol_type = other RNA
tttcgctcgtttaatttcagt

SEQ ID NO: 233 - RNA, 21 nt, synthetic construct
  source 1..21: mol_type = other RNA
tcttcgctcgtttaatttcag

SEQ ID NO: 234 - RNA, 21 nt, synthetic construct
  source 1..21: mol_type = other RNA
tacggtagtagcggtcctccc

SEQ ID NO: 235 - RNA, 21 nt, synthetic construct
  source 1..21: mol_type = other RNA
tgtggtgaccgtgtgctgctt

SEQ ID NO: 236 - RNA, 21 nt, synthetic construct
  source 1..21: mol_type = other RNA
tggagatgaggaggatgacag

SEQ ID NO: 237 - RNA, 21 nt, synthetic construct
  source 1..21: mol_type = other RNA
tttattcactagaccaaaaca

SEQ ID NO: 238 - RNA, 21 nt, synthetic construct
  source 1..21: mol_type = other RNA
tgtatttattcactagaccaa

SEQ ID NO: 239 - RNA, 21 nt, synthetic construct
  source 1..21: mol_type = other RNA
tgctctagctatcccaggtgg

SEQ ID NO: 240 - RNA, 21 nt, synthetic construct
  source 1..21: mol_type = other RNA
tttgctttcaacgtcagtagg

SEQ ID NO: 241 - RNA, 21 nt, synthetic construct
  source 1..21: mol_type = other RNA
ttctagtgccctgggaatgaa

SEQ ID NO: 242 - RNA, 21 nt, synthetic construct
  source 1..21: mol_type = other RNA
tagcttttgataattgtgaga

SEQ ID NO: 243 - RNA, 21 nt, synthetic construct
  source 1..21: mol_type = other RNA
tagtacagaaacatagggcag

SEQ ID NO: 244 - RNA, 21 nt, synthetic construct
  source 1..21: mol_type = other RNA
tagcagaatttctttcccact

SEQ ID NO: 245 - RNA, 21 nt, synthetic construct
  source 1..21: mol_type = other RNA
taaagaagacataagcttgaa

SEQ ID NO: 246 - RNA, 21 nt, synthetic construct
  source 1..21: mol_type = other RNA
taaaatacatgcaaagttaca

SEQ ID NO: 247 - RNA, 16 nt, synthetic construct
  source 1..16: mol_type = other RNA
tcaatgagccacgtca

SEQ ID NO: 248 - RNA, 16 nt, synthetic construct
  source 1..16: mol_type = other RNA
ctgatgcaagtgttca

SEQ ID NO: 249 - RNA, 16 nt, synthetic construct
  source 1..16: mol_type = other RNA
ctgcaccctttaacta

SEQ ID NO: 250 - RNA, 16 nt, synthetic construct
  source 1..16: mol_type = other RNA
ctccgagccagtcgca

SEQ ID NO: 251 - RNA, 16 nt, synthetic construct
  source 1..16: mol_type = other RNA
agcttctcctctccta

SEQ ID NO: 252 - RNA, 16 nt, synthetic construct
  source 1..16: mol_type = other RNA
cttctcctctcctcaa

SEQ ID NO: 253 - RNA, 16 nt, synthetic construct
  source 1..16: mol_type = other RNA
tcctctcctcacgaca

SEQ ID NO: 254 - RNA, 16 nt, synthetic construct
  source 1..16: mol_type = other RNA
ggtggcacccacagta

SEQ ID NO: 255 - RNA, 16 nt, synthetic construct
  source 1..16: mol_type = other RNA
aagccgagtaagccaa

SEQ ID NO: 256 - RNA, 16 nt, synthetic construct
  source 1..16: mol_type = other RNA
ggctacatgctgggaa

SEQ ID NO: 257 - RNA, 16 nt, synthetic construct
  source 1..16: mol_type = other RNA
ccatcatacatttcga

SEQ ID NO: 258 - RNA, 16 nt, synthetic construct
  source 1..16: mol_type = other RNA
ttcggcagtgactata

SEQ ID NO: 259 - RNA, 16 nt, synthetic construct
  source 1..16: mol_type = other RNA
gcagtgactatgagga

SEQ ID NO: 260 - RNA, 16 nt, synthetic construct
  source 1..16: mol_type = other RNA
agtgactatgaggaca

SEQ ID NO: 261 - RNA, 16 nt, synthetic construct
  source 1..16: mol_type = other RNA
actatgaggaccgtta

SEQ ID NO: 262 - RNA, 16 nt, synthetic construct
  source 1..16: mol_type = other RNA
gaccgttactatcgta

SEQ ID NO: 263 - RNA, 16 nt, synthetic construct
  source 1..16: mol_type = other RNA
cgtgaaaacatgcaca

SEQ ID NO: 264 - RNA, 16 nt, synthetic construct
  source 1..16: mol_type = other RNA
catgcaccgttaccca

SEQ ID NO: 265 - RNA, 16 nt, synthetic construct
  source 1..16: mol_type = other RNA
caagtgtactacagga

SEQ ID NO: 266 - RNA, 16 nt, synthetic construct
  source 1..16: mol_type = other RNA
aagtgtactacaggca

SEQ ID NO: 267 - RNA, 16 nt, synthetic construct
  source 1..16: mol_type = other RNA
tacaggcccatggata

SEQ ID NO: 268 - RNA, 16 nt, synthetic construct
  source 1..16: mol_type = other RNA
aggcccatggatgaga

SEQ ID NO: 269 - RNA, 16 nt, synthetic construct
  source 1..16: mol_type = other RNA
ggcccatggatgagta

SEQ ID NO: 270 - RNA, 16 nt, synthetic construct
  source 1..16: mol_type = other RNA
atggatgagtacagca

SEQ ID NO: 271 - RNA, 16 nt, synthetic construct
  source 1..16: mol_type = other RNA
gagtacagcaaccaga

SEQ ID NO: 272 - RNA, 16 nt, synthetic construct
  source 1..16: mol_type = other RNA
tacagcaaccagaaca

SEQ ID NO: 273 - RNA, 16 nt, synthetic construct
  source 1..16: mol_type = other RNA
agcaaccagaacaaca

SEQ ID NO: 274 - RNA, 16 nt, synthetic construct
  source 1..16: mol_type = other RNA
tttgtgcacgactgca

SEQ ID NO: 275 - RNA, 16 nt, synthetic construct
  source 1..16: mol_type = other RNA
gcacgactgcgtcaaa

SEQ ID NO: 276 - RNA, 16 nt, synthetic construct
  source 1..16: mol_type = other RNA
cacgactgcgtcaata

SEQ ID NO: 277 - RNA, 16 nt, synthetic construct
  source 1..16: mol_type = other RNA
acgactgcgtcaataa

SEQ ID NO: 278 - RNA, 16 nt, synthetic construct
  source 1..16: mol_type = other RNA
cgactgcgtcaatata

SEQ ID NO: 279 - RNA, 16 nt, synthetic construct
  source 1..16: mol_type = other RNA
gactgcgtcaatatca

SEQ ID NO: 280 - RNA, 16 nt, synthetic construct
  source 1..16: mol_type = other RNA
actgcgtcaatatcaa

SEQ ID NO: 281 - RNA, 16 nt, synthetic construct
  source 1..16: mol_type = other RNA
atatcacaatcaagca

SEQ ID NO: 282 - RNA, 16 nt, synthetic construct
  source 1..16: mol_type = other RNA
atcacaatcaagcaga

SEQ ID NO: 283 - RNA, 16 nt, synthetic construct
  source 1..16: mol_type = other RNA
tcacaatcaagcagca

SEQ ID NO: 284 - RNA, 16 nt, synthetic construct
  source 1..16: mol_type = other RNA
acaatcaagcagcaca

SEQ ID NO: 285 - RNA, 16 nt, synthetic construct
  source 1..16: mol_type = other RNA
gcagcacacggtcaca

SEQ ID NO: 286 - RNA, 16 nt, synthetic construct
  source 1..16: mol_type = other RNA
cagcacacggtcacca

SEQ ID NO: 287 - RNA, 16 nt, synthetic construct
  source 1..16: mol_type = other RNA
gcacacggtcaccaca

SEQ ID NO: 288 - RNA, 16 nt, synthetic construct
  source 1..16: mol_type = other RNA
cacggtcaccacaaca

SEQ ID NO: 289 - RNA, 16 nt, synthetic construct
  source 1..16: mol_type = other RNA
ccacaaccaccaagga

SEQ ID NO: 290 - RNA, 16 nt, synthetic construct
  source 1..16: mol_type = other RNA
aacttcaccgagacca

SEQ ID NO: 291 - RNA, 16 nt, synthetic construct
  source 1..16: mol_type = other RNA
agatgatggagcgcga

SEQ ID NO: 292 - RNA, 16 nt, synthetic construct
  source 1..16: mol_type = other RNA
tgatggagcgcgtgga

SEQ ID NO: 293 - RNA, 16 nt, synthetic construct
  source 1..16: mol_type = other RNA
cgcgtggttgagcaga

SEQ ID NO: 294 - RNA, 16 nt, synthetic construct
  source 1..16: mol_type = other RNA
gagaggatcgagcata

SEQ ID NO: 295 - RNA, 16 nt, synthetic construct
  source 1..16: mol_type = other RNA
gcatggtcctcttcta

SEQ ID NO: 296 - RNA, 16 nt, synthetic construct
  source 1..16: mol_type = other RNA
atggtcctcttctcca

SEQ ID NO: 297 - RNA, 16 nt, synthetic construct
  source 1..16: mol_type = other RNA
cacctgtgatcctcca

SEQ ID NO: 298 - RNA, 16 nt, synthetic construct
  source 1..16: mol_type = other RNA
atcctcctgatctcta

SEQ ID NO: 299 - RNA, 16 nt, synthetic construct
  source 1..16: mol_type = other RNA
tcctgatctctttcca

SEQ ID NO: 300 - RNA, 16 nt, synthetic construct
  source 1..16: mol_type = other RNA
tcctcatcttcctgaa

SEQ ID NO: 301 - RNA, 16 nt, synthetic construct
  source 1..16: mol_type = other RNA
atgaggaaggtcttca

SEQ ID NO: 302 - RNA, 16 nt, synthetic construct
  source 1..16: mol_type = other RNA
gttttcaccatcttta

SEQ ID NO: 303 - RNA, 16 nt, synthetic construct
  source 1..16: mol_type = other RNA
ttttcaccatctttca

SEQ ID NO: 304 - RNA, 16 nt, synthetic construct
  source 1..16: mol_type = other RNA
ttcaccatctttctaa

SEQ ID NO: 305 - RNA, 16 nt, synthetic construct
  source 1..16: mol_type = other RNA
ccatctttctaatcta

SEQ ID NO: 306 - RNA, 16 nt, synthetic construct
  source 1..16: mol_type = other RNA
tttgtcccggatagga

SEQ ID NO: 307 - RNA, 16 nt, synthetic construct
  source 1..16: mol_type = other RNA
cccggataggctaata

SEQ ID NO: 308 - RNA, 16 nt, synthetic construct
  source 1..16: mol_type = other RNA
cggataggctaatcaa

SEQ ID NO: 309 - RNA, 16 nt, synthetic construct
  source 1..16: mol_type = other RNA
ggataggctaatcaaa

SEQ ID NO: 310 - RNA, 16 nt, synthetic construct
  source 1..16: mol_type = other RNA
gcactggaaaacataa

SEQ ID NO: 311 - RNA, 16 nt, synthetic construct
  source 1..16: mol_type = other RNA
atgccaggccagtaaa

SEQ ID NO: 312 - RNA, 16 nt, synthetic construct
  source 1..16: mol_type = other RNA
ccaggccagtaaaaga

SEQ ID NO: 313 - RNA, 16 nt, synthetic construct
  source 1..16: mol_type = other RNA
caaataaccattggta

SEQ ID NO: 314 - RNA, 16 nt, synthetic construct
  source 1..16: mol_type = other RNA
ttaatctggacttata

SEQ ID NO: 315 - RNA, 16 nt, synthetic construct
  source 1..16: mol_type = other RNA
gttgaggctaaaacaa

SEQ ID NO: 316 - RNA, 16 nt, synthetic construct
  source 1..16: mol_type = other RNA
ggctaaaacaaatcta

SEQ ID NO: 317 - RNA, 16 nt, synthetic construct
  source 1..16: mol_type = other RNA
gctaaaacaaatctca

SEQ ID NO: 318 - RNA, 16 nt, synthetic construct
  source 1..16: mol_type = other RNA
ctaaaacaaatctcaa

SEQ ID NO: 319 - RNA, 16 nt, synthetic construct
  source 1..16: mol_type = other RNA
taaaacaaatctcaga

SEQ ID NO: 320 - RNA, 16 nt, synthetic construct
  source 1..16: mol_type = other RNA
aaaacaaatctcagaa

SEQ ID NO: 321 - RNA, 16 nt, synthetic construct
  source 1..16: mol_type = other RNA
ctcagaacagtctgaa

SEQ ID NO: 322 - RNA, 16 nt, synthetic construct
  source 1..16: mol_type = other RNA
agtctgaaatacctta

SEQ ID NO: 323 - RNA, 16 nt, synthetic construct
  source 1..16: mol_type = other RNA
cctctggctccttcaa

SEQ ID NO: 324 - RNA, 16 nt, synthetic construct
  source 1..16: mol_type = other RNA
ctctggctccttcaga

SEQ ID NO: 325 - RNA, 16 nt, synthetic construct
  source 1..16: mol_type = other RNA
tctggctccttcagca

SEQ ID NO: 326 - RNA, 16 nt, synthetic construct
  source 1..16: mol_type = other RNA
actaatgccctatcta

SEQ ID NO: 327 - RNA, 16 nt, synthetic construct
  source 1..16: mol_type = other RNA
ctaatgccctatctta

SEQ ID NO: 328 - RNA, 16 nt, synthetic construct
  source 1..16: mol_type = other RNA
tcttagtagagattta

SEQ ID NO: 329 - RNA, 16 nt, synthetic construct
  source 1..16: mol_type = other RNA
cttagtagagatttca

SEQ ID NO: 330 - RNA, 16 nt, synthetic construct
  source 1..16: mol_type = other RNA
agatttcatagctata

SEQ ID NO: 331 - RNA, 16 nt, synthetic construct
  source 1..16: mol_type = other RNA
tttcatagctatttaa

SEQ ID NO: 332 - RNA, 16 nt, synthetic construct
  source 1..16: mol_type = other RNA
tttaagaaaacccgaa

SEQ ID NO: 333 - RNA, 16 nt, synthetic construct
  source 1..16: mol_type = other RNA
ttaagaaaacccgaca

SEQ ID NO: 334 - RNA, 16 nt, synthetic construct
  source 1..16: mol_type = other RNA
tttgttaggaggccaa

SEQ ID NO: 335 - RNA, 16 nt, synthetic construct
  source 1..16: mol_type = other RNA
ggaggccacatgataa

SEQ ID NO: 336 - RNA, 16 nt, synthetic construct
  source 1..16: mol_type = other RNA
gaggccacatgataca

SEQ ID NO: 337 - RNA, 16 nt, synthetic construct
  source 1..16: mol_type = other RNA
gccacatgatacttaa

SEQ ID NO: 338 - RNA, 16 nt, synthetic construct
  source 1..16: mol_type = other RNA
attcttagctcttgga

SEQ ID NO: 339 - RNA, 16 nt, synthetic construct
  source 1..16: mol_type = other RNA
gtgtgtaccgagaaca

SEQ ID NO: 340 - RNA, 16 nt, synthetic construct
  source 1..16: mol_type = other RNA
tacttttcacagtata

SEQ ID NO: 341 - RNA, 16 nt, synthetic construct
  source 1..16: mol_type = other RNA
aagagtaaatattgta

SEQ ID NO: 342 - RNA, 16 nt, synthetic construct
  source 1..16: mol_type = other RNA
gctagaggacatatta

SEQ ID NO: 343 - RNA, 16 nt, synthetic construct
  source 1..16: mol_type = other RNA
ctagaggacatattca

SEQ ID NO: 344 - RNA, 16 nt, synthetic construct
  source 1..16: mol_type = other RNA
agtgaacataactgta

SEQ ID NO: 345 - RNA, 16 nt, synthetic construct
  source 1..16: mol_type = other RNA
gtgaacataactgtaa

SEQ ID NO: 346 - RNA, 16 nt, synthetic construct
  source 1..16: mol_type = other RNA
atatgaaaggcttcta

SEQ ID NO: 347 - RNA, 16 nt, synthetic construct
  source 1..16: mol_type = other RNA
gcttctgggacttgaa

SEQ ID NO: 348 - RNA, 16 nt, synthetic construct
  source 1..16: mol_type = other RNA
gacttgaaatcaaata

SEQ ID NO: 349 - RNA, 16 nt, synthetic construct
  source 1..16: mol_type = other RNA
ccattttagatgttta

SEQ ID NO: 350 - RNA, 16 nt, synthetic construct
  source 1..16: mol_type = other RNA
taaaggaccctatata

SEQ ID NO: 351 - RNA, 16 nt, synthetic construct
  source 1..16: mol_type = other RNA
tggcattcctttctta

SEQ ID NO: 352 - RNA, 16 nt, synthetic construct
  source 1..16: mol_type = other RNA
tctttaaactatagga

SEQ ID NO: 353 - RNA, 16 nt, synthetic construct
  source 1..16: mol_type = other RNA
gtaattaaggcagcta

SEQ ID NO: 354 - RNA, 16 nt, synthetic construct
  source 1..16: mol_type = other RNA
taattaaggcagctga

SEQ ID NO: 355 - RNA, 16 nt, synthetic construct
  source 1..16: mol_type = other RNA
attaaggcagctgaaa

SEQ ID NO: 356 - RNA, 16 nt, synthetic construct
  source 1..16: mol_type = other RNA
aggcagctgaaaagta

SEQ ID NO: 357 - RNA, 16 nt, synthetic construct
  source 1..16: mol_type = other RNA
ggcagctgaaaagtaa

SEQ ID NO: 358 - RNA, 16 nt, synthetic construct
  source 1..16: mol_type = other RNA
gcagctgaaaagtaaa

SEQ ID NO: 359 - RNA, 16 nt, synthetic construct
  source 1..16: mol_type = other RNA
cagctgaaaagtaaaa

SEQ ID NO: 360 - RNA, 16 nt, synthetic construct
  source 1..16: mol_type = other RNA
agctgaaaagtaaata

SEQ ID NO: 361 - RNA, 16 nt, synthetic construct
  source 1..16: mol_type = other RNA
gaaaagtaaattgcca

SEQ ID NO: 362 - RNA, 16 nt, synthetic construct
  source 1..16: mol_type = other RNA
taaattgccttctaga

SEQ ID NO: 363 - RNA, 16 nt, synthetic construct
  source 1..16: mol_type = other RNA
ccttctagacactgaa

SEQ ID NO: 364 - RNA, 16 nt, synthetic construct
  source 1..16: mol_type = other RNA
agacactgaaggcaaa

SEQ ID NO: 365 - RNA, 16 nt, synthetic construct
  source 1..16: mol_type = other RNA
gcaaatctcctttgta

SEQ ID NO: 366 - RNA, 16 nt, synthetic construct
  source 1..16: mol_type = other RNA
tctcctttgtccatta

SEQ ID NO: 367 - RNA, 16 nt, synthetic construct
  source 1..16: mol_type = other RNA
ccatttacctggaaaa

SEQ ID NO: 368 - RNA, 16 nt, synthetic construct
  source 1..16: mol_type = other RNA
atttacctggaaacca

SEQ ID NO: 369 - RNA, 16 nt, synthetic construct
  source 1..16: mol_type = other RNA
tacctggaaaccagaa

SEQ ID NO: 370 - RNA, 16 nt, synthetic construct
  source 1..16: mol_type = other RNA
accagaatgattttga

SEQ ID NO: 371 - RNA, 16 nt, synthetic construct
  source 1..16: mol_type = other RNA
acatacaggagagcta

SEQ ID NO: 372 - RNA, 16 nt, synthetic construct
  source 1..16: mol_type = other RNA
tgcagttgtgaaagca

SEQ ID NO: 373 - RNA, 16 nt, synthetic construct
  source 1..16: mol_type = other RNA
tgtgaaagcaccatca

SEQ ID NO: 374 - RNA, 16 nt, synthetic construct
  source 1..16: mol_type = other RNA
ccatcatcatagagga

SEQ ID NO: 375 - RNA, 16 nt, synthetic construct
  source 1..16: mol_type = other RNA
tcatcatagaggatga

SEQ ID NO: 376 - RNA, 16 nt, synthetic construct
  source 1..16: mol_type = other RNA
atagaggatgatgtaa

SEQ ID NO: 377 - RNA, 16 nt, synthetic construct
  source 1..16: mol_type = other RNA
tcagtgtgcaaagaaa

SEQ ID NO: 378 - RNA, 16 nt, synthetic construct
  source 1..16: mol_type = other RNA
gtgtgcaaagaaaaga

SEQ ID NO: 379 - RNA, 16 nt, synthetic construct
  source 1..16: mol_type = other RNA
gtgcaaagaaaagaaa

SEQ ID NO: 380 - RNA, 16 nt, synthetic construct
  source 1..16: mol_type = other RNA
caaagaaaagaactga

SEQ ID NO: 381 - RNA, 16 nt, synthetic construct
  source 1..16: mol_type = other RNA
aaagaaaagaactgca

SEQ ID NO: 382 - RNA, 16 nt, synthetic construct
  source 1..16: mol_type = other RNA
aagaaaagaactgcta

SEQ ID NO: 383 - RNA, 16 nt, synthetic construct
  source 1..16: mol_type = other RNA
tgcatttctttattta

SEQ ID NO: 384 - RNA, 16 nt, synthetic construct
  source 1..16: mol_type = other RNA
gcatttctttatttca

SEQ ID NO: 385 - RNA, 16 nt, synthetic construct
  source 1..16: mol_type = other RNA
catttctttatttcta

SEQ ID NO: 386 - RNA, 16 nt, synthetic construct
  source 1..16: mol_type = other RNA
catttctttatttcta

SEQ ID NO: 387 - RNA, 16 nt, synthetic construct
  source 1..16: mol_type = other RNA
ctgtctcataattgta

SEQ ID NO: 388 - RNA, 16 nt, synthetic construct
  source 1..16: mol_type = other RNA
tctcataattgtcaaa

SEQ ID NO: 389 - RNA, 16 nt, synthetic construct
  source 1..16: mol_type = other RNA
ggtcaagttcatagta

SEQ ID NO: 390 - RNA, 16 nt, synthetic construct
  source 1..16: mol_type = other RNA
gtcaagttcatagtta

SEQ ID NO: 391 - RNA, 16 nt, synthetic construct
  source 1..16: mol_type = other RNA
tcaagttcatagttta

SEQ ID NO: 392 - RNA, 16 nt, synthetic construct
  source 1..16: mol_type = other RNA
caagttcatagtttca

SEQ ID NO: 393 - RNA, 16 nt, synthetic construct
  source 1..16: mol_type = other RNA
aagttcatagtttcta

SEQ ID NO: 394 - RNA, 16 nt, synthetic construct
  source 1..16: mol_type = other RNA
agttcatagtttctga

SEQ ID NO: 395 - RNA, 16 nt, synthetic construct
  source 1..16: mol_type = other RNA
tagtttctgtaattga

SEQ ID NO: 396 - RNA, 16 nt, synthetic construct
  source 1..16: mol_type = other RNA
tctgtaattggcttta

SEQ ID NO: 397 - RNA, 16 nt, synthetic construct
  source 1..16: mol_type = other RNA
gtaattggcttttgaa

SEQ ID NO: 398 - RNA, 16 nt, synthetic construct
  source 1..16: mol_type = other RNA
attggcttttgaatca

SEQ ID NO: 399 - RNA, 16 nt, synthetic construct
  source 1..16: mol_type = other RNA
ttggcttttgaatcaa

SEQ ID NO: 400 - RNA, 16 nt, synthetic construct
  source 1..16: mol_type = other RNA
ttttgaatcaaagaaa

SEQ ID NO: 401 - RNA, 16 nt, synthetic construct
  source 1..16: mol_type = other RNA
tgaatcaaagaataga

SEQ ID NO: 402 - RNA, 16 nt, synthetic construct
  source 1..16: mol_type = other RNA
gaatcaaagaatagga

SEQ ID NO: 403 - RNA, 16 nt, synthetic construct
  source 1..16: mol_type = other RNA
tcaaagaatagggaga

SEQ ID NO: 404 - RNA, 16 nt, synthetic construct
  source 1..16: mol_type = other RNA
tagggagacaatctaa

SEQ ID NO: 405 - RNA, 16 nt, synthetic construct
  source 1..16: mol_type = other RNA
gttggagatgacagaa

SEQ ID NO: 406 - RNA, 16 nt, synthetic construct
  source 1..16: mol_type = other RNA
ttggagatgacagaaa

SEQ ID NO: 407 - RNA, 16 nt, synthetic construct
  source 1..16: mol_type = other RNA
tggagatgacagaaaa

SEQ ID NO: 408 - RNA, 16 nt, synthetic construct
  source 1..16: mol_type = other RNA
agatgacagaaatata

SEQ ID NO: 409 - RNA, 16 nt, synthetic construct
  source 1..16: mol_type = other RNA
tgatttgaagtggaaa

SEQ ID NO: 410 - RNA, 16 nt, synthetic construct
  source 1..16: mol_type = other RNA
gatttgaagtggaaaa

SEQ ID NO: 411 - RNA, 16 nt, synthetic construct
  source 1..16: mol_type = other RNA
cctgaattgtttgata

SEQ ID NO: 412 - RNA, 16 nt, synthetic construct
  source 1..16: mol_type = other RNA
tttgatattgtcacca

SEQ ID NO: 413 - RNA, 16 nt, synthetic construct
  source 1..16: mol_type = other RNA
atattgtcacctagca

SEQ ID NO: 414 - RNA, 16 nt, synthetic construct
  source 1..16: mol_type = other RNA
attgtcacctagcaga

SEQ ID NO: 415 - RNA, 16 nt, synthetic construct
  source 1..16: mol_type = other RNA
tcacctagcagatata

SEQ ID NO: 416 - RNA, 16 nt, synthetic construct
  source 1..16: mol_type = other RNA
cctagcagatatgtaa

SEQ ID NO: 417 - RNA, 16 nt, synthetic construct
  source 1..16: mol_type = other RNA
atatgtattactttta

SEQ ID NO: 418 - RNA, 16 nt, synthetic construct
  source 1..16: mol_type = other RNA
gcaatgttattattga

SEQ ID NO: 419 - RNA, 16 nt, synthetic construct
  source 1..16: mol_type = other RNA
caatgttattattgga

SEQ ID NO: 420 - RNA, 16 nt, synthetic construct
  source 1..16: mol_type = other RNA
ttattattggcttgca

SEQ ID NO: 421 - RNA, 16 nt, synthetic construct
  source 1..16: mol_type = other RNA
ttggcttgcactttga

SEQ ID NO: 422 - RNA, 16 nt, synthetic construct
  source 1..16: mol_type = other RNA
ggcttgcactttgtga

SEQ ID NO: 423 - RNA, 16 nt, synthetic construct
  source 1..16: mol_type = other RNA
cactttgtgagtatta

SEQ ID NO: 424 - RNA, 16 nt, synthetic construct
  source 1..16: mol_type = other RNA
actttgtgagtattca

SEQ ID NO: 425 - RNA, 16 nt, synthetic construct
  source 1..16: mol_type = other RNA
ctttgtgagtattcta

SEQ ID NO: 426 - RNA, 16 nt, synthetic construct
  source 1..16: mol_type = other RNA
tttgtgagtattctaa

SEQ ID NO: 427 - RNA, 16 nt, synthetic construct
  source 1..16: mol_type = other RNA
tttgtgagtattctaa

SEQ ID NO: 428 - RNA, 16 nt, synthetic construct
  source 1..16: mol_type = other RNA
ttgtgagtattctata

SEQ ID NO: 429 - RNA, 16 nt, synthetic construct
  source 1..16: mol_type = other RNA
gtgagtattctatgta

SEQ ID NO: 430 - RNA, 16 nt, synthetic construct
  source 1..16: mol_type = other RNA
ttgcataggacagaca

SEQ ID NO: 431 - RNA, 16 nt, synthetic construct
  source 1..16: mol_type = other RNA
gcataggacagactta

SEQ ID NO: 432 - RNA, 16 nt, synthetic construct
  source 1..16: mol_type = other RNA
cataggacagacttaa

SEQ ID NO: 433 - RNA, 16 nt, synthetic construct
  source 1..16: mol_type = other RNA
ataggacagacttaga

SEQ ID NO: 434 - RNA, 16 nt, synthetic construct
  source 1..16: mol_type = other RNA
aggacagacttaggaa

SEQ ID NO: 435 - RNA, 16 nt, synthetic construct
  source 1..16: mol_type = other RNA
cagacttaggagttta

SEQ ID NO: 436 - RNA, 16 nt, synthetic construct
  source 1..16: mol_type = other RNA
tttgtttagagcagta

SEQ ID NO: 437 - RNA, 16 nt, synthetic construct
  source 1..16: mol_type = other RNA
gagcagttaacatcta

SEQ ID NO: 438 - RNA, 16 nt, synthetic construct
  source 1..16: mol_type = other RNA
agcagttaacatctga

SEQ ID NO: 439 - RNA, 16 nt, synthetic construct
  source 1..16: mol_type = other RNA
gcagttaacatctgaa

SEQ ID NO: 440 - RNA, 16 nt, synthetic construct
  source 1..16: mol_type = other RNA
cagttaacatctgaaa

SEQ ID NO: 441 - RNA, 16 nt, synthetic construct
  source 1..16: mol_type = other RNA
agttaacatctgaaga

SEQ ID NO: 442 - RNA, 16 nt, synthetic construct
  source 1..16: mol_type = other RNA
atctgaagtgtctaaa

SEQ ID NO: 443 - RNA, 16 nt, synthetic construct
  source 1..16: mol_type = other RNA
tgaagtgtctaatgca

SEQ ID NO: 444 - RNA, 16 nt, synthetic construct
  source 1..16: mol_type = other RNA
aatgcattaactttta

SEQ ID NO: 445 - RNA, 16 nt, synthetic construct
  source 1..16: mol_type = other RNA
cttttgtaaggtacta

SEQ ID NO: 446 - RNA, 16 nt, synthetic construct
  source 1..16: mol_type = other RNA
ttttgtaaggtactga

SEQ ID NO: 447 - RNA, 16 nt, synthetic construct
  source 1..16: mol_type = other RNA
tttgtaaggtactgaa

SEQ ID NO: 448 - RNA, 16 nt, synthetic construct
  source 1..16: mol_type = other RNA
ttgtaaggtactgaaa

SEQ ID NO: 449 - RNA, 16 nt, synthetic construct
  source 1..16: mol_type = other RNA
tgtaaggtactgaata

SEQ ID NO: 450 - RNA, 16 nt, synthetic construct
  source 1..16: mol_type = other RNA
ggtactgaatacttaa

SEQ ID NO: 451 - RNA, 16 nt, synthetic construct
  source 1..16: mol_type = other RNA
acttaatatgtgggaa

SEQ ID NO: 452 - RNA, 16 nt, synthetic construct
  source 1..16: mol_type = other RNA
tccttaggcttacaaa

SEQ ID NO: 453 - RNA, 16 nt, synthetic construct
  source 1..16: mol_type = other RNA
ctgaatcgtttcatga

SEQ ID NO: 454 - RNA, 16 nt, synthetic construct
  source 1..16: mol_type = other RNA
gtttcatgtaagaata

SEQ ID NO: 455 - RNA, 16 nt, synthetic construct
  source 1..16: mol_type = other RNA
catgtaagaatccaaa

SEQ ID NO: 456 - RNA, 16 nt, synthetic construct
  source 1..16: mol_type = other RNA
taagaatccaaagtga

SEQ ID NO: 457 - RNA, 16 nt, synthetic construct
  source 1..16: mol_type = other RNA
aaagtggacaccatta

SEQ ID NO: 458 - RNA, 16 nt, synthetic construct
  source 1..16: mol_type = other RNA
agtggacaccattaaa

SEQ ID NO: 459 - RNA, 16 nt, synthetic construct
  source 1..16: mol_type = other RNA
cattaacaggtcttta

SEQ ID NO: 460 - RNA, 16 nt, synthetic construct
  source 1..16: mol_type = other RNA
tctttgaaatatgcaa

SEQ ID NO: 461 - RNA, 16 nt, synthetic construct
  source 1..16: mol_type = other RNA
gtaactttgcatgtta

SEQ ID NO: 462 - RNA, 16 nt, synthetic construct
  source 1..16: mol_type = other RNA
taactttgcatgttca

SEQ ID NO: 463 - RNA, 16 nt, synthetic construct
  source 1..16: mol_type = other RNA
aactttgcatgttcta

SEQ ID NO: 464 - RNA, 16 nt, synthetic construct
  source 1..16: mol_type = other RNA
actttgcatgttctta

SEQ ID NO: 465 - RNA, 16 nt, synthetic construct
  source 1..16: mol_type = other RNA
ctttgcatgttcttga

SEQ ID NO: 466 - RNA, 16 nt, synthetic construct
  source 1..16: mol_type = other RNA
ctttgcatgttcttga

SEQ ID NO: 467 - RNA, 16 nt, synthetic construct
  source 1..16: mol_type = other RNA
tttgcatgttcttgta

SEQ ID NO: 468 - RNA, 16 nt, synthetic construct
  source 1..16: mol_type = other RNA
ttgcatgttcttgtta

SEQ ID NO: 469 - RNA, 16 nt, synthetic construct
  source 1..16: mol_type = other RNA
tgcatgttcttgttta

SEQ ID NO: 470 - RNA, 16 nt, synthetic construct
  source 1..16: mol_type = other RNA
gcatgttcttgtttta

SEQ ID NO: 471 - RNA, 16 nt, synthetic construct
  source 1..16: mol_type = other RNA
catgttcttgttttga

SEQ ID NO: 472 - RNA, 16 nt, synthetic construct
  source 1..16: mol_type = other RNA
catgttcttgttttga

SEQ ID NO: 473 - RNA, 16 nt, synthetic construct
  source 1..16: mol_type = other RNA
atgttcttgttttgta

SEQ ID NO: 474 - RNA, 16 nt, synthetic construct
  source 1..16: mol_type = other RNA
tgactgaaattaaaca

SEQ ID NO: 475 - RNA, 16 nt, synthetic construct
  source 1..16: mol_type = other RNA
tgaaattaaacgagca

SEQ ID NO: 476 - RNA, 16 nt, synthetic construct
  source 1..16: mol_type = other RNA
gaaattaaacgagcga

SEQ ID NO: 477 - RNA, 16 nt, synthetic construct
  source 1..16: mol_type = other RNA
aaattaaacgagcgaa

SEQ ID NO: 478 - RNA, 16 nt, synthetic construct
  source 1..16: mol_type = other RNA
aattaaacgagcgaaa

SEQ ID NO: 479 - RNA, 16 nt, synthetic construct
  source 1..16: mol_type = other RNA
attaaacgagcgaaga

SEQ ID NO: 480 - RNA, 16 nt, synthetic construct
  source 1..16: mol_type = other RNA
gaccgctactaccgta

SEQ ID NO: 481 - RNA, 16 nt, synthetic construct
  source 1..16: mol_type = other RNA
gcacacggtcaccaca

SEQ ID NO: 482 - RNA, 16 nt, synthetic construct
  source 1..16: mol_type = other RNA
atcctcctcatctcca

SEQ ID NO: 483 - RNA, 16 nt, synthetic construct
  source 1..16: mol_type = other RNA
tggtctagtgaataaa

SEQ ID NO: 484 - RNA, 16 nt, synthetic construct
  source 1..16: mol_type = other RNA
ctagtgaataaataca

SEQ ID NO: 485 - RNA, 16 nt, synthetic construct
  source 1..16: mol_type = other RNA
tgggatagctagagca

SEQ ID NO: 486 - RNA, 16 nt, synthetic construct
  source 1..16: mol_type = other RNA
tgacgttgaaagcaaa

SEQ ID NO: 487 - RNA, 16 nt, synthetic construct
  source 1..16: mol_type = other RNA
tcccagggcactagaa

SEQ ID NO: 488 - RNA, 16 nt, synthetic construct
  source 1..16: mol_type = other RNA
caattatcaaaagcta

SEQ ID NO: 489 - RNA, 16 nt, synthetic construct
  source 1..16: mol_type = other RNA
ctatgtttctgtacta

SEQ ID NO: 490 - RNA, 16 nt, synthetic construct
  source 1..16: mol_type = other RNA
gaaagaaattctgcta

SEQ ID NO: 491 - RNA, 16 nt, synthetic construct
  source 1..16: mol_type = other RNA
gcttatgtcttcttta

SEQ ID NO: 492 - RNA, 16 nt, synthetic construct
  source 1..16: mol_type = other RNA
ctttgcatgtatttta

SEQ ID NO: 493 - RNA, 21 nt, Homo sapiens
  source 1..21: mol_type = mRNA
tacagtcaatgagccacgtca

SEQ ID NO: 494 - RNA, 21 nt, Homo sapiens
  source 1..21: mol_type = mRNA
atcaactgatgcaagtgttca

SEQ ID NO: 495 - RNA, 21 nt, Homo sapiens
  source 1..21: mol_type = mRNA
cagcgctgcaccctttaactt

SEQ ID NO: 496 - RNA, 21 nt, Homo sapiens
  source 1..21: mol_type = mRNA
ctgtcctccgagccagtcgct

SEQ ID NO: 497 - RNA, 21 nt, Homo sapiens
  source 1..21: mol_type = mRNA
ccgcgagcttctcctctcctc

SEQ ID NO: 498 - RNA, 21 nt, Homo sapiens
  source 1..21: mol_type = mRNA
gcgagcttctcctctcctcac

SEQ ID NO: 499 - RNA, 21 nt, Homo sapiens
  source 1..21: mol_type = mRNA
gcttctcctctcctcacgacc

SEQ ID NO: 500 - RNA, 21 nt, Homo sapiens
  source 1..21: mol_type = mRNA
aaggaggtggcacccacagtc

SEQ ID NO: 501 - RNA, 21 nt, Homo sapiens
  source 1..21: mol_type = mRNA
ggaacaagccgagtaagccaa

SEQ ID NO: 502 - RNA, 21 nt, Homo sapiens
  source 1..21: mol_type = mRNA
ttggcggctacatgctgggaa

SEQ ID NO: 503 - RNA, 21 nt, Homo sapiens
  source 1..21: mol_type = mRNA
caggcccatcatacatttcgg

SEQ ID NO: 504 - RNA, 21 nt, Homo sapiens
  source 1..21: mol_type = mRNA
tacatttcggcagtgactatg

SEQ ID NO: 505 - RNA, 21 nt, Homo sapiens
  source 1..21: mol_type = mRNA
tttcggcagtgactatgagga

SEQ ID NO: 506 - RNA, 21 nt, Homo sapiens
  source 1..21: mol_type = mRNA
tcggcagtgactatgaggacc

SEQ ID NO: 507 - RNA, 21 nt, Homo sapiens
  source 1..21: mol_type = mRNA
cagtgactatgaggaccgtta

SEQ ID NO: 508 - RNA, 21 nt, Homo sapiens
  source 1..21: mol_type = mRNA
atgaggaccgttactatcgtg

SEQ ID NO: 509 - RNA, 21 nt, Homo sapiens
  source 1..21: mol_type = mRNA
actatcgtgaaaacatgcacc

SEQ ID NO: 510 - RNA, 21 nt, Homo sapiens
  source 1..21: mol_type = mRNA
gaaaacatgcaccgttacccc

SEQ ID NO: 511 - RNA, 21 nt, Homo sapiens
  source 1..21: mol_type = mRNA
ccaaccaagtgtactacaggc

SEQ ID NO: 512 - RNA, 21 nt, Homo sapiens
  source 1..21: mol_type = mRNA
caaccaagtgtactacaggcc

SEQ ID NO: 513 - RNA, 21 nt, Homo sapiens
  source 1..21: mol_type = mRNA
tgtactacaggcccatggatg

SEQ ID NO: 514 - RNA, 21 nt, Homo sapiens
  source 1..21: mol_type = mRNA
actacaggcccatggatgagt

SEQ ID NO: 515 - RNA, 21 nt, Homo sapiens
  source 1..21: mol_type = mRNA
ctacaggcccatggatgagta

SEQ ID NO: 516 - RNA, 21 nt, Homo sapiens
  source 1..21: mol_type = mRNA
ggcccatggatgagtacagca

SEQ ID NO: 517 - RNA, 21 nt, Homo sapiens
  source 1..21: mol_type = mRNA
tggatgagtacagcaaccaga

SEQ ID NO: 518 - RNA, 21 nt, Homo sapiens
  source 1..21: mol_type = mRNA
atgagtacagcaaccagaaca

SEQ ID NO: 519 - RNA, 21 nt, Homo sapiens
  source 1..21: mol_type = mRNA
agtacagcaaccagaacaact

SEQ ID NO: 520 - RNA, 21 nt, Homo sapiens
  source 1..21: mol_type = mRNA
acaactttgtgcacgactgcg

SEQ ID NO: 521 - RNA, 21 nt, Homo sapiens
  source 1..21: mol_type = mRNA
tttgtgcacgactgcgtcaat

SEQ ID NO: 522 - RNA, 21 nt, Homo sapiens
  source 1..21: mol_type = mRNA
ttgtgcacgactgcgtcaata

SEQ ID NO: 523 - RNA, 21 nt, Homo sapiens
  source 1..21: mol_type = mRNA
tgtgcacgactgcgtcaatat

SEQ ID NO: 524 - RNA, 21 nt, Homo sapiens
  source 1..21: mol_type = mRNA
gtgcacgactgcgtcaatatc

SEQ ID NO: 525 - RNA, 21 nt, Homo sapiens
  source 1..21: mol_type = mRNA
tgcacgactgcgtcaatatca

SEQ ID NO: 526 - RNA, 21 nt, Homo sapiens
  source 1..21: mol_type = mRNA
gcacgactgcgtcaatatcac

SEQ ID NO: 527 - RNA, 21 nt, Homo sapiens
  source 1..21: mol_type = mRNA
cgtcaatatcacaatcaagca

SEQ ID NO: 528 - RNA, 21 nt, Homo sapiens
  source 1..21: mol_type = mRNA
tcaatatcacaatcaagcagc

SEQ ID NO: 529 - RNA, 21 nt, Homo sapiens
  source 1..21: mol_type = mRNA
caatatcacaatcaagcagca

SEQ ID NO: 530 - RNA, 21 nt, Homo sapiens
  source 1..21: mol_type = mRNA
atatcacaatcaagcagcaca

SEQ ID NO: 531 - RNA, 21 nt, Homo sapiens
  source 1..21: mol_type = mRNA
atcaagcagcacacggtcacc

SEQ ID NO: 532 - RNA, 21 nt, Homo sapiens
  source 1..21: mol_type = mRNA
tcaagcagcacacggtcacca

SEQ ID NO: 533 - RNA, 21 nt, Homo sapiens
  source 1..21: mol_type = mRNA
aagcagcacacggtcaccaca

SEQ ID NO: 534 - RNA, 21 nt, Homo sapiens
  source 1..21: mol_type = mRNA
cagcacacggtcaccacaacc

SEQ ID NO: 535 - RNA, 21 nt, Homo sapiens
  source 1..21: mol_type = mRNA
ggtcaccacaaccaccaaggg

SEQ ID NO: 536 - RNA, 21 nt, Homo sapiens
  source 1..21: mol_type = mRNA
gggagaacttcaccgagaccg

SEQ ID NO: 537 - RNA, 21 nt, Homo sapiens
  source 1..21: mol_type = mRNA
cgttaagatgatggagcgcgt

SEQ ID NO: 538 - RNA, 21 nt, Homo sapiens
  source 1..21: mol_type = mRNA
taagatgatggagcgcgtggt

SEQ ID NO: 539 - RNA, 21 nt, Homo sapiens
  source 1..21: mol_type = mRNA
tggagcgcgtggttgagcaga

SEQ ID NO: 540 - RNA, 21 nt, Homo sapiens
  source 1..21: mol_type = mRNA
taccagagaggatcgagcatg

SEQ ID NO: 541 - RNA, 21 nt, Homo sapiens
  source 1..21: mol_type = mRNA
atcgagcatggtcctcttctc

SEQ ID NO: 542 - RNA, 21 nt, Homo sapiens
  source 1..21: mol_type = mRNA
cgagcatggtcctcttctcct

SEQ ID NO: 543 - RNA, 21 nt, Homo sapiens
  source 1..21: mol_type = mRNA
ctctccacctgtgatcctcct

SEQ ID NO: 544 - RNA, 21 nt, Homo sapiens
  source 1..21: mol_type = mRNA
ctgtgatcctcctgatctctt

SEQ ID NO: 545 - RNA, 21 nt, Homo sapiens
  source 1..21: mol_type = mRNA
gatcctcctgatctctttcct

SEQ ID NO: 546 - RNA, 21 nt, Homo sapiens
  source 1..21: mol_type = mRNA
ctctttcctcatcttcctgat

SEQ ID NO: 547 - RNA, 21 nt, Homo sapiens
  source 1..21: mol_type = mRNA
gtgggatgaggaaggtcttcc

SEQ ID NO: 548 - RNA, 21 nt, Homo sapiens
  source 1..21: mol_type = mRNA
ttcctgttttcaccatctttc

SEQ ID NO: 549 - RNA, 21 nt, Homo sapiens
  source 1..21: mol_type = mRNA
tcctgttttcaccatctttct

SEQ ID NO: 550 - RNA, 21 nt, Homo sapiens
  source 1..21: mol_type = mRNA
ctgttttcaccatctttctaa

SEQ ID NO: 551 - RNA, 21 nt, Homo sapiens
  source 1..21: mol_type = mRNA
tttcaccatctttctaatctt

SEQ ID NO: 552 - RNA, 21 nt, Homo sapiens
  source 1..21: mol_type = mRNA
ctctctttgtcccggataggc

SEQ ID NO: 553 - RNA, 21 nt, Homo sapiens
  source 1..21: mol_type = mRNA
tttgtcccggataggctaatc

SEQ ID NO: 554 - RNA, 21 nt, Homo sapiens
  source 1..21: mol_type = mRNA
tgtcccggataggctaatcaa

SEQ ID NO: 555 - RNA, 21 nt, Homo sapiens
  source 1..21: mol_type = mRNA
gtcccggataggctaatcaat

SEQ ID NO: 556 - RNA, 21 nt, Homo sapiens
  source 1..21: mol_type = mRNA
gatgggcactggaaaacatag

SEQ ID NO: 557 - RNA, 21 nt, Homo sapiens
  source 1..21: mol_type = mRNA
tgctaatgccaggccagtaaa

SEQ ID NO: 558 - RNA, 21 nt, Homo sapiens
  source 1..21: mol_type = mRNA
taatgccaggccagtaaaagt

SEQ ID NO: 559 - RNA, 21 nt, Homo sapiens
  source 1..21: mol_type = mRNA
aacagcaaataaccattggtt

SEQ ID NO: 560 - RNA, 21 nt, Homo sapiens
  source 1..21: mol_type = mRNA
attggttaatctggacttatt

SEQ ID NO: 561 - RNA, 21 nt, Homo sapiens
  source 1..21: mol_type = mRNA
aacaggttgaggctaaaacaa

SEQ ID NO: 562 - RNA, 21 nt, Homo sapiens
  source 1..21: mol_type = mRNA
gttgaggctaaaacaaatctc

SEQ ID NO: 563 - RNA, 21 nt, Homo sapiens
  source 1..21: mol_type = mRNA
ttgaggctaaaacaaatctca

SEQ ID NO: 564 - RNA, 21 nt, Homo sapiens
  source 1..21: mol_type = mRNA
tgaggctaaaacaaatctcag

SEQ ID NO: 565 - RNA, 21 nt, Homo sapiens
  source 1..21: mol_type = mRNA
gaggctaaaacaaatctcaga

SEQ ID NO: 566 - RNA, 21 nt, Homo sapiens
  source 1..21: mol_type = mRNA
aggctaaaacaaatctcagaa

SEQ ID NO: 567 - RNA, 21 nt, Homo sapiens
  source 1..21: mol_type = mRNA
caaatctcagaacagtctgaa

SEQ ID NO: 568 - RNA, 21 nt, Homo sapiens
  source 1..21: mol_type = mRNA
agaacagtctgaaataccttt

SEQ ID NO: 569 - RNA, 21 nt, Homo sapiens
  source 1..21: mol_type = mRNA
ggatacctctggctccttcag

SEQ ID NO: 570 - RNA, 21 nt, Homo sapiens
  source 1..21: mol_type = mRNA
gatacctctggctccttcagc

SEQ ID NO: 571 - RNA, 21 nt, Homo sapiens
  source 1..21: mol_type = mRNA
atacctctggctccttcagca

SEQ ID NO: 572 - RNA, 21 nt, Homo sapiens
  source 1..21: mol_type = mRNA
agtatactaatgccctatctt

SEQ ID NO: 573 - RNA, 21 nt, Homo sapiens
  source 1..21: mol_type = mRNA
gtatactaatgccctatctta

SEQ ID NO: 574 - RNA, 21 nt, Homo sapiens
  source 1..21: mol_type = mRNA
ccctatcttagtagagatttc

SEQ ID NO: 575 - RNA, 21 nt, Homo sapiens
  source 1..21: mol_type = mRNA
cctatcttagtagagatttca

SEQ ID NO: 576 - RNA, 21 nt, Homo sapiens
  source 1..21: mol_type = mRNA
agtagagatttcatagctatt

SEQ ID NO: 577 - RNA, 21 nt, Homo sapiens
  source 1..21: mol_type = mRNA
agagatttcatagctatttag

SEQ ID NO: 578 - RNA, 21 nt, Homo sapiens
  source 1..21: mol_type = mRNA
tccattttaagaaaacccgac

SEQ ID NO: 579 - RNA, 21 nt, Homo sapiens
  source 1..21: mol_type = mRNA
ccattttaagaaaacccgaca

SEQ ID NO: 580 - RNA, 21 nt, Homo sapiens
  source 1..21: mol_type = mRNA
ccaggtttgttaggaggccac

SEQ ID NO: 581 - RNA, 21 nt, Homo sapiens
  source 1..21: mol_type = mRNA
tgttaggaggccacatgatac

SEQ ID NO: 582 - RNA, 21 nt, Homo sapiens
  source 1..21: mol_type = mRNA
gttaggaggccacatgatact

SEQ ID NO: 583 - RNA, 21 nt, Homo sapiens
  source 1..21: mol_type = mRNA
aggaggccacatgatacttat

SEQ ID NO: 584 - RNA, 21 nt, Homo sapiens
  source 1..21: mol_type = mRNA
tagagattcttagctcttggg

SEQ ID NO: 585 - RNA, 21 nt, Homo sapiens
  source 1..21: mol_type = mRNA
gctctgtgtgtaccgagaact

SEQ ID NO: 586 - RNA, 21 nt, Homo sapiens
  source 1..21: mol_type = mRNA
tgttttacttttcacagtatg

SEQ ID NO: 587 - RNA, 21 nt, Homo sapiens
  source 1..21: mol_type = mRNA
tcaacaagagtaaatattgtc

SEQ ID NO: 588 - RNA, 21 nt, Homo sapiens
  source 1..21: mol_type = mRNA
ctctggctagaggacatattc

SEQ ID NO: 589 - RNA, 21 nt, Homo sapiens
  source 1..21: mol_type = mRNA
tctggctagaggacatattca

SEQ ID NO: 590 - RNA, 21 nt, Homo sapiens
  source 1..21: mol_type = mRNA
ttcacagtgaacataactgta

SEQ ID NO: 591 - RNA, 21 nt, Homo sapiens
  source 1..21: mol_type = mRNA
tcacagtgaacataactgtaa

SEQ ID NO: 592 - RNA, 21 nt, Homo sapiens
  source 1..21: mol_type = mRNA
aacatatatgaaaggcttctg

SEQ ID NO: 593 - RNA, 21 nt, Homo sapiens
  source 1..21: mol_type = mRNA
gaaaggcttctgggacttgaa

SEQ ID NO: 594 - RNA, 21 nt, Homo sapiens
  source 1..21: mol_type = mRNA
tctgggacttgaaatcaaatg

SEQ ID NO: 595 - RNA, 21 nt, Homo sapiens
  source 1..21: mol_type = mRNA
acctcccattttagatgttta

SEQ ID NO: 596 - RNA, 21 nt, Homo sapiens
  source 1..21: mol_type = mRNA
atgtttaaaggaccctatatg

SEQ ID NO: 597 - RNA, 21 nt, Homo sapiens
  source 1..21: mol_type = mRNA
atatgtggcattcctttcttt

SEQ ID NO: 598 - RNA, 21 nt, Homo sapiens
  source 1..21: mol_type = mRNA
tcctttctttaaactataggt

SEQ ID NO: 599 - RNA, 21 nt, Homo sapiens
  source 1..21: mol_type = mRNA
tataggtaattaaggcagctg

SEQ ID NO: 600 - RNA, 21 nt, Homo sapiens
  source 1..21: mol_type = mRNA
ataggtaattaaggcagctga

SEQ ID NO: 601 - RNA, 21 nt, Homo sapiens
  source 1..21: mol_type = mRNA
aggtaattaaggcagctgaaa

SEQ ID NO: 602 - RNA, 21 nt, Homo sapiens
  source 1..21: mol_type = mRNA
aattaaggcagctgaaaagta

SEQ ID NO: 603 - RNA, 21 nt, Homo sapiens
  source 1..21: mol_type = mRNA
attaaggcagctgaaaagtaa

SEQ ID NO: 604 - RNA, 21 nt, Homo sapiens
  source 1..21: mol_type = mRNA
ttaaggcagctgaaaagtaaa

SEQ ID NO: 605 - RNA, 21 nt, Homo sapiens
  source 1..21: mol_type = mRNA
taaggcagctgaaaagtaaat

SEQ ID NO: 606 - RNA, 21 nt, Homo sapiens
  source 1..21: mol_type = mRNA
aaggcagctgaaaagtaaatt

SEQ ID NO: 607 - RNA, 21 nt, Homo sapiens
  source 1..21: mol_type = mRNA
cagctgaaaagtaaattgcct

SEQ ID NO: 608 - RNA, 21 nt, Homo sapiens
  source 1..21: mol_type = mRNA
aaaagtaaattgccttctaga

SEQ ID NO: 609 - RNA, 21 nt, Homo sapiens
  source 1..21: mol_type = mRNA
aattgccttctagacactgaa

SEQ ID NO: 610 - RNA, 21 nt, Homo sapiens
  source 1..21: mol_type = mRNA
cttctagacactgaaggcaaa

SEQ ID NO: 611 - RNA, 21 nt, Homo sapiens
  source 1..21: mol_type = mRNA
tgaaggcaaatctcctttgtc

SEQ ID NO: 612 - RNA, 21 nt, Homo sapiens
  source 1..21: mol_type = mRNA
gcaaatctcctttgtccattt

SEQ ID NO: 613 - RNA, 21 nt, Homo sapiens
  source 1..21: mol_type = mRNA
tttgtccatttacctggaaac

SEQ ID NO: 614 - RNA, 21 nt, Homo sapiens
  source 1..21: mol_type = mRNA
tgtccatttacctggaaacca

SEQ ID NO: 615 - RNA, 21 nt, Homo sapiens
  source 1..21: mol_type = mRNA
ccatttacctggaaaccagaa

SEQ ID NO: 616 - RNA, 21 nt, Homo sapiens
  source 1..21: mol_type = mRNA
tggaaaccagaatgattttga

SEQ ID NO: 617 - RNA, 21 nt, Homo sapiens
  source 1..21: mol_type = mRNA
ttttgacatacaggagagctg

SEQ ID NO: 618 - RNA, 21 nt, Homo sapiens
  source 1..21: mol_type = mRNA
agagctgcagttgtgaaagca

SEQ ID NO: 619 - RNA, 21 nt, Homo sapiens
  source 1..21: mol_type = mRNA
gcagttgtgaaagcaccatca

SEQ ID NO: 620 - RNA, 21 nt, Homo sapiens
  source 1..21: mol_type = mRNA
aagcaccatcatcatagagga

SEQ ID NO: 621 - RNA, 21 nt, Homo sapiens
  source 1..21: mol_type = mRNA
caccatcatcatagaggatga

SEQ ID NO: 622 - RNA, 21 nt, Homo sapiens
  source 1..21: mol_type = mRNA
tcatcatagaggatgatgtaa

SEQ ID NO: 623 - RNA, 21 nt, Homo sapiens
  source 1..21: mol_type = mRNA
aatggtcagtgtgcaaagaaa

SEQ ID NO: 624 - RNA, 21 nt, Homo sapiens
  source 1..21: mol_type = mRNA
ggtcagtgtgcaaagaaaaga

SEQ ID NO: 625 - RNA, 21 nt, Homo sapiens
  source 1..21: mol_type = mRNA
tcagtgtgcaaagaaaagaac

SEQ ID NO: 626 - RNA, 21 nt, Homo sapiens
  source 1..21: mol_type = mRNA
gtgtgcaaagaaaagaactgc

SEQ ID NO: 627 - RNA, 21 nt, Homo sapiens
  source 1..21: mol_type = mRNA
tgtgcaaagaaaagaactgct

SEQ ID NO: 628 - RNA, 21 nt, Homo sapiens
  source 1..21: mol_type = mRNA
gtgcaaagaaaagaactgctt

SEQ ID NO: 629 - RNA, 21 nt, Homo sapiens
  source 1..21: mol_type = mRNA
ctgcttgcatttctttatttc

SEQ ID NO: 630 - RNA, 21 nt, Homo sapiens
  source 1..21: mol_type = mRNA
tgcttgcatttctttatttct

SEQ ID NO: 631 - RNA, 21 nt, Homo sapiens
  source 1..21: mol_type = mRNA
gcttgcatttctttatttctg

SEQ ID NO: 632 - RNA, 21 nt, Homo sapiens
  source 1..21: mol_type = mRNA
gcttgcatttctttatttctg

SEQ ID NO: 633 - RNA, 21 nt, Homo sapiens
  source 1..21: mol_type = mRNA
tatttctgtctcataattgtc

SEQ ID NO: 634 - RNA, 21 nt, Homo sapiens
  source 1..21: mol_type = mRNA
ttctgtctcataattgtcaaa

SEQ ID NO: 635 - RNA, 21 nt, Homo sapiens
  source 1..21: mol_type = mRNA
aattaggtcaagttcatagtt

SEQ ID NO: 636 - RNA, 21 nt, Homo sapiens
  source 1..21: mol_type = mRNA
attaggtcaagttcatagttt

SEQ ID NO: 637 - RNA, 21 nt, Homo sapiens
  source 1..21: mol_type = mRNA
ttaggtcaagttcatagtttc

SEQ ID NO: 638 - RNA, 21 nt, Homo sapiens
  source 1..21: mol_type = mRNA
taggtcaagttcatagtttct

SEQ ID NO: 639 - RNA, 21 nt, Homo sapiens
  source 1..21: mol_type = mRNA
aggtcaagttcatagtttctg

SEQ ID NO: 640 - RNA, 21 nt, Homo sapiens
  source 1..21: mol_type = mRNA
ggtcaagttcatagtttctgt

SEQ ID NO: 641 - RNA, 21 nt, Homo sapiens
  source 1..21: mol_type = mRNA
gttcatagtttctgtaattgg

SEQ ID NO: 642 - RNA, 21 nt, Homo sapiens
  source 1..21: mol_type = mRNA
tagtttctgtaattggctttt

SEQ ID NO: 643 - RNA, 21 nt, Homo sapiens
  source 1..21: mol_type = mRNA
tttctgtaattggcttttgaa

SEQ ID NO: 644 - RNA, 21 nt, Homo sapiens
  source 1..21: mol_type = mRNA
ctgtaattggcttttgaatca

SEQ ID NO: 645 - RNA, 21 nt, Homo sapiens
  source 1..21: mol_type = mRNA
tgtaattggcttttgaatcaa

SEQ ID NO: 646 - RNA, 21 nt, Homo sapiens
  source 1..21: mol_type = mRNA
ttggcttttgaatcaaagaat

SEQ ID NO: 647 - RNA, 21 nt, Homo sapiens
  source 1..21: mol_type = mRNA
gcttttgaatcaaagaatagg

SEQ ID NO: 648 - RNA, 21 nt, Homo sapiens
  source 1..21: mol_type = mRNA
cttttgaatcaaagaataggg

SEQ ID NO: 649 - RNA, 21 nt, Homo sapiens
  source 1..21: mol_type = mRNA
ttgaatcaaagaatagggaga

SEQ ID NO: 650 - RNA, 21 nt, Homo sapiens
  source 1..21: mol_type = mRNA
aagaatagggagacaatctaa

SEQ ID NO: 651 - RNA, 21 nt, Homo sapiens
  source 1..21: mol_type = mRNA
cttaggttggagatgacagaa

SEQ ID NO: 652 - RNA, 21 nt, Homo sapiens
  source 1..21: mol_type = mRNA
ttaggttggagatgacagaaa

SEQ ID NO: 653 - RNA, 21 nt, Homo sapiens
  source 1..21: mol_type = mRNA
taggttggagatgacagaaat

SEQ ID NO: 654 - RNA, 21 nt, Homo sapiens
  source 1..21: mol_type = mRNA
gttggagatgacagaaatatg

SEQ ID NO: 655 - RNA, 21 nt, Homo sapiens
  source 1..21: mol_type = mRNA
atgattgatttgaagtggaaa

SEQ ID NO: 656 - RNA, 21 nt, Homo sapiens
  source 1..21: mol_type = mRNA
tgattgatttgaagtggaaaa

SEQ ID NO: 657 - RNA, 21 nt, Homo sapiens
  source 1..21: mol_type = mRNA
tattccctgaattgtttgata

SEQ ID NO: 658 - RNA, 21 nt, Homo sapiens
  source 1..21: mol_type = mRNA
aattgtttgatattgtcacct

SEQ ID NO: 659 - RNA, 21 nt, Homo sapiens
  source 1..21: mol_type = mRNA
gtttgatattgtcacctagca

SEQ ID NO: 660 - RNA, 21 nt, Homo sapiens
  source 1..21: mol_type = mRNA
ttgatattgtcacctagcaga

SEQ ID NO: 661 - RNA, 21 nt, Homo sapiens
  source 1..21: mol_type = mRNA
tattgtcacctagcagatatg

SEQ ID NO: 662 - RNA, 21 nt, Homo sapiens
  source 1..21: mol_type = mRNA
tgtcacctagcagatatgtat

SEQ ID NO: 663 - RNA, 21 nt, Homo sapiens
  source 1..21: mol_type = mRNA
agcagatatgtattacttttc

SEQ ID NO: 664 - RNA, 21 nt, Homo sapiens
  source 1..21: mol_type = mRNA
tttctgcaatgttattattgg

SEQ ID NO: 665 - RNA, 21 nt, Homo sapiens
  source 1..21: mol_type = mRNA
ttctgcaatgttattattggc

SEQ ID NO: 666 - RNA, 21 nt, Homo sapiens
  source 1..21: mol_type = mRNA
caatgttattattggcttgca

SEQ ID NO: 667 - RNA, 21 nt, Homo sapiens
  source 1..21: mol_type = mRNA
tattattggcttgcactttgt

SEQ ID NO: 668 - RNA, 21 nt, Homo sapiens
  source 1..21: mol_type = mRNA
ttattggcttgcactttgtga

SEQ ID NO: 669 - RNA, 21 nt, Homo sapiens
  source 1..21: mol_type = mRNA
gcttgcactttgtgagtattc

SEQ ID NO: 670 - RNA, 21 nt, Homo sapiens
  source 1..21: mol_type = mRNA
cttgcactttgtgagtattct

SEQ ID NO: 671 - RNA, 21 nt, Homo sapiens
  source 1..21: mol_type = mRNA
ttgcactttgtgagtattcta

SEQ ID NO: 672 - RNA, 21 nt, Homo sapiens
  source 1..21: mol_type = mRNA
tgcactttgtgagtattctat

SEQ ID NO: 673 - RNA, 21 nt, Homo sapiens
  source 1..21: mol_type = mRNA
tgcactttgtgagtattctat

SEQ ID NO: 674 - RNA, 21 nt, Homo sapiens
  source 1..21: mol_type = mRNA
gcactttgtgagtattctatg

SEQ ID NO: 675 - RNA, 21 nt, Homo sapiens
  source 1..21: mol_type = mRNA
actttgtgagtattctatgta

SEQ ID NO: 676 - RNA, 21 nt, Homo sapiens
  source 1..21: mol_type = mRNA
atatattgcataggacagact

SEQ ID NO: 677 - RNA, 21 nt, Homo sapiens
  source 1..21: mol_type = mRNA
atattgcataggacagactta

SEQ ID NO: 678 - RNA, 21 nt, Homo sapiens
  source 1..21: mol_type = mRNA
tattgcataggacagacttag

SEQ ID NO: 679 - RNA, 21 nt, Homo sapiens
  source 1..21: mol_type = mRNA
attgcataggacagacttagg

SEQ ID NO: 680 - RNA, 21 nt, Homo sapiens
  source 1..21: mol_type = mRNA
tgcataggacagacttaggag

SEQ ID NO: 681 - RNA, 21 nt, Homo sapiens
  source 1..21: mol_type = mRNA
taggacagacttaggagtttt

SEQ ID NO: 682 - RNA, 21 nt, Homo sapiens
  source 1..21: mol_type = mRNA
ggagttttgtttagagcagtt

SEQ ID NO: 683 - RNA, 21 nt, Homo sapiens
  source 1..21: mol_type = mRNA
gtttagagcagttaacatctg

SEQ ID NO: 684 - RNA, 21 nt, Homo sapiens
  source 1..21: mol_type = mRNA
tttagagcagttaacatctga

SEQ ID NO: 685 - RNA, 21 nt, Homo sapiens
  source 1..21: mol_type = mRNA
ttagagcagttaacatctgaa

SEQ ID NO: 686 - RNA, 21 nt, Homo sapiens
  source 1..21: mol_type = mRNA
tagagcagttaacatctgaag

SEQ ID NO: 687 - RNA, 21 nt, Homo sapiens
  source 1..21: mol_type = mRNA
agagcagttaacatctgaagt

SEQ ID NO: 688 - RNA, 21 nt, Homo sapiens
  source 1..21: mol_type = mRNA
ttaacatctgaagtgtctaat

SEQ ID NO: 689 - RNA, 21 nt, Homo sapiens
  source 1..21: mol_type = mRNA
acatctgaagtgtctaatgca

SEQ ID NO: 690 - RNA, 21 nt, Homo sapiens
  source 1..21: mol_type = mRNA
tgtctaatgcattaacttttg

SEQ ID NO: 691 - RNA, 21 nt, Homo sapiens
  source 1..21: mol_type = mRNA
attaacttttgtaaggtactg

SEQ ID NO: 692 - RNA, 21 nt, Homo sapiens
  source 1..21: mol_type = mRNA
ttaacttttgtaaggtactga

SEQ ID NO: 693 - RNA, 21 nt, Homo sapiens
  source 1..21: mol_type = mRNA
taacttttgtaaggtactgaa

SEQ ID NO: 694 - RNA, 21 nt, Homo sapiens
  source 1..21: mol_type = mRNA
aacttttgtaaggtactgaat

SEQ ID NO: 695 - RNA, 21 nt, Homo sapiens
  source 1..21: mol_type = mRNA
acttttgtaaggtactgaata

SEQ ID NO: 696 - RNA, 21 nt, Homo sapiens
  source 1..21: mol_type = mRNA
tgtaaggtactgaatacttaa

SEQ ID NO: 697 - RNA, 21 nt, Homo sapiens
  source 1..21: mol_type = mRNA
tgaatacttaatatgtgggaa

SEQ ID NO: 698 - RNA, 21 nt, Homo sapiens
  source 1..21: mol_type = mRNA
cgtggtccttaggcttacaat

SEQ ID NO: 699 - RNA, 21 nt, Homo sapiens
  source 1..21: mol_type = mRNA
gtgcactgaatcgtttcatgt

SEQ ID NO: 700 - RNA, 21 nt, Homo sapiens
  source 1..21: mol_type = mRNA
gaatcgtttcatgtaagaatc

SEQ ID NO: 701 - RNA, 21 nt, Homo sapiens
  source 1..21: mol_type = mRNA
cgtttcatgtaagaatccaaa

SEQ ID NO: 702 - RNA, 21 nt, Homo sapiens
  source 1..21: mol_type = mRNA
tcatgtaagaatccaaagtgg

SEQ ID NO: 703 - RNA, 21 nt, Homo sapiens
  source 1..21: mol_type = mRNA
aatccaaagtggacaccatta

SEQ ID NO: 704 - RNA, 21 nt, Homo sapiens
  source 1..21: mol_type = mRNA
tccaaagtggacaccattaac

SEQ ID NO: 705 - RNA, 21 nt, Homo sapiens
  source 1..21: mol_type = mRNA
gacaccattaacaggtctttg

SEQ ID NO: 706 - RNA, 21 nt, Homo sapiens
  source 1..21: mol_type = mRNA
acaggtctttgaaatatgcat

SEQ ID NO: 707 - RNA, 21 nt, Homo sapiens
  source 1..21: mol_type = mRNA
tatttgtaactttgcatgttc

SEQ ID NO: 708 - RNA, 21 nt, Homo sapiens
  source 1..21: mol_type = mRNA
atttgtaactttgcatgttct

SEQ ID NO: 709 - RNA, 21 nt, Homo sapiens
  source 1..21: mol_type = mRNA
tttgtaactttgcatgttctt

SEQ ID NO: 710 - RNA, 21 nt, Homo sapiens
  source 1..21: mol_type = mRNA
ttgtaactttgcatgttcttg

SEQ ID NO: 711 - RNA, 21 nt, Homo sapiens
  source 1..21: mol_type = mRNA
tgtaactttgcatgttcttgt

SEQ ID NO: 712 - RNA, 21 nt, Homo sapiens
  source 1..21: mol_type = mRNA
tgtaactttgcatgttcttgt

SEQ ID NO: 713 - RNA, 21 nt, Homo sapiens
  source 1..21: mol_type = mRNA
gtaactttgcatgttcttgtt

SEQ ID NO: 714 - RNA, 21 nt, Homo sapiens
  source 1..21: mol_type = mRNA
taactttgcatgttcttgttt

SEQ ID NO: 715 - RNA, 21 nt, Homo sapiens
  source 1..21: mol_type = mRNA
aactttgcatgttcttgtttt

SEQ ID NO: 716 - RNA, 21 nt, Homo sapiens
  source 1..21: mol_type = mRNA
actttgcatgttcttgttttg

SEQ ID NO: 717 - RNA, 21 nt, Homo sapiens
  source 1..21: mol_type = mRNA
ctttgcatgttcttgttttgt

SEQ ID NO: 718 - RNA, 21 nt, Homo sapiens
  source 1..21: mol_type = mRNA
ctttgcatgttcttgttttgt

SEQ ID NO: 719 - RNA, 21 nt, Homo sapiens
  source 1..21: mol_type = mRNA
tttgcatgttcttgttttgtt

SEQ ID NO: 720 - RNA, 21 nt, Homo sapiens
  source 1..21: mol_type = mRNA
atatctgactgaaattaaacg

SEQ ID NO: 721 - RNA, 21 nt, Homo sapiens
  source 1..21: mol_type = mRNA
ctgactgaaattaaacgagcg

SEQ ID NO: 722 - RNA, 21 nt, Homo sapiens
  source 1..21: mol_type = mRNA
tgactgaaattaaacgagcga

SEQ ID NO: 723 - RNA, 21 nt, Homo sapiens
  source 1..21: mol_type = mRNA
gactgaaattaaacgagcgaa

SEQ ID NO: 724 - RNA, 21 nt, Homo sapiens
  source 1..21: mol_type = mRNA
actgaaattaaacgagcgaag

SEQ ID NO: 725 - RNA, 21 nt, Homo sapiens
  source 1..21: mol_type = mRNA
ctgaaattaaacgagcgaaga

SEQ ID NO: 726 - RNA, 21 nt, Mus musculus
  source 1..21: mol_type = mRNA
gggaggaccgctactaccgtg

SEQ ID NO: 727 - RNA, 21 nt, Mus musculus
  source 1..21: mol_type = mRNA
aagcagcacacggtcaccacc

SEQ ID NO: 728 - RNA, 21 nt, Mus musculus
  source 1..21: mol_type = mRNA
ctgtcatcctcctcatctcct

SEQ ID NO: 729 - RNA, 21 nt, Mus musculus
  source 1..21: mol_type = mRNA
tgttttggtctagtgaataaa

SEQ ID NO: 730 - RNA, 21 nt, Mus musculus
  source 1..21: mol_type = mRNA
ttggtctagtgaataaatact

SEQ ID NO: 731 - RNA, 21 nt, Mus musculus
  source 1..21: mol_type = mRNA
ccacctgggatagctagagca

SEQ ID NO: 732 - RNA, 21 nt, Mus musculus
  source 1..21: mol_type = mRNA
cctactgacgttgaaagcaaa

SEQ ID NO: 733 - RNA, 21 nt, Mus musculus
  source 1..21: mol_type = mRNA
ttcattcccagggcactagaa

SEQ ID NO: 734 - RNA, 21 nt, Mus musculus
  source 1..21: mol_type = mRNA
tctcacaattatcaaaagcta

SEQ ID NO: 735 - RNA, 21 nt, Mus musculus
  source 1..21: mol_type = mRNA
ctgccctatgtttctgtactt

SEQ ID NO: 736 - RNA, 21 nt, Mus musculus
  source 1..21: mol_type = mRNA
agtgggaaagaaattctgcta

SEQ ID NO: 737 - RNA, 21 nt, Mus musculus
  source 1..21: mol_type = mRNA
ttcaagcttatgtcttctttt

SEQ ID NO: 738 - RNA, 21 nt, Mus musculus
  source 1..21: mol_type = mRNA
tgtaactttgcatgtattttg